(19) United States
(12) Patent Application Publication
(10) Pub. No.: US 2019/0216891 A1
(43) Pub. Date: Jul. 18, 2019

(54) METHOD FOR MODULATING MYELINATION

(71) Applicant: Yeda Research and Development Co., Ltd., Rehovot (IL)

(72) Inventors: Elior PELES, Rehovot (IL); Yang HYUNJEONG, Rehovot (IL)

(21) Appl. No.: 16/081,990

(22) PCT Filed: Mar. 6, 2017
(86) PCT No.: PCT/IL2017/050276

§ 371 Date: Sep. 4, 2018

Related U.S. Application Data
(60) Provisional application No. 62/304,242, filed on Mar. 6, 2016.

(51) Int. Cl. A61K 38/17; A61K 35/15; G01N 33/50; A61P 25/00

(52) U.S. Cl. CPC A61K 38/177; A61P 25/00; G01N 33/5058; A61K 35/15

(57) ABSTRACT

A method of enhancing the myelinating activity of oligodendrocytes or progenitors thereof is disclosed. The method comprises contacting the oligodendrocytes or the progenitors with an agent that binds to G Protein-Coupled Receptor 37 (GPR37) or a polynucleotide encoding same or an upstream activator of the GPR37 so as to up-regulate an amount and/or activity of Extracellular Signal-Regulated Kinase 1/2 (ERK1/2) in the oligodendrocytes or the progenitors.

### FIELD AND BACKGROUND OF THE INVENTION

The present invention, in some embodiments thereof, relates to methods of modulating neuronal myelination using agents that target G Protein-Coupled Receptor 37 (GPR37) and, more particularly, but not exclusively, to use of the agents for treating diseases associated with aberrant myelination.

The myelin membrane, produced by Schwann cells in the peripheral nervous system (PNS) and oligodendrocytes in the central nervous system (CNS) enables energy-efficient saltatory conduction, and provides essential trophic support to maintain axonal integrity and survival. Myelination is a late developmental process that continues to be remodeled throughout life, suggesting that it contributes to nervous system plasticity. Destruction of myelin in the CNS not only leads to devastating white matter diseases such as leukodystrophies and multiple sclerosis, but is also associated with psychiatric disorders, and neurodegenerative diseases. Hence, understanding the mechanisms underlying oligodendrocyte development and myelination, as well as their maintenance and ability to remyelinate after damage, is of great clinical interest.

During development, oligodendrocyte precursor cells (OPCs) differentiate into post-mitotic premyelinating oligodendrocytes, which later on continue to myelinate. Several signaling pathways control the intricate balance between OPC proliferation and differentiation. Myelination in the CNS is regulated by both inhibitory (e.g. PSA-NCAM, WNT, LINGO, GPR17, and Notch-1), and stimulatory (e.g.laminin-α2¹⁴, BDNF and FGF receptor 2) signals. Nevertheless, it is not clear whether once OPCs exit the cell cycle and begin to differentiate, additional extrinsic signals are required for the progression from pre-myelinating to myelinating oligodendrocytes.

Members of the G-protein coupled receptors (GPCRs) superfamily are emerging as important regulators of myelination. For example, in the PNS, the initiation of myelination requires the presence of the adhesion-type GPR126 in Schwann cells. PNS myelin formation and maintenance are also modulated by GPR44, which is activated by prostaglandin D2. In the CNS, OPCs proliferation and early differentiation are regulated by the adhesion-type GPR56 protein and GPR17 respectively. Mutations in GPR56 cause bilateral frontoparietal polymicrogyria (BFPP) disease, which is also characterized by white matter reduction. In addition, several other GPCRs were implicated in remyelination but their roles in myelination during development remains to be determined.

G protein-coupled receptor 37 (Gpr37) shares significant homology with the receptors of endothelin and bombesin peptides. As revealed by cell-specific gene expression analysis, GPR37 is predominantly found in pre-myelinating and myelinating oligodendrocytes, but not in OPCs. It is also present in certain neuronal subsets, such as dopaminergic neurons in the substantia nigra. GPR37 is also known as parkin-associated endothelin B-like receptor (PAEL-R), which was identified as one of the substrates of the E3 ubiquitin ligase parkin.

Background art includes Golan, N. et al. Glia 56, 1176-86 (2008); Brockschnieder, D., et al., J Neurosci 26, 757-62 (2006) and Zhang, Y. et al. J Neurosci 34, 11929-47 (2014).

Additional background art includes US Patent Application No. 20060240523.

### SUMMARY OF THE INVENTION

According to an aspect of some embodiments of the present invention there is provided a method of enhancing the myelinating activity of oligodendrocytes or progenitors thereof comprising contacting the oligodendrocytes or the progenitors with an agent that binds to G Protein-Coupled Receptor 37 (GPR37) or a polynucleotide encoding same or an upstream activator of the GPR37 so as to up-regulate an amount and/or activity of Extracellular Signal-Regulated Kinase 1/2 (ERK1/2) in the oligodendrocytes or the progenitors, thereby enhancing the myelinating activity of the oligodendrocytes or the progenitors.

According to an aspect of some embodiments of the present invention there is provided a method of treating a demyelinating disease of a subject in need thereof, comprising:

(a) contacting oligodendrocytes or progenitors thereof with an agent that binds to GPR37 or a polynucleotide encoding same or an upstream activator of the GPR37 so as to generate cells with an enhanced myelinating activity; and

(b) providing the cells with an enhanced myelinating activity to the subject, thereby treating the demyelinating disease.

According to an aspect of some embodiments of the present invention there is provided a method of treating a demyelinating disease of a subject in need thereof, comprising administering to the subject a therapeutically effective amount of an agent that binds to GPR37 or a polynucleotide encoding same or an upstream activator of the GPR37 so as to up-regulate an amount and/or activity of ERK1/2 in oligodendrocytes or progenitors of the subject, thereby treating the demyelinating disease.

According to an aspect of some embodiments of the present invention there is provided a method of selecting an agent which is capable of increasing the myelinating activity of an oligodendrocyte comprising:

(a) contacting cells which express GPR37 with a candidate agent; and

(b) measuring an amount and/or activity of the GPR37, wherein when the amount and/or activity is below a predetermined level, it is indicative that the candidate agent is capable of increasing the myelinating activity of an oligodendrocyte.

According to some embodiments of the invention, the demyelinating disease is selected from the group consisting of multiple sclerosis, optic neuritis, an idiopathic inflammatory demyelinating disease, Guillain-Barre Syndrome, chronic inflammatory demyelinating polyneuropathy, transverse myelitis, Balo concentric sclerosis, pernicious anemia, central pontine myelinolysis, Tabes dorsalis, neuromyelitis optica (NMO), progressive multifocal leukoencephalopathy (PML), anti-MAG (myelin-associated glycoprotein) neuropathy, hereditary motor and sensory neuropathy (Chacot-Marie-Tooth disease), cerebrotendinious xanthanomatosis, and leukodystrophies including adrenoleukodystrophy, adrenomyeloneuropathy, metachromatic leukodystrophy, globoid cell leukodystrophy (Krabbe disease), Canavan disease, vanishing white matter disease, Alexander disease, Refsum disease, and Pelizaeus-Merzbacher disease.

According to some embodiments of the invention, the demyelinating disease is multiple sclerosis (MS).

According to some embodiments of the invention, the method is effected in vivo.

According to some embodiments of the invention, the method is effected ex vivo.

According to some embodiments of the invention, the method is effected in vitro.

According to some embodiments of the invention, the agent is selected from the group consisting of a peptide agent, a small molecule agent, an antibody and a small molecule agent.

According to some embodiments of the invention, the cells are oligodendrocytes or progenitors thereof.

According to some embodiments of the invention, the cells are genetically modified to express GRP37.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-Q illustrate that Gpr37 is enriched in myelinating glia in the CNS. A-C. In situ hybridization. Sagittal-sections of adult rat brain were hybridized to GPR37 antisense probe. GPR37 mRNA is detected in white matter areas: corpus callosum (A; CC), hippocampal fimbria (B; Fi), and the cerebellar white matter tracks (C; WM). D. RT-PCR of brain RNA isolated from P12 wild type (WT) and oligodendrocyte-ablated mice (OL-). MAG and MBP were used to monitor genes that are expressed specifically in oligodendrocytes, while actin was used as a control for ubiquitously expressed genes. e. β-gal staining in a sagittal brain section of adult mice carrying a LacZ allele in the Gpr37 locus: cerebellum (cb), brain stem (bs), cerebral peduncle (cp), corpus callosum (cc), hippocampal fimbria (fi), thalamus (tl), anterior commissure (ac), optic tract (ot). F-G.

Higher magnification of the boxed areas in E. H-J. Lac Z activity monitored in whole mount preparations of optic nerve (ON), sciatic nerve (SN), and spinal cord (SC) as indicated. Representative pictures are from P70 heterozygous mice (H) and P20 (I) or P15 (J) homozygous mice. GPR37 is absent in the unmyelinated part of the optic nerve (asterisk in H), as well as in sciatic nerve (I) and spinal nerve roots (arrowheads in J) emanating from the spinal cord (asterisk in J). K-M. Immunolabeling of P12 mice caudate putamen using antibodies to βgal and Olig2. N—O. Expression of LacZ in the cerebellum (N), and optic nerve (O) isolated from P7, P12, P15 and P20 heterozygous mice. Asterisk mark the location of the white matter. P. RT-PCR analysis of GPR37 mRNA expression in mouse brain at the indicated postnatal days. Primers to actin were used as control.

The expression of GPR37 at P105 was compared to Gpr37^{-/-} (KO) mice. Q. Relative mRNA levels of GPR37 and GPR17 determined by real-time PCR analysis of whole brain RNA. Scale bars, A-C, 100 μm; E, 1 mm; F-G, 200 μm; H-J, 50 μm; K-M, 20 μm; N-O, 100 μm.

FIGS. 2A-J illustrate that the absence of Gpr37 results in faster differentiation of oligodendrocytes. A. Immunolabeling of wild type (WT) or Gpr37^{-/-} OPCs co-cultured for 3 (DIV3) or 7 (DIV7) days with DRG neurons, using antibodies to Olig2, O4, and PLP. B. The number of Olig2-positive cells (per field of view). C. Percentage of proliferating cells (labeled for ki67) at DIV3 is comparable between WT and Gpr37^{-/-} cultures. Total 2111 and 2076 cells were investigated for WT and Gpr37^{-/-}, respectively. D. Percentage of O4 positive oligodendrocytes among the total population of Olig2 positive cells (i.e., early differentiation). E. Percentage of PLP positive oligodendrocytes among the total population of O4 positive cells (i.e., late differentiation).

The number of cells already expressing PLP at DIV3 is significantly higher in Gpr37^{-/-} compared to wild type oligodendrocytes (e; * p<0.05, n=3 different cultures per each genotype at the indicated time point). F. Relative fluorescence intensity (arbitrary units) of PLP at DIV7 (* p<0.05, n=3 cultures per each genotype). G. Immunolabeling of wild type or Gpr37^{-/-} OPCs co-cultured for 9 (DIV9) days with DRG neurons, using antibodies to PLP, Caspr, and neurofilament (NF). Three different PLP-positive oligodendrocytes (label: a to c) are marked in each panel. In contrast to the wild type coculture, PLP-positive oligodendrocytes in Gpr37^{-/-} coculture are associated with intense clustering of Caspr at contact sites along the axons. H. Quantitation of the results showing the percent of PLP-positive cells that associate with axonal Caspr immunoreactivity (*P<0.05, n=3 different cultures per each genotype). I. Advanced myelination by Gpr37^{-/-} oligodendrocytes. Co-cultures (DIV9) prepared using oligodendrocytes of each genotype together with wild type mouse DRG neurons labeled with an antibody to PLP. High magnification images are shown on the right. J. The number of PLP positive myelin segments longer than 15 μm per cell is shown. Bars represent mean±standard of error (SEM) (* p<0.001, total of 100 cells each genotype were analyzed). Scale bars, A, I, 100 μm; G, 40 μm; I (insets), 20 μm. FIGS. 3A-J illustrate the absence of Gpr37 results in precocious myelination in vivo. A. Immunolabeling of WT and Gpr37^{-/-} P9 mice brains (delineated with a purple line) with an antibody to MBP. B. Higher magnification of the corpus callosum (white rectangle in a). C. Fluorescent images of longitudinal sections of optic nerves isolated from P12 PlpRed (upper panel) and PlpRed/Gpr37^{-/-} (lower panel) mice, immunolabeled with an antibody to APC (CC1). The CC1 (left) and red fluorescent (DsRed; right) signals are shown in separate panels. D. Lower magnification images showing the distribution of DsRed in PlpRed (upper panel) and PlpRed/Gpr37^{-/-} (lower panel) optic nerves. E. Fluorescence intensity of DsRed in optic nerves of the two genotypes (D) is shown per μm² (* p<0.01, n=12 sections from 4 mice per each genotype). F-G. Electron microscope images showing cross-sections through the corpus callosum of P14 WT (upper panels) and Gpr37^{-/-} (lower panels) mice. H. The number of myelinated axons per μm² was significantly higher in Gpr37^{-/-} than in WT (*p<0.05, 12 images from 3 WT and 8 images from 3 KO mice). I-J. Gpr37^{-/-} exhibit significantly thicker myelin during development. G-ratio of myelinated axons in P14 corpus callosum is presented as a function of axon diameter (I), or as an average value (J). Gpr37^{-/-} exhibit a significantly lower g-ratio than WT mice (WT=155 axons, Gpr37^{-/-}=219 axons from three mice of each genotype, *p<0.001, Student's t-test). Bars represent mean±SEM. Scale bars, A, 1 mm; B, 200 μm; C, 10 μm; D, 500 μm; F, 5 μm; G, 0.2 μm.

FIGS. 4A-I illustrate that the absence of Gpr37 results in hypermyelination of the corpus callosum. A,D,G. Electron micrographs of midsagittal sections of the corpus callosum from WT and Gpr37^{-/-} mice at the age of 2 months (A-C), 4 months (D-F) and 1.5 years (G-I). Representative higher magnification images are shown on the right columns. B, E, H. g-ratio as a function of axonal diameter. GPR37^{-/-} shows lower g-ratio than WT. Total of 274 axons (WT) and 373 axons (KO) from 3 mice (for 2 month), 481 axons (WT) and 497 axons (KO) from 3 mice (for 4 month), 97 axons (WT)and 139 axons (KO) from 2 mice (for 1.5 year) of each genotype were analyzed. C, F, I. The averaged g-ratio of Gpr37^{-/-} is significantly lower than WT (* p<0.001). Scale bars, 1 μm (left two panels) and 0.2 μm (right two panels). Bars represent mean±SEM.

FIGS. 5A-H illustrate that Gpr37-dependent inhibition of oligodendrocyte differentiation is mediated by ERK phosphorylation and nuclear translocation. A. Immunolabeling of sagittal brain sections of P12 brain stems from wild type (WT) and KO (Gpr37^{-/-}) mice using antibodies to phosphorylated ERK (pERK) and Olig2. B. The number of pERK and Olig2 positive cells in Gpr37^{-/-} compared to WT brainstem (*P<0.05, n=5 images from two mice per genotype). C. Western blot analysis of purified OPC cultures at DIV7 (three cultures per each genotype are shown). Blots were incubated with antibodies to pERK or general ERK (ERK) as indicated. D. Relative value of pERK intensity normalized by general ERK (p=0.02, n=7 cultures each genotype). E. pERK is present in the nucleus of oligodendrocytes lacking Gpr37. OPC cultures isolated from wild type (WT) or KO (Gpr37^{-/-}) mice were fixed at DIVS and immunolabeled using antibodies to pERK, PLP and Dapi. PLP and ERK immunoreactivity are shown in separate panels along with the Dapi signal. The location of the nucleus is marked with a yellow circle in the high magnification of the boxed area (insets). F. Percentage of PLP positive oligodendrocytes showing nuclear localization of pERK in wild type (WT) and KO cultures. (* p<0.05, n=3 different primary cultures for each genotype; 100 PLP-positive oligodendrocytes were counted per each culture). G-H. ERK signaling mediates Gpr37 effects on oligodendrocyte differentiation. G. OPCs isolated from Gpr37^{-/-} or WT mice as indicated were grown with wild type DRG neurons. Co-cultures were grown for one day in their growth medium and maintained for 6 days in a medium containing 10 μM EPE, 1 μM PLX4032, or DMSO as control before fixing and labeling with antibodies to Caspr and PLP. Nuclei were labeled with Dapi. H. Quantitation of the results showing the fold change in the percent of PLP-positive cells that associate with axonal Caspr immunoreactivity in all samples compare to the non-treated wild type cultures. Gpr37^{-/-} oligodendrocytes treated with ERK inhibitors showed a significantly low number of Caspr-positive axons, when compared to DMSO-control (*P<0.05, **P<0.01, ***P<0.001; n=3 different primary cultures for each genotype, 100 PLP-positive cells were counted in each treatment). Bars represent mean±SEM. Scale bars, a, 100 μm; e, 50 μm h, 20 μm.

FIGS. 6A-B illustrate how GPR37 regulates oligodendrocyte myelination through MAPK signaling. A. Sequential expression and activity of GPCRs during differentiation of the oligodendrocyte lineage. GPR56 regulates OPC proliferation, while GPR17 and GPR37 negatively regulate two consecutive stages of oligodendrocytes differentiation. B. A schematic model depicting GPR37 signaling. Relief of GPR37 inhibition results in increase in cAMP and Epac-dependent activation of MAPK cascade, resulting in translocation of phospho-ERK1/2 into the nucleus and myelination. Red lines mark the point of action of the various pharmacological inhibitors used.

FIGS. 7A-E illustrate that expression of GPR37 and GPR17 is mutually exclusive. A-C. Immunolabeling of P12 PlpRed mouse brainstems with an antibody to GPR37 and GPR17. Three representative pictures are shown. Arrows indicate cells expressing both GPR37 and PLPdsRed, while open triangles indicate cells expressing GPR17. Scale bars, 10 μm. D. Percentage of GPR17- and GPR37-positive cells among the total cell population expressing either GPR17 or GPR37 (n=204 cells from three different mouse brains). Only 4% of the cells were expressing both GPCRs. E. Percentage of cells expressing GPR17 or GPR37 among the total population of PLPdsRed-positive cells (n=61 cells from three different mouse brains).

FIGS. 8A-D illustrate that shRNA knockdown of GPR37 enhances oligodendrocyte differentiation. A. qPCR analysis of OPC cultures at DIVS. Expression of GPR37 in OPC cultures infected with retroviruses containing pSuper-shRNA vectors (shRNA1 or shRNA2 or combined ‘sh1+2’), compared to wild type (WT), vector alone (Con) or OPCs isolated from Gpr37^{-/-} mice. ns, not significant. B. Percentage of PLP positive oligodendrocytes among the total population of O4 positive cells in empty vectors (Con)- or combined shRNA vectors (sh)-infected OPCs co-cultured with DRG neurons. The number of cells expressing PLP at DIV3 is significantly higher in GPR37 knockdown oligodendrocytes (**P<0.01, n=3 cultures, t-test). Bars represent mean±SEM. C-D. Immunostaining of infected OPCs co-cultured with DRG neurons at DIV3, using antibodies to PLP, 04 and GFP (GFP was used to mark infected cells). D. Higher magnification of shRNA-infected cocultures showing the increase in PLP expression in a cell expressing high (A), but not low (B) levels of GFP. Scale bar, 50 μm (C), 20 μm (D).

FIGS. 9A-B illustrate that the absence of Gpr37 does not affect OPC proliferation. A. Longitudinal sections of optic nerves isolated from wild type (WT) and Gpr37^{-/-} (KO) mice at P9, P12, and P86 were immunolabeled using Ki67 and PDGFRa antibodies. The graph shows the percentage of proliferating OPCs (Ki67/PDGFRa) at the different ages. The following number of sections from 2 mice of each genotype was used for analyses of each age (P9: WT=9, KO=8; P12: WT=10, KO=10; P86: WT=11, KO=12). B. Number of PDGFRa cells present in P12 optic nerves isolated from Gpr37^{-/-} mice and their littermate controls per field of view (FOV). Ten images (magnification 63×) from two mice per genotype were analyzed. Bars represent mean±SEM.

FIGS. 10A-I illustrate that the absence of Gpr37 resulted in thicker myelin in the spinal cord. Quantification of myelin thickness was done by analyzing electron microscope images of spinal cords isolated from wild type (WT) and Gpr37^{-/-} (KO) mice at the age of 2 months (A-C), 4 months (D-F) and 1.5 years (G-I). A,D,G. g-ratio is presented as a function of the corresponding axon diameter. Total of 210 axons (WT) and 223 axons (KO) from 2 mice (for 2 month), 438 axons (WT) and 334 axons (KO) from 4 WT mice and 3 KO mice (for 4 month), 206 axons (WT) and 315 axons (KO) from 2 mice (for 1.5 year) of each genotype were examined for the analysis. B, E, H. The averaged g-ratio is significantly lower in GPR37^{-/-} than WT (*P<0.001, t-test). C, F, I. Averaged axonal diameter is not significantly changed in spinal cord of Gpr37^{-/-} and WT. Bars represent mean±SEM.

FIGS. 11A-B illustrate that EPE blocks the nuclear translocation of pERK in Gpr37 null oligodendrocytes. A. Cultured OPCs isolated from WT and Gpr37^{-/-} mice were grown for 3 days (DIV2-DIVS) in the presence or absence (control) of EPE, followed by immunolabeling with antibodies to pERK and PLP. The nuclei were labeled with Dapi. PLP and pERK immunoreactivity are shown in separate images along with the Dapi signal. Higher magnification of the nucleus is shown in the insets in each panel. EPE prevented the nuclear translocation of pERK seen in Gpr37^{-/-} oligodendrocytes. Scale bar, 50 μm. B. ERK inhibitors do not affect the number of oligodendrocyte co-cultured with DRG neurons. Cultures were grown in the absence (DME), or presence of EPE, PLX4032, or DMSO as control for 6 days, and then fixed at DIV7 and labeled with antibodies to Olig2. The number of positive cells per field of view is shown in the graph. Bars represent mean±SEM (n=3 different cultures per each genotype; 10 fields of view were analyzed for each culture).

FIGS. 12A-D illustrate the involvement of cAMP in Gpr37 signaling in oligodendrocytes. A-B. Absence of Gpr37 results in increased levels of cAMP in mice brain (A) and cultured OPCs (B). Intracellular cAMP was extracted from P70 mice brains (*P<0.05, t-test; n=6 WT and n=7 KO mice), or from DIV5 cultured OPCs (*P<0.01, t-test; n=3 different OPC primary cultures of each genotype) and detected by ELISA. C. Adenylate cyclase inhibition prevents the nuclear translocation of pERK in Gpr37^{-/-} OPCs. Cultured wild type (WT) and Gpr37^{-/-} OPCs (DIV7) were left untreated (control) or treated with the adenylyl cyclase inhibitor SQ22536 (0.1 mM) for 2 hours in their growth medium. Cells were then fixed and labeled with Dapi to mark their nuclei, and with antibodies to PLP and pERK. PLP and pERK immunoreactivity are shown in separate panels along with the Dapi signal. Insets show higher magnification of the boxed area in each panel. Scale bar: 50 μm. D. Quantification of the immunolabeling, showing that the nuclear localization of pERK in Gpr37^{-/-} oligodendrocytes was reduced to equivalent levels of wild type cells after the treatment. Bars show mean±SEM (*P<0.01, t-test; n=3 different primary cultures for each genotype; 50 PLP-positive cells were counted per each culture).

FIGS. 13A-B illustrate that inhibition of Epac activity attenuates nuclear translocation of ERK1/2 in oligodendrocytes lacking GPR37. A. Cultured wild type (WT) and Gpr37^{-/-} OPCs (DIV7) were treated with DMSO (Control) or with the Epac inhibitor ESI-09 (1 μM) for 2 hours in their growth medium. Cells were then fixed and labeled with Dapi to mark their nuclei, and with antibodies to PLP and phosphorylated ERK (pERK). PLP and pERK immunoreactivity are shown in separate panels along with the Dapi signal. Insets show higher magnification of the boxed area in each panel. Scale bar: 40 μm. B. Quantification of the immunolabeling, showing that the nuclear localization of pERK in GPR37^{-/-} oligodendrocytes was reduced to equivalent level of wild type cells after the treatment. Bars show mean±SEM (*P<0.05, **P<0.01, t-test; n=3 different primary cultures for each genotype; 100 PLP-positive cells were counted per each culture).

FIGS. 14A-B illustrate that cAMP enhances oligodendrocyte differentiation. A. Immunolabeling of wild type OPC cultures grown for four days in the absence (Control) or presence of 1mM 3-isobutyl-1-methylxanthine (IBMX) using antibodies to PLP and O4. Dapi was used to label the nuclei. Scale bar, 50 μm. B. Percentage of PLP-positive oligodendrocytes among the total O4-positive cells (**P<0.01, t-test, n=3 cultures). Bars represent mean±SEM.

FIG. 15 illustrates that the absence of GPR37 results in an increase in Myrf and a decrease in Hes5 expression. qRT-PCR analysis of P4 brainstems isolated from wild type (WT) and GPR37^{-/-} (KO) mice. Gene expression levels of the other indicated transcription factors do not show a significant change. Bars represent mean±SEM, WT=3 mice, KO=6 mice; *P=0.01, t-test.

FIGS. 16A-B illustrate the effect of prosaptide on oligodendrocyte differentiation. A. OPC/DRG neuron cocultures were grown with the indicated concentration of prosaptide (ANASPEC) for three days. Cells were then labeled with antibodies to PLP and O4. Dapi was used to stain the nuclei. Scale bar, 100 μm. B. Percentage of PLP-positive oligodendrocytes among the total cell populations expressing O4 is shown. *P<0.05, **P<0.01, t-test, n=3 cultures per each genotype at each concentration. Bars represent mean±SEM. n.s., not significant.

FIG. 17 illustrates original Western blots of OPC cultures using antibodies to phosphorylated (pERK) and general ERK (ERK). Three different OPC cultures from wild type (WT) and Gpr37^{-/-} (KO) mice are shown along with the location of molecular mass markers in kDa. The framed area is shown in FIG. 5C.

FIG. 18 is a graph illustrating that mice lacking Gpr37 exhibit a faster recovery after EAE as compared to WT mice.

FIGS. 19A-B are electron microscopy micrographs of wild type and Gpr37 KO spinal cord 19 days after the induction of EAE portraying massive demyelination and axonal loss in WT but not in the KO (which likely underwent complete remyelination) Scale bar 5 um.

FIGS. 20A-G illustrate that lysolecithin induces demyelination in both WT and Gpr37^{-/-} mouse corpus callosum. A, Representative images of WT (upper) and Gpr37^{-/-} (lower) corpus callosum at 3 (left) and 12 (right) day post lesion (dpl). MBP (green) identifies myelin protein and dapi (blue) identifies cell nuclei. B-C, Quantification of Dapi+ cell numbers and MBP density in the intact (left) and the lesion (right) area at 3 dpl. D, Size of the lesion area (dapi-accumulated area) at 3 dpl. E-F, Quantification of Dapi+ cell numbers and MBP density in the intact (left) and the lesioned (right) area at 12 dpl. G, Size of the lesion area (dapi-accumulated area) at 12 dpl. (B,C,E,F) For statistical analysis, at least three images (magnification 40×) per mouse (at least three mice per genotype at each time point) were randomly acquired from the center of the lesion area as well as the intact area. The area size for the analysis was 160.71 μm×160.71 μm. (D,G) For statistical analysis, at least three images (magnification 10×0.5×) were acquired from at least three mice per genotype at each time point. Error bars represent mean±SEM. ***p<0.001; significance based on Student's t test. N=9-15 (More than 3 images per mouse were pooled from more than 3 mice per genotype at each time point). Scale bar=100 μm.

FIGS. 21A-D illustrate that the number of OL-lineage cells in early stage is not significantly different in both lesion sites. A, Representative images, immunostained for Olig2(red)/Dapi(blue)(left) and PDGFRa(green)/GPR17(red)/Dapi(blue)(right), were taken randomly from the middle of the lesion of WT (upper) and Gpr37^{-/-} (lower) corpus callosum at 3 dpl. Scale bar=10 μm. B-D, Quantification of Olig2+ cell numbers (B), PDGFRa+ cell numbers (C) and GPR17+ cell numbers (D) at 3 dpl. For statistical analysis, at least three images (magnification 40×) per mouse (at least three mice per genotype) were randomly acquired from the center of the lesion area as well as the intact area. The area size for the analysis was 160.71 μm×160.71 μm. Error bars represent mean±SEM.

FIGS. 22A-D illustrate that the level of remyelination is higher in KO compare to WT brains. A, Lesioned area of WT (left) and Gpr37^{-/-} (right) corpus callosum at 12 dpi stained PLP (green) and Dapi (blue). Lesions were identified by the accumulated nuclei (Dapi, blue) and outlined (red dot line). Scale bar=100 μm. B, Quantification of PLP density at 12 dpi. C, Representative images, immunostained for MBP (green)/Dapi (blue), taken from the lesion of WT (left) and Gpr37^{-/-} (right) corpus callosum at 12 dpi. D, Quantification of MBP density at 12 dpi. For statistical analysis, ten single stack images per mouse were investigated from at least three mice per genotype (N=30-40). Error bars represent mean±SEM. ***p<0.001; significance based on Student's t test.

FIGS. 23A-E illustrate that the number of Caspr positive clusters per area is significantly higher in Gpr37^{-/-} compared to WT lesion. A-D, Representative images, immunostained for Caspr (green)/Dapi (blue), were taken randomly from the lesion area of WT (A,B) and Gpr37^{-/-} (C,D) corpus callosum at 12 days post injection (dpi). A,C, Lesions were identified by the accumulated nuclei (Dapi, blue) and outlined (red dot line). B,D, High-magnification images were acquired from the center of the lesion area. E, Quantification of Caspr+ dot numbers per area. For statistical analysis, at least three images (magnification 40×) per mouse (at least three mice per genotype) were randomly acquired from the center of the lesion area. The area size for the analysis was 160.71 μm×160.71 μm. Error bars represent mean±SEM. *p<0.05; significance based on

Student's t test. N=9. Scale bars=100 μm (A,C), 10 μm (B,D).

FIGS. 24A-C illustrate that the number of early-stage oligodendrocytes (PDGFRa+) is significantly higher in WT lesion area compared to KO lesion area. A-B, Representative images, immunostained for PDGFRa (green)/Dapi (blue), were taken randomly from the lesion area of WT and Gpr37^{-/-} corpus callosum at 12 days post injection (dpi). A, Lesions were identified by the accumulated nuclei (Dapi, blue) and outlined (red dot line). B, High-magnification images were acquired from the center of the lesion area. C, Quantification of PDGFRa+ cell numbers per area. For statistical analysis, at least three images (magnification 40×) per mouse (at least three mice per genotype) were randomly acquired from the center of the lesion area. The area size for the analysis was 160.71 μm×160.71 μm. Error bars represent mean±SEM. ***p<0.001; significance based on Student's t test. N=9. Scale bars=100 μm (A), 10 μm (B).

FIGS. 25A-B illustrate that the absence of Gpr37 prevents the chronic progression of EAE pathology. (A) EAE disease scores in WT and Gpr37^{-/-} mice injected with encephalitogenic MOG peptide. The scores of each genotype are significantly different (*p<0.05, Student's t test) from day 18 until the end of the experiment. EAE was scored as follows: 0—no disease, 1—limp tail, 2—hind limb paralysis, 3—paralysis of all limbs, 4—moribund condition, and 5—death. (B) The area under the curve (AUC) is calculated from the graph (A). *p<0.05, Student's t test (N=14 (WT) & 20 (KO)).

FIGS. 26A-G illustrate that the lack of Gpr37 reduces CNS inflammation in EAE. (A_F) Representative H&E staining pictures of lumbar spinal cords of WT (left) or Gpr37^{-/-} (right) mice in EAE model at day 30 after the MOG injection. (B,E) Higher magnification of the ventral-lateral column of the spinal cord of WT or Gpr37^{-/-} mice (black dotted rectangles in A and D). (C,F) Higher magnification of the ventral-lateral column of the spinal cord of WT or Gpr37^{-/-} mice (red dotted rectangle in B and F). In WT tissues, there are obviously more number of nucleus (stained as purple dots) and tissue damages (white bubble-like structures in the tissue) than in Gpr37^{-/-} tissues. Scale bars, 100 μm. (G) Quantification of the number of inflammatory foci (H&E staining) in the whole area of the lumbar spinal cord at day 12, 15, 19 and 30 after the MOG injection. Note the significant increase in the number of nucleus between day 12 and day 19 in both WT and Gpr37^{-/-} tissues. However, the dynamics in inflammation is different between the genotypes after day 19. In Gpr37^{-/-} tissues, the number of nucleus was reduced dramatically by day 30, compared to day 19, while no significant changes were observed in WT tissues. *p<0.05, **p<0.01, ***p<0.001, Student's t test (N=average 11 sections pooled from average 3 mice per genotype per time point).

FIGS. 27A-D illustrate that there is an increase in myelin and decrease in inflammation in the lumbar spinal cords of Gpr37^{-/-} compared to WT at day 30 after MOG injection. (A-B) Lumbar spinal cords are stained with myelin (MBP, green), microglia/macrophage (Iba, red) and astrocyte (GFAP, blue) markers. (B) Higher magnification of the ventral-lateral column of the spinal cord of WT or Gpr37^{-/-} mice (white rectangles in A). Scale bars, 100 μm. (C) Quantification of MBP-positive (MBP⁺) % Area in the white matter of the lumbar spinal cord at day 12, 15, 19 and 30 after the MOG injection. From day 19 till day 30, there were significant reductions in MBP % Area in the lumbar spinal cords of WT, while LFB⁺ % Area in Gpr37^{-/-} tissues stayed unaltered. The intensity of MBP in actively regenerating myelin is higher than in pre-existing myelin, resulting in mild changes in MBP intensity dynamics in Gpr37^{-/-} tissues. However, there is a dramatic difference in MBP % Area between genotypes at day 30. *p<0.05, **p<0.01, Student's t test (N=average 7 sections pooled from average 3 mice per genotype per time point). (D) Quantification of Iba⁺ % Area in the white matter of the lumbar spinal cord at day 12, 15, 19 and 30 after the MOG injection. There is a continuous increase of Iba⁺ % Area in WT lumbar spinal cords throughout the whole investigated periods, while there is no significant changes of Iba⁺ % Area in Gpr37^{-/-} tissues. These gaps result in significant changes in Iba⁺ % Area between WT and Gpr37^{-/-} at day 19 and 30. **p<0.01, ***p<0.001, Student's t test (N=average 7 sections pooled from average 3 mice per genotype per time point). n.d., no data.

FIGS. 28A-C illustrate the mitigation of activated microglia population in the lumbar spinal cords of Gpr37^{-/-} compared to WT. (A-B) Lumbar spinal cords at day 30 are stained with activated microglia (Mac2) marker. (B) Higher magnification of the ventral-lateral column of the spinal cord of WT or Gpr37^{-/-} mice (white rectangles in A). Scale bars, 100 μm. (C) Quantification of Mac²⁺ % Area in the white matter of the lumbar spinal cord at day 12, 15, 19 and 30 after the MOG injection. In the white matter of the lumbar spinal cord of WT, there is a continuous increase of Mac2⁺ % Area throughout the entire observation period. In Gpr37^{-/-} tissues, there is no significant changes during the investigated period, resulting in significant differences in Mac2+ % Area at day 19 and 30 between WT and Gpr37^{-/-}. *p<0.05, **p<0.01, Student's t test (N=average 8 sections pooled from average 3 mice per genotype per time point).

FIGS. 29A-C illustrate the mitigation of T cell accumulation in the lumbar spinal cords of Gpr37^{-/-} compared to WT. (A) Lumbar spinal cords are stained with T cell (CD3, red), myelin (MBP, green) and myelinating oligodendrocytes (Ermin, blue) markers. (B) Higher magnification of the ventral-lateral column of the spinal cord of WT or Gpr37^{-/-} mice (white rectangles in A). Scale bars, 100 μm. (C) Quantification of CD3⁺ cell number in the white matter of the lumbar spinal cord at day 12, 15, 19 and 30 after the MOG injection. In the white matter of the lumbar spinal cord of WT, there is a continuous increase of CD3⁺ cell number during day 12-19 and the cell number stays unaltered till the end of the observation. In Gpr37^{-/-} tissues, there is an increase in CD3⁺ cell number during day 12-15 and it stays unaltered till day 19. However, there is a dramatic reduction in CD3⁺ cell number during 19-30. *p<0.05, **p<0.01, ***p<0.001, Student's t test (N=average 8 sections pooled from average 3 mice per genotype per time point).

FIGS. 30A-G illustrate the extensive remyelination in the absence of Gpr37. Lumbar spinal cord section were stained with Luxol fast blue (LFB) (A-F) and nuclei are shown by haematoxylin and eosin (H&E) staining in purple (A, D) at day 30 after the MOG injection. (B, E) Myelin staining of lumbar spinal cords of WT and Gpr37^{-/-} show higher myelin recovery in Gpr37^{-/-} compared to WT at day 30. Tissues are outlined with yellow line. (C, F) Higher magnification of the ventral-lateral column of the spinal cord of WT or Gpr37^{-/-} mice (red dotted rectangles in B and E). Scale bars, 100 μm. (G) Quantification of LFB-positive (LFB⁺) % Area in the white matter of the lumbar spinal cord at day 12, 15, 19 and 30 after the MOG injection. From day 15 till day 19, there were significant reductions in LFB⁺ % Area in the lumbar spinal cords of both genotypes, indicating the robust demyelination during this period in both genotypes.

However, LFB⁺ % Area in Gpr37^{-/-} tissues was significantly increased from day 19 till day 30, while LFB⁺ % Area in WT tissues stayed unaltered, suggesting the extensive remyelination under the absence of Gpr37. **p<0.01, ***p<0.001, Student's t test (N=average 15 sections pooled from average 3 mice per genotype per time point).

FIGS. 31A-C illustrate the extensive remyelination in the lumbar spinal cords of Gpr37^{-/-} but not in WT. (A-B) Lumbar spinal cords are stained with myelinating oligodendrocyte marker Opalin (Tmem10). (B) Higher magnification of the ventral-lateral column of the spinal cord of WT or Gpr37^{-/-} mice (white rectangles in A). Scale bars, 100 μm. (C) Quantification of Opalin % Area in the white matter of the lumbar spinal cord at day 12, 15, 19 and 30 after the MOG injection. In WT tissues, there is a significant reduction in Opalin⁺ % Area during day 12-15 and the level of Opalin⁺ % Area stays in low level till the end of observation. In Gpr37^{-/-} tissues, there is a gradual reduction in Opalin⁺ % Area till day 19. However there is a dramatic increase in Opalin⁺ % Area during day 19-30. *p<0.05, ***p<0.001, Student's t test (N=average 12 sections pooled from average 3 mice per genotype per time point).

FIGS. 32A-L illustrate the toluidine blue staining of semi-thin sections to visualize remyelination and demyelinated areas in the lumbar spinal cords of WT-EAE or Gpr37^{-/-}-EAE animals at day 30. (A, G) Representative pictures of ventral-lateral column of the spinal cord of WT or Gpr37^{-/-} mice. (B, H) Higher magnification of the ventral column of the spinal cord of WT or Gpr37^{-/-} mice (white dotted rectangles in A, G). (C, D, I, J) Higher magnification of the ventral column of the spinal cord of WT or Gpr37^{-/-} mice (white dotted rectangles in B, H). (E, K) Higher magnification of the ventral-lateral column of the spinal cord of WT or Gpr37^{-/-} mice (white dotted rectangles in A, G). (F, L) Higher magnification of the ventral-lateral column of the spinal cord of WT or Gpr37^{-/-} mice (white dotted rectangles in E, K). WT tissues show more damages such as empty spaces (white) and demyelinated areas (without ring-like structures) compared to Gpr37^{-/-} tissues. The closer to the surface of the spinal cord, the greater the differences of damage level between WT and Gpr37^{-/-} tissues. Scale bars, 50 μm.

FIGS. 33A-F. Electron microscopic visualization of remyelination after day 30 in EAE at the level of lumbar spinal cords. (A-B) Low-magnification electron micrographs show demyelination as well as abnormal myelin area near the surface of ventral-lateral spinal cord of WT (A) and Gpr37^{-/-} (B) mice. (C) High-magnification electron micrographs showing remyelinated axons (red asterisks) in Gpr37^{-/-} mice (white dotted rectangle in B). Cell bodies of oligodendrocytes are indicated (#). (D) Higher magnification of c (white dotted rectangles in C). Remyelinated axons with thin axons are labeled (red asterisks). (E) Higher magnification of D (white dotted rectangles in D).

Remyelinated axons with thin axons are labeled (red asterisks). (F) High-magnification electron micrographs showing remyelinated axons (red asterisk). Cytoplasm of an uncertain cell type near the remyelinated axon is labeled (white asterisk). Scale bars, 5 μm (A-C), 1 (D), 500 nm (E-F).

### DESCRIPTION OF SPECIFIC EMBODIMENTS OF THE INVENTION

The present invention, in some embodiments thereof, relates to methods of modulating neuronal myelination using agents that target G Protein-Coupled Receptor 37 (GPR37) and, more particularly, but not exclusively, to use of the agents for treating diseases associated with aberrant myelination.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Destruction of myelin in the CNS not only leads to devastating white matter diseases such as leukodystrophies and Multiple Sclerosis, but is also associated with psychiatric disorders, and neurodegenerative diseases. Hence, identification of agents that promote oligodendrocyte development and myelination, as well as their maintenance and ability to remyelinate after damage, is of great clinical interest.

Whilst reducing the present invention to practice, the present inventors have uncovered that GPR37 is a negative regulator of oligodendrocyte maturation (i.e., late stage differentiation) and myelination and suggest use of agents that inhibit GPR37 for the treatment of myelinating disorders.

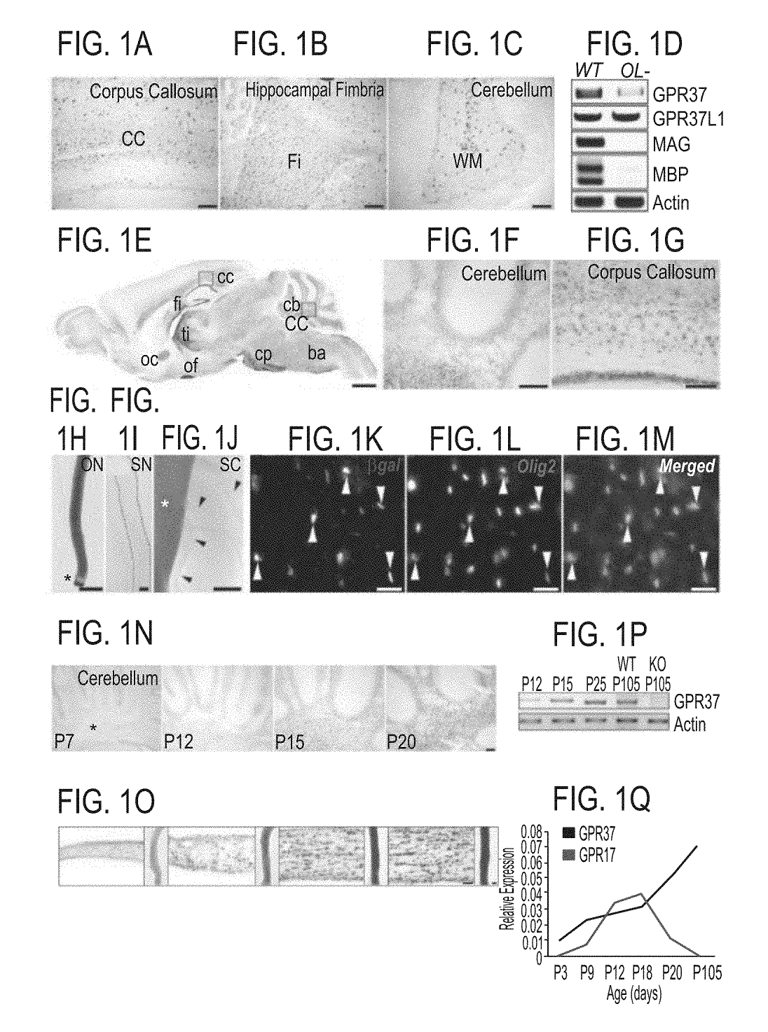

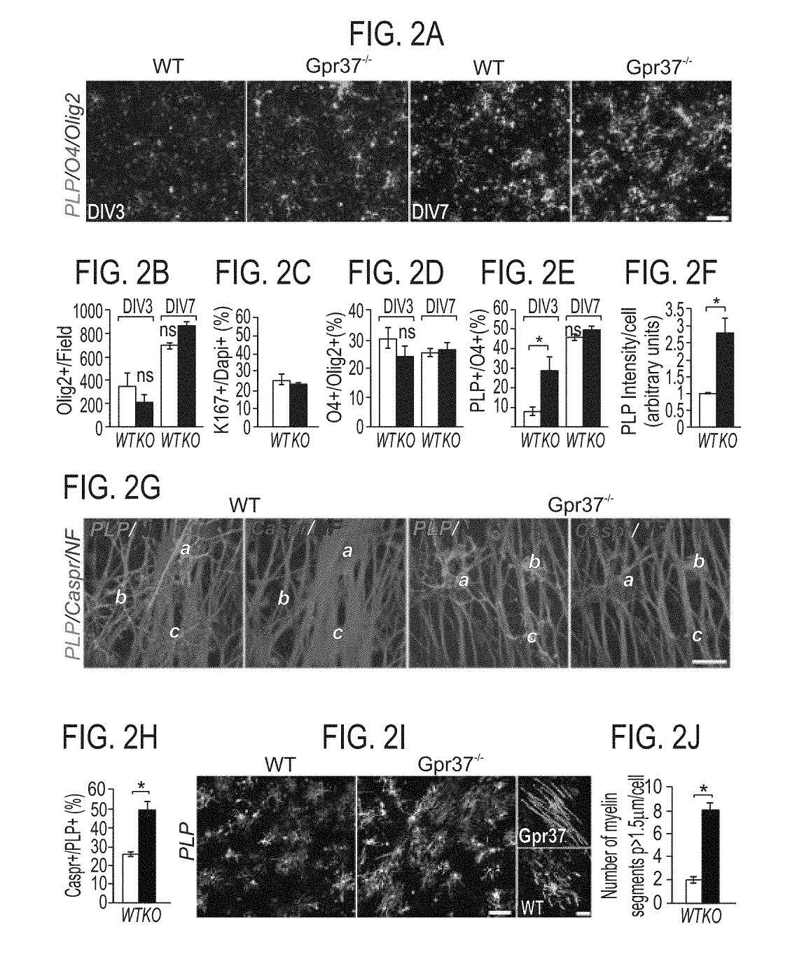

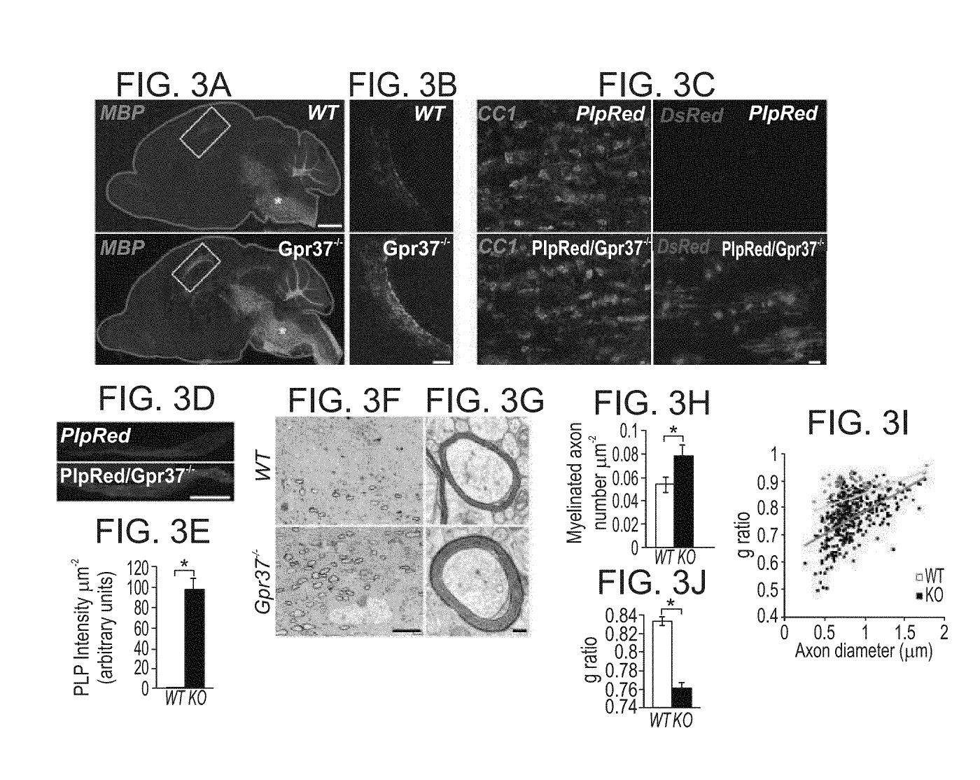

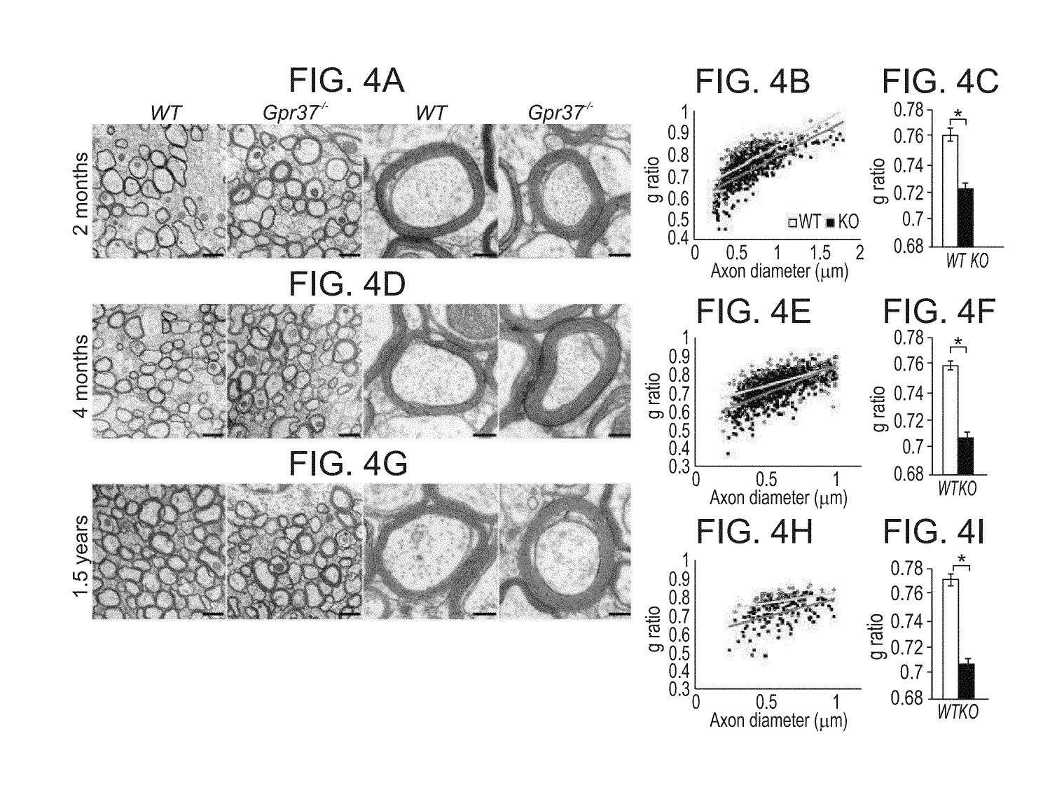

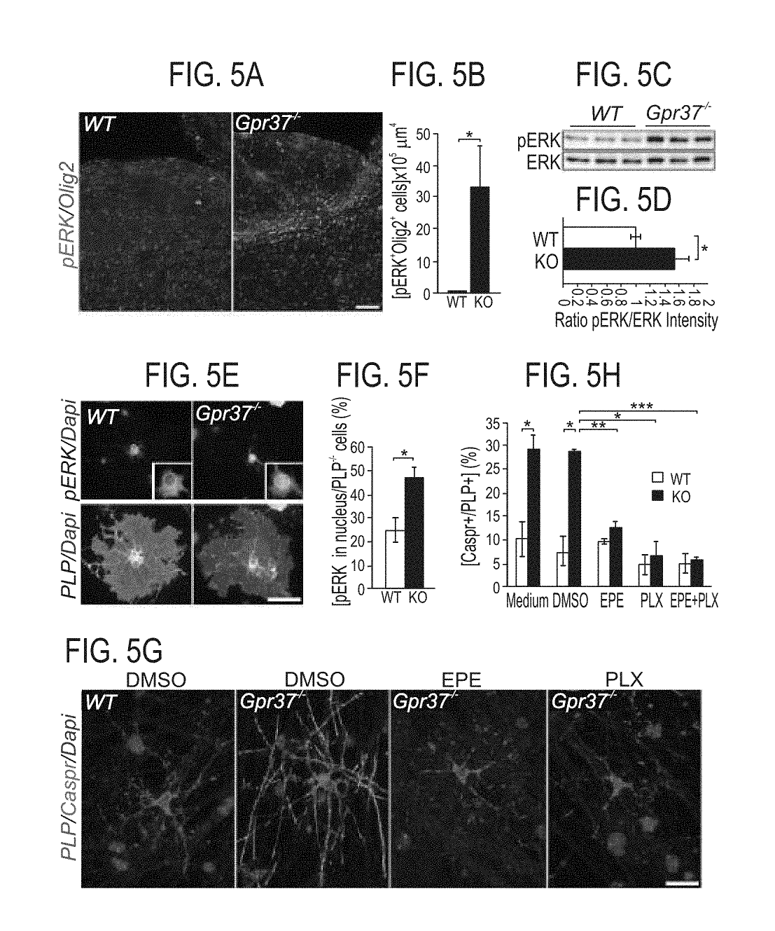

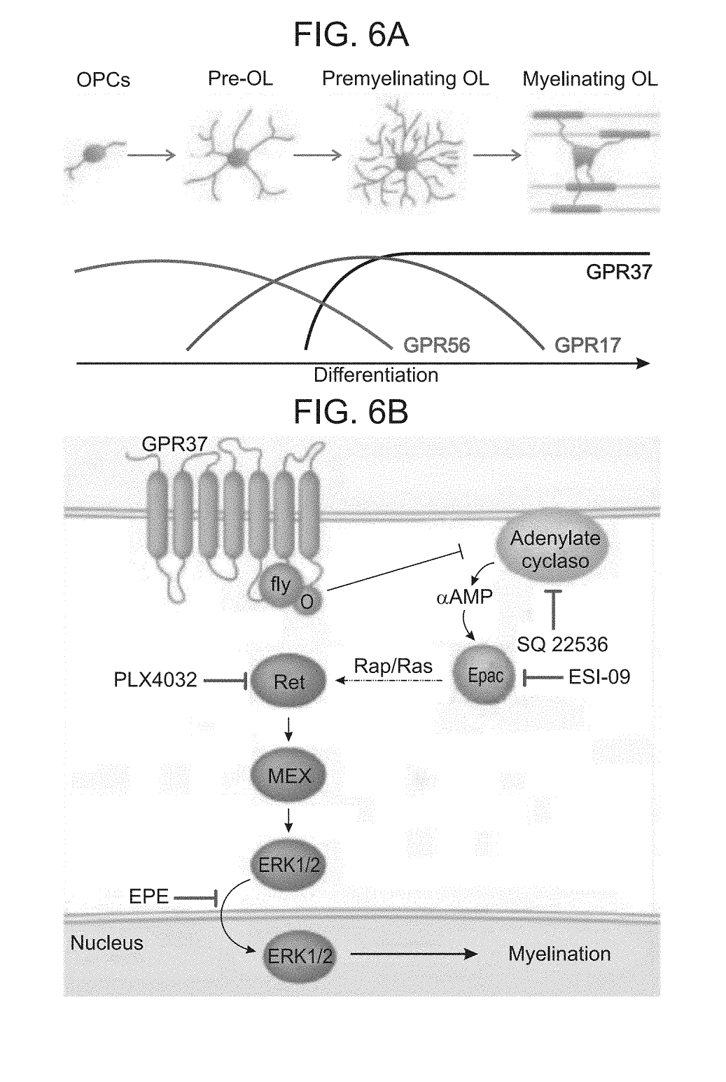

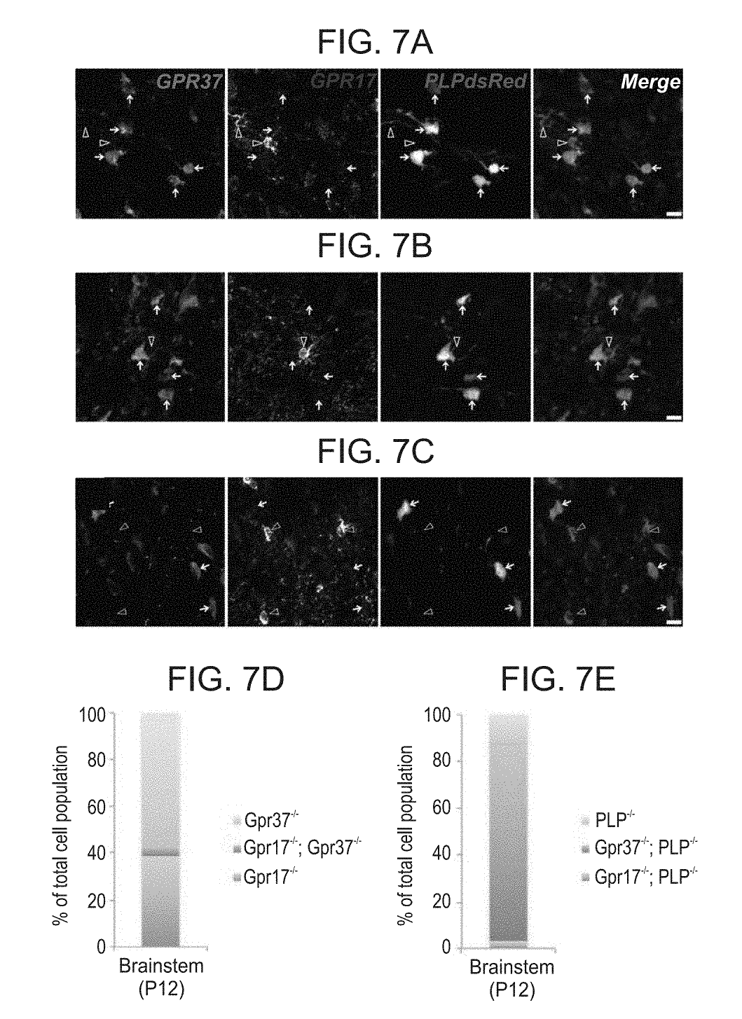

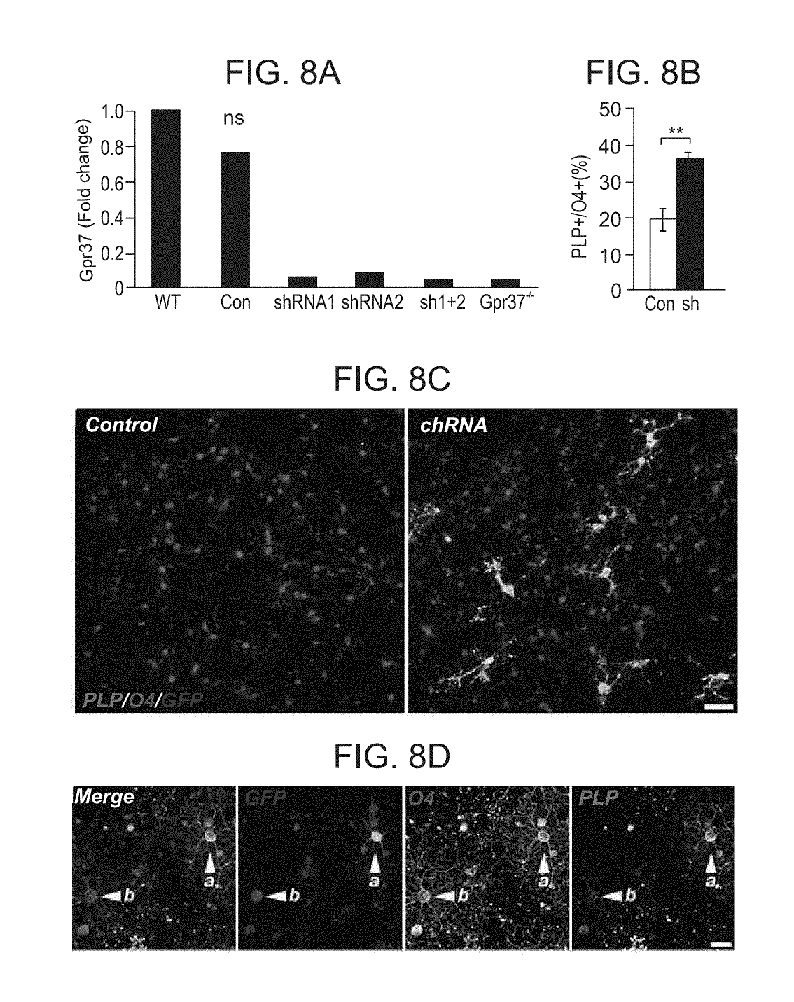

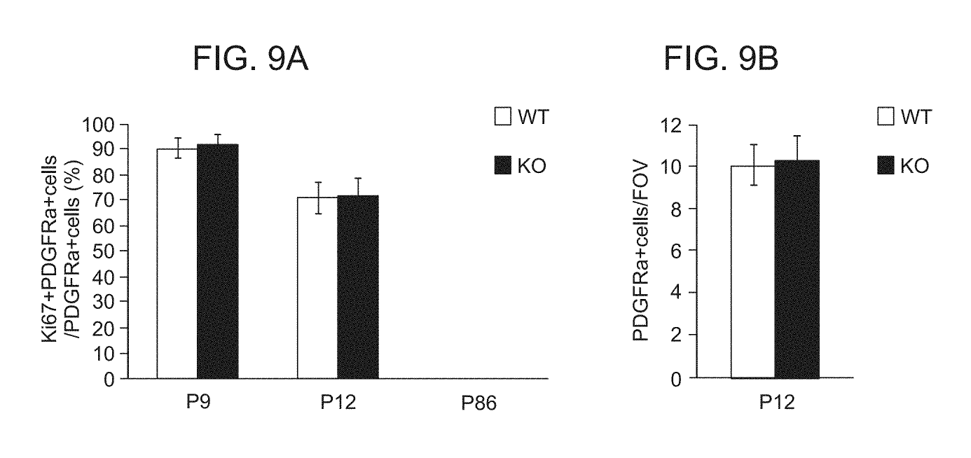

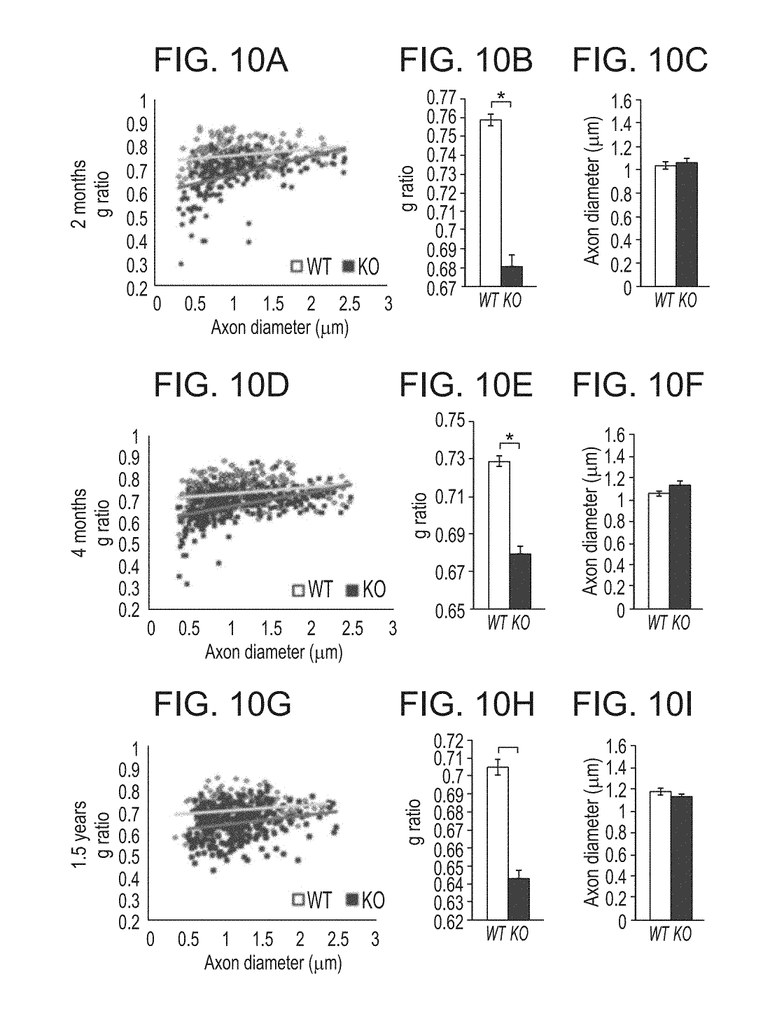

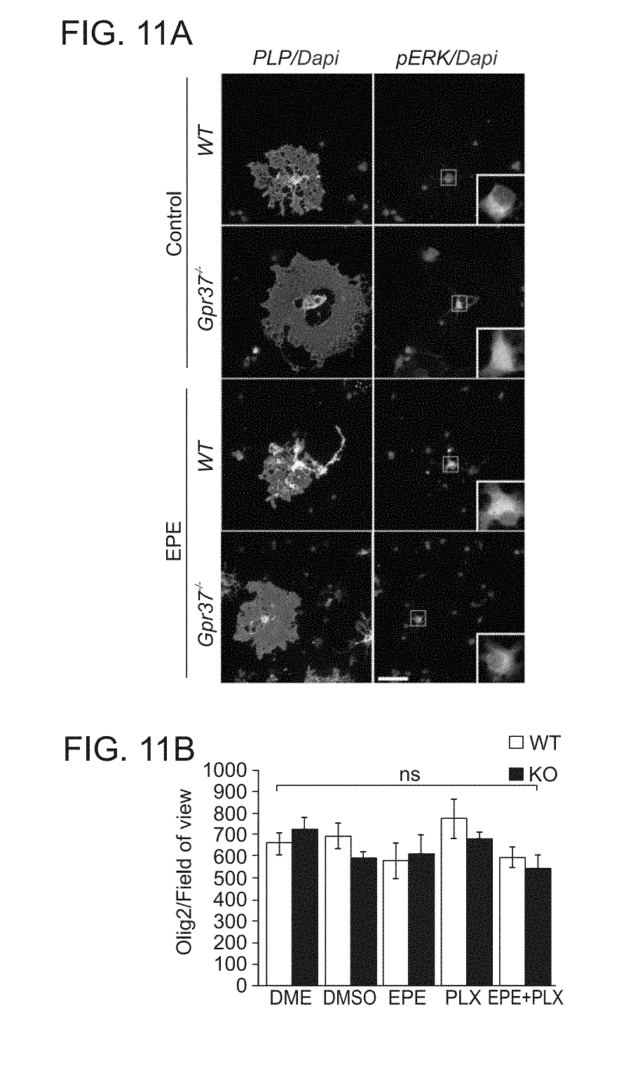

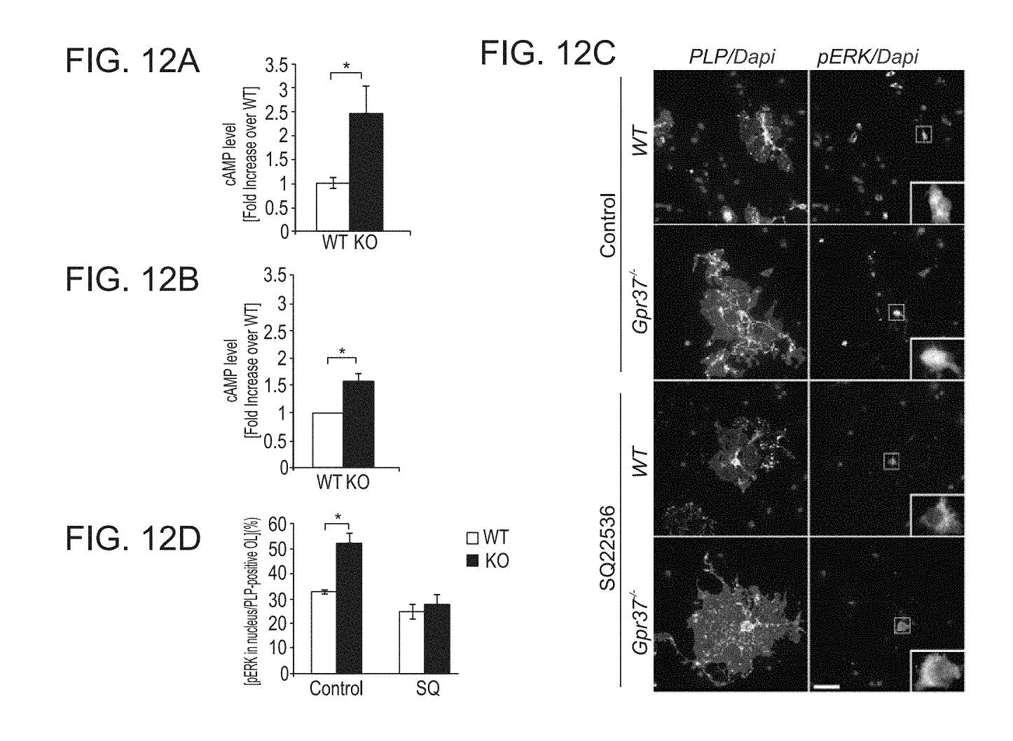

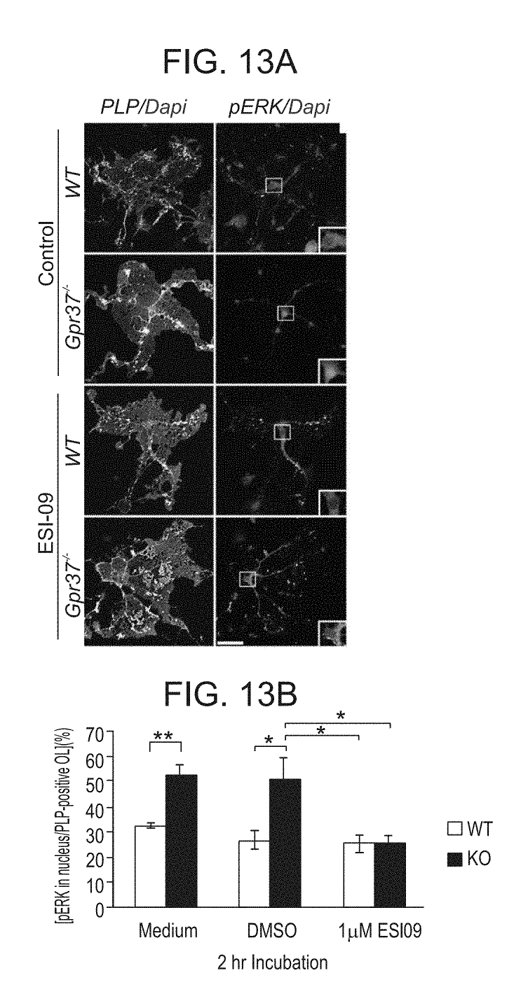

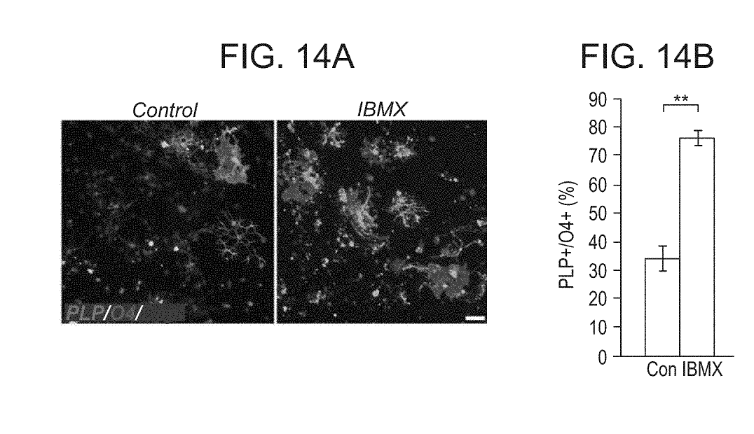

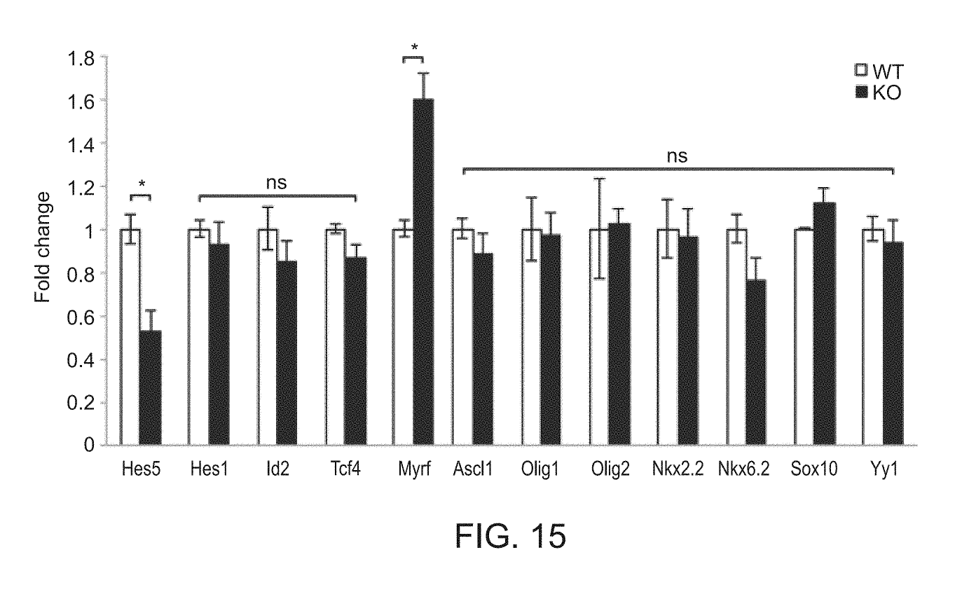

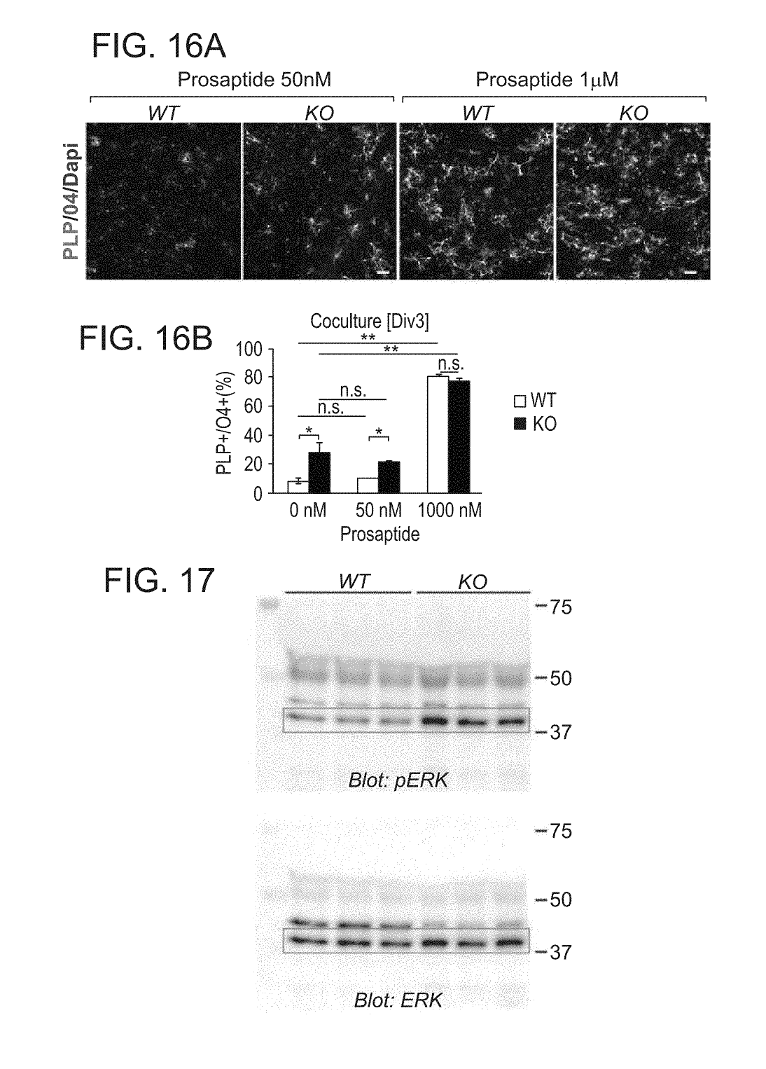

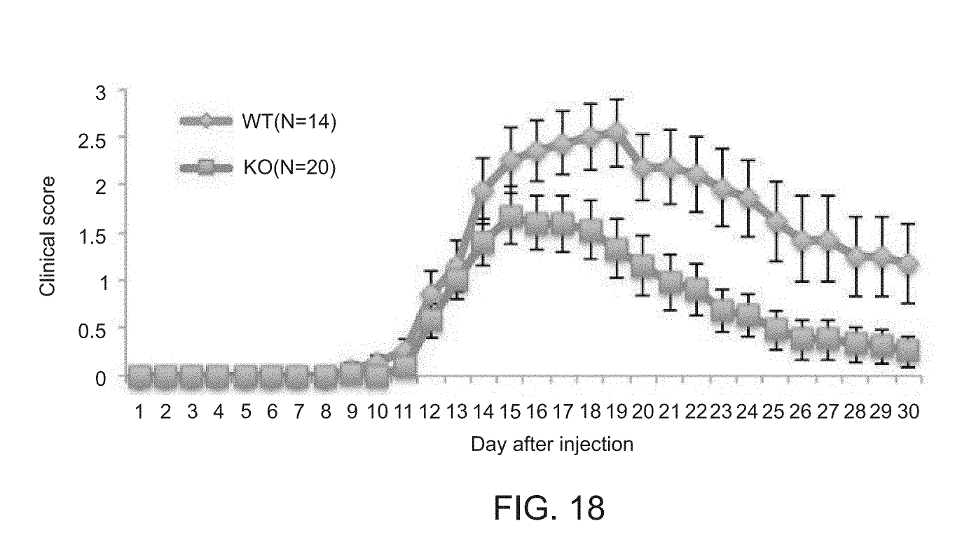

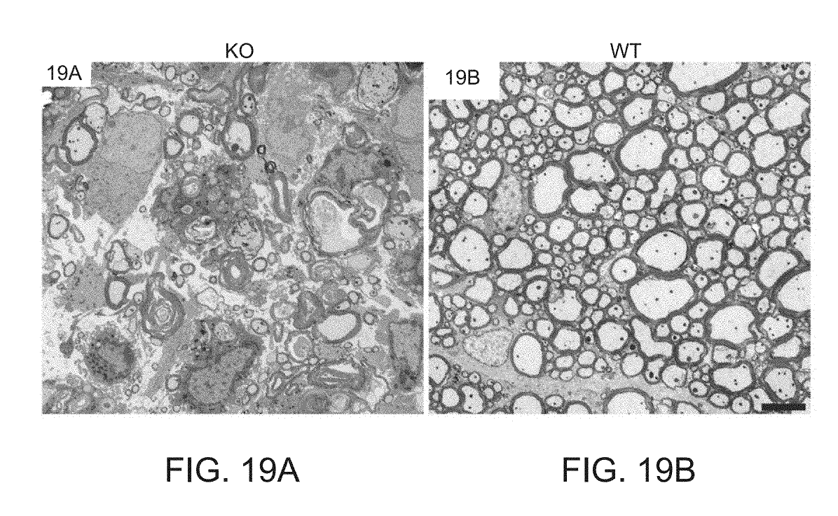

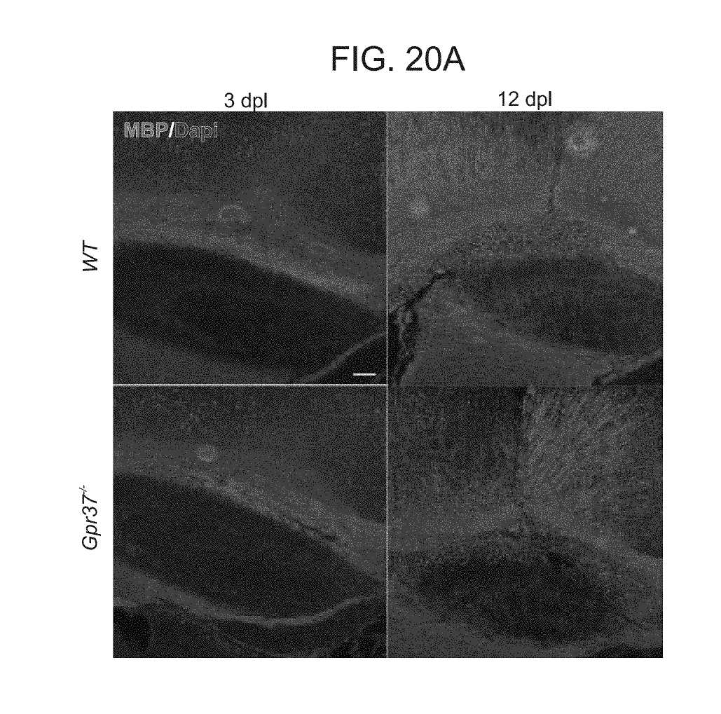

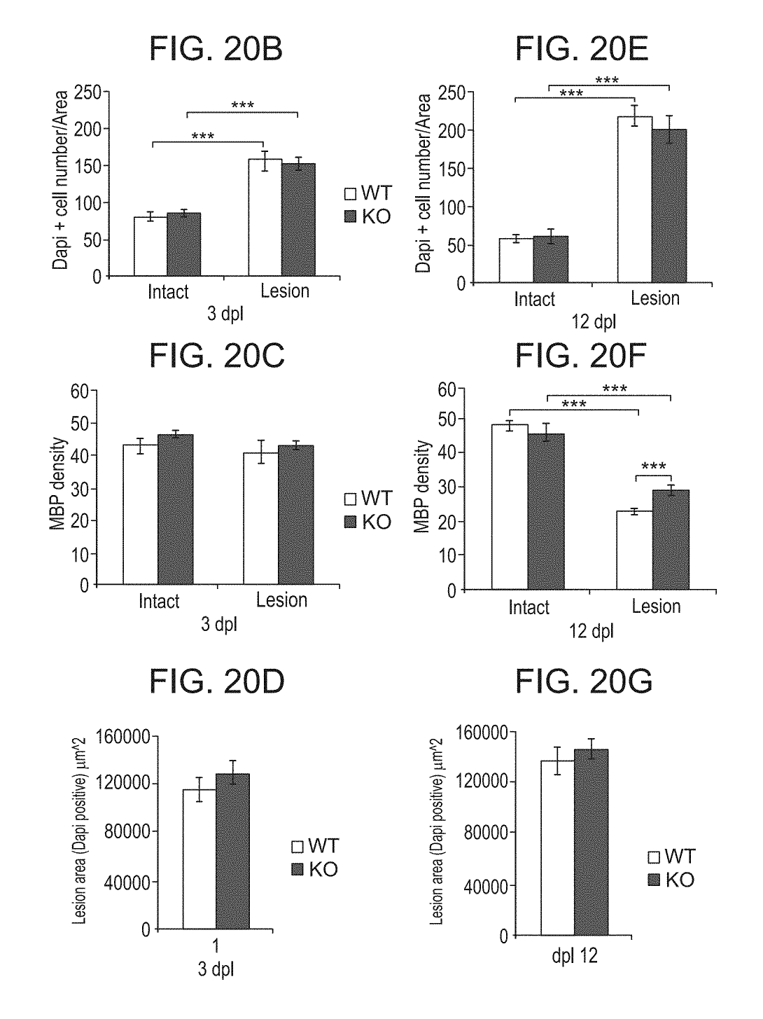

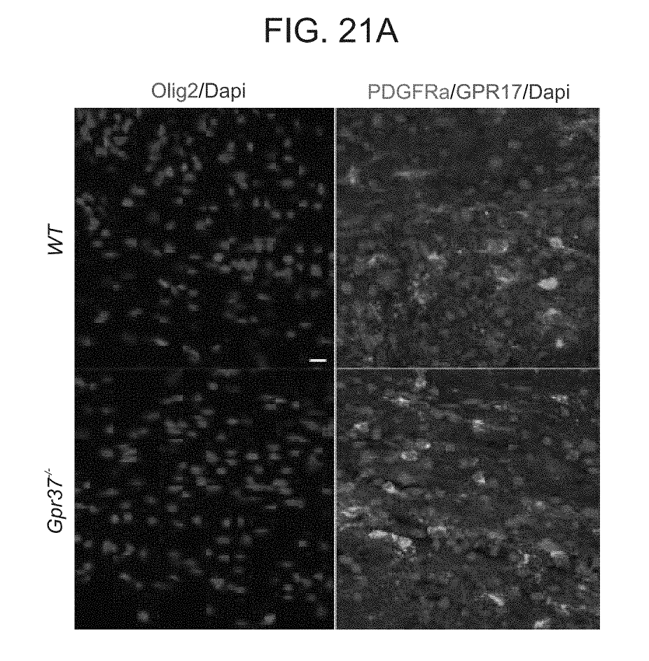

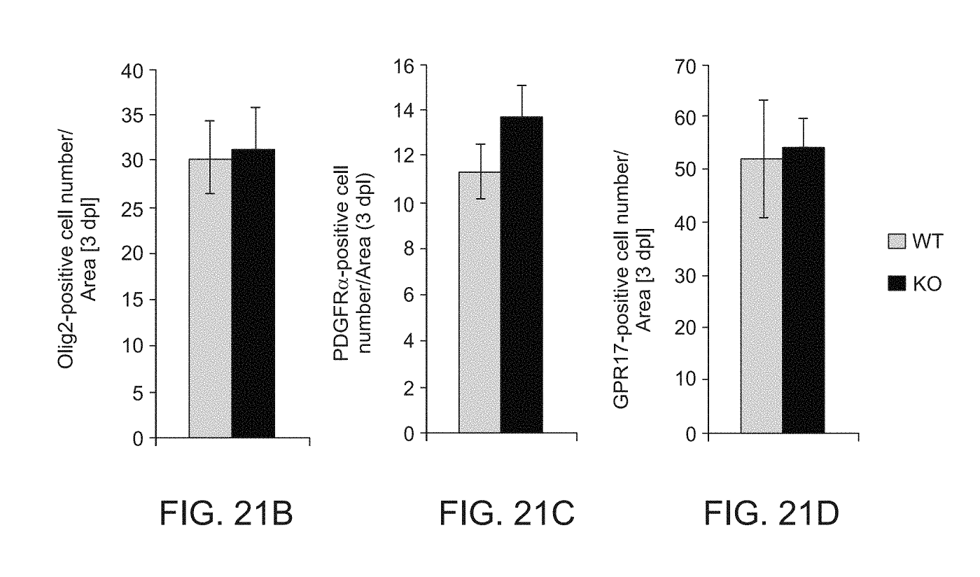

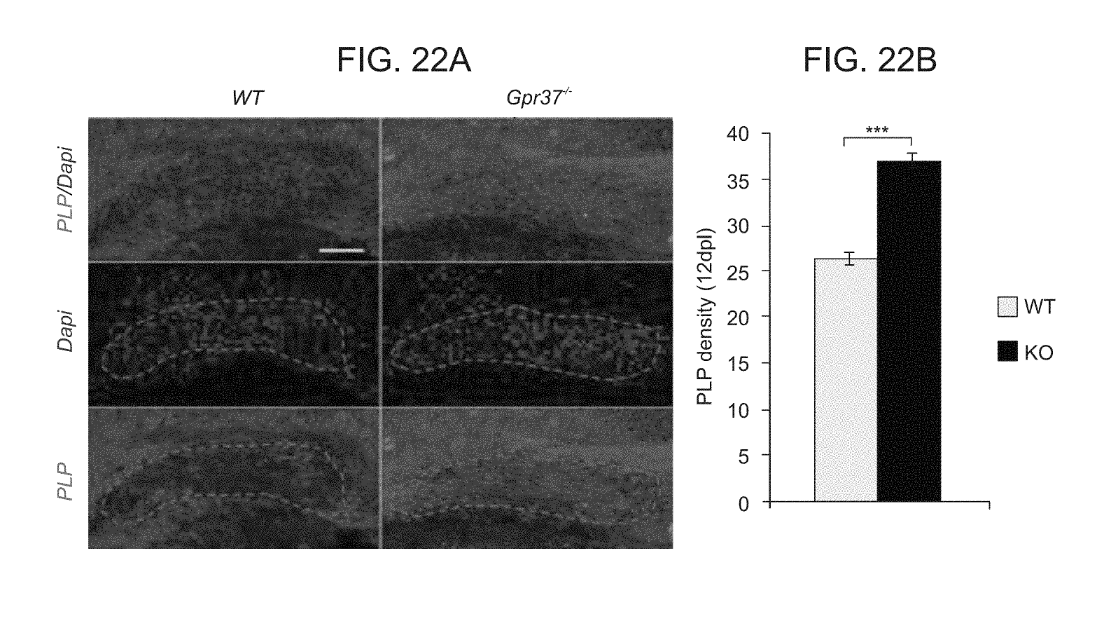

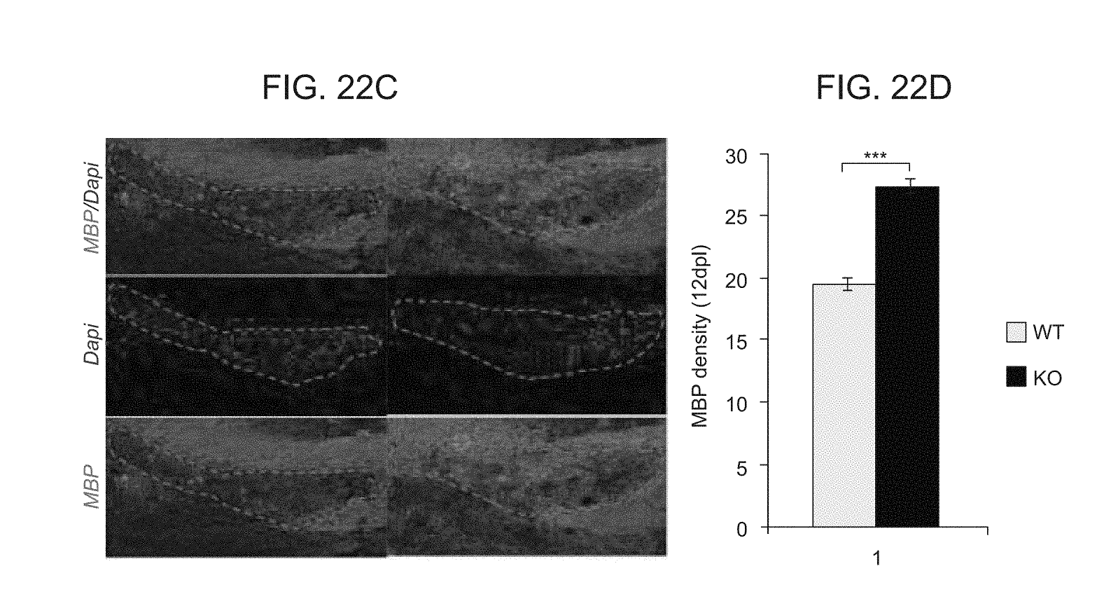

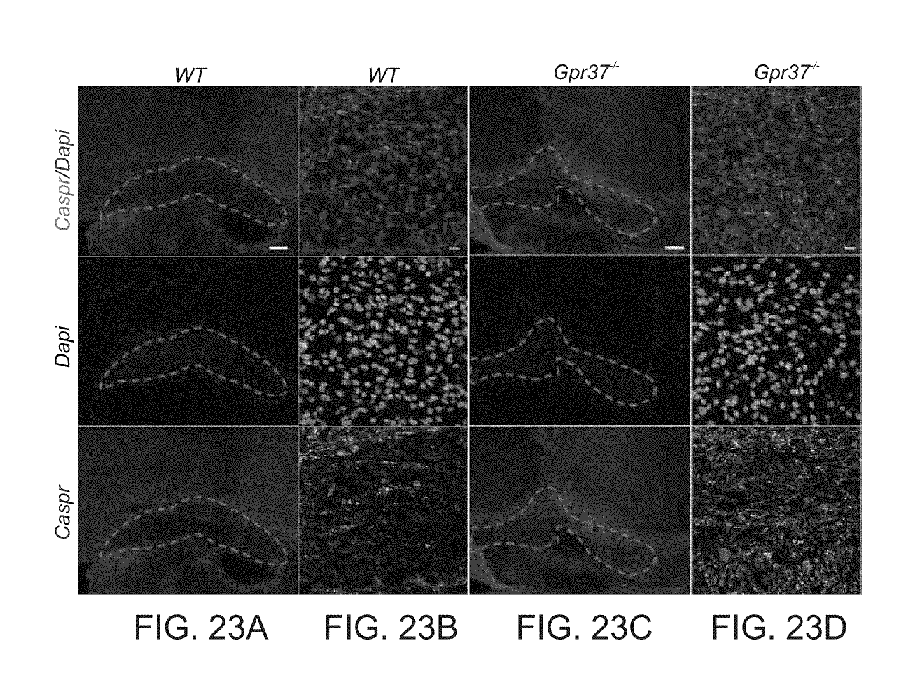

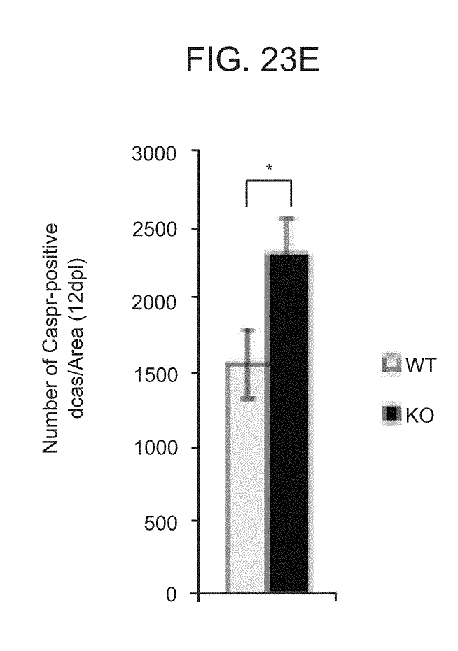

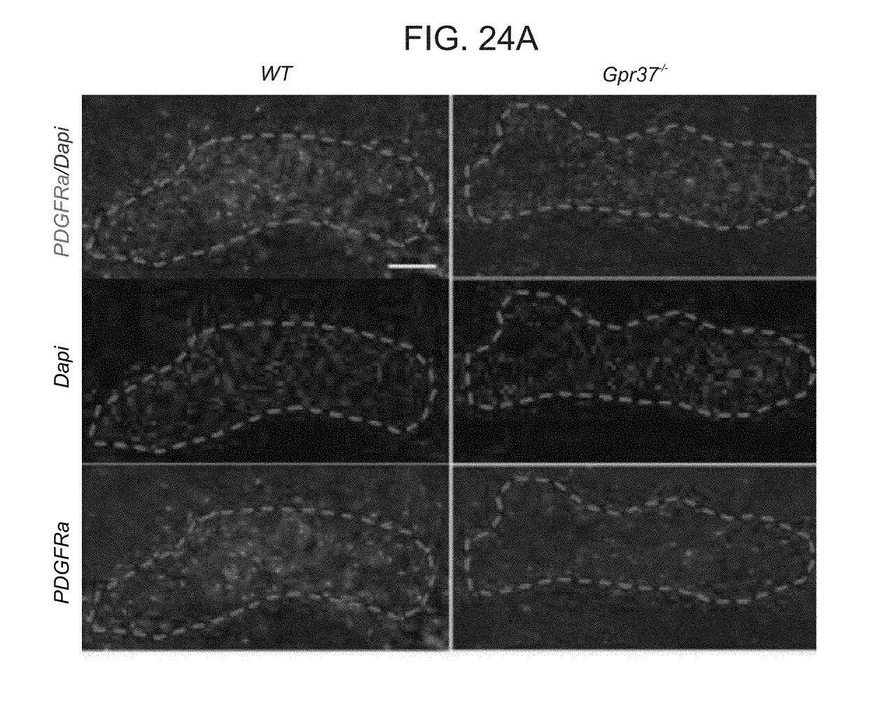

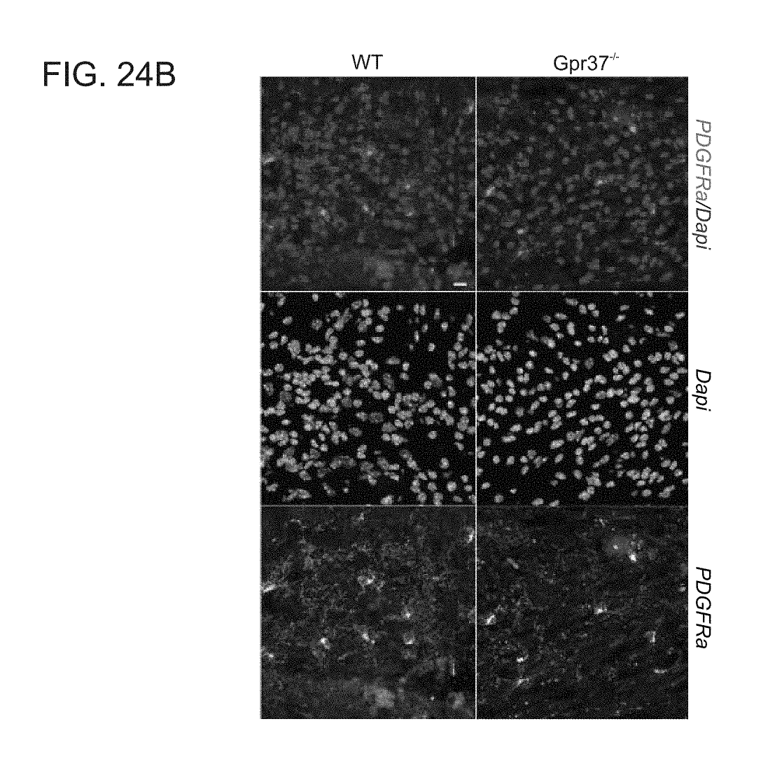

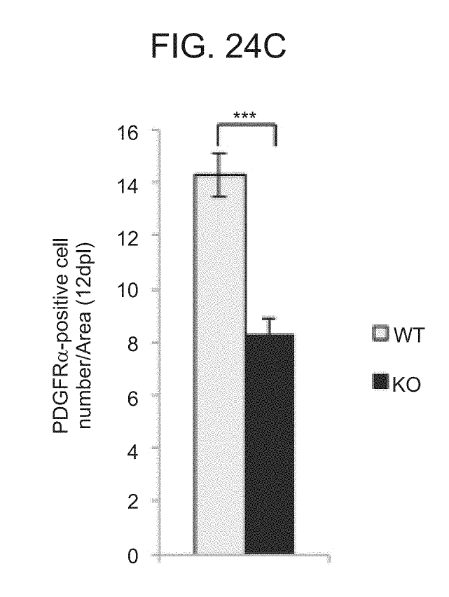

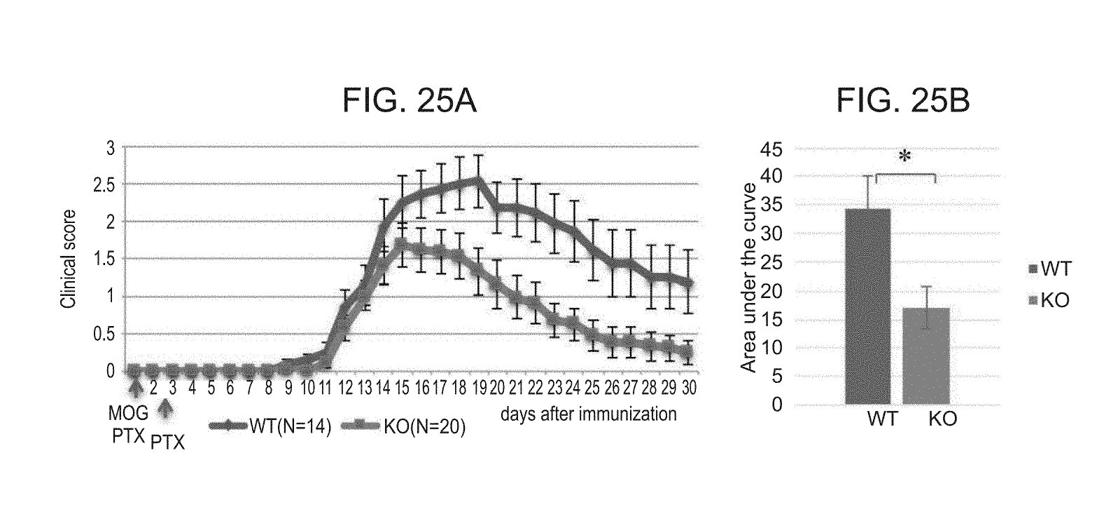

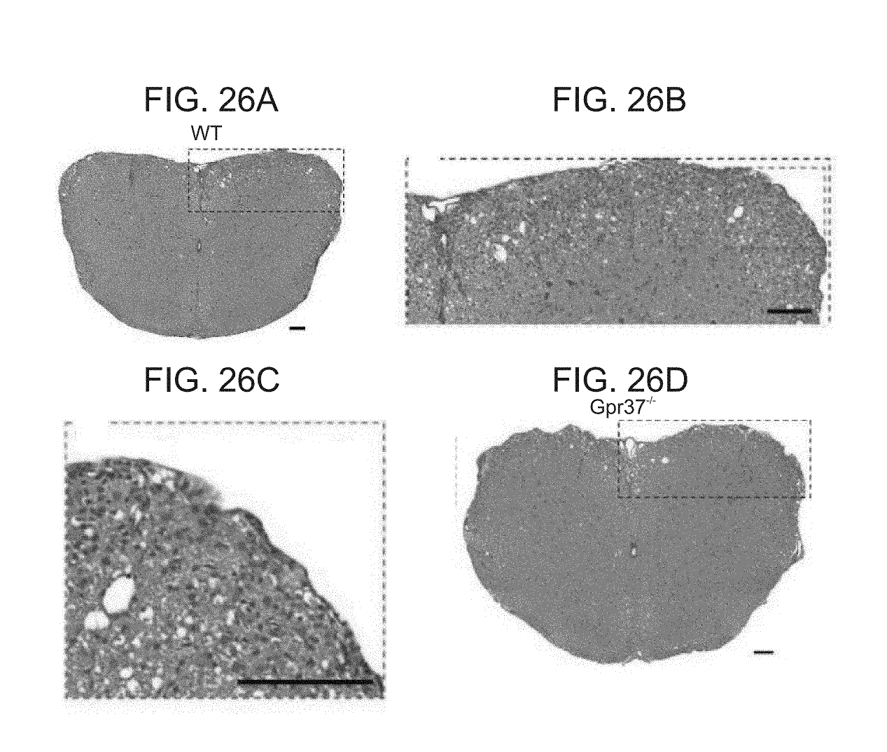

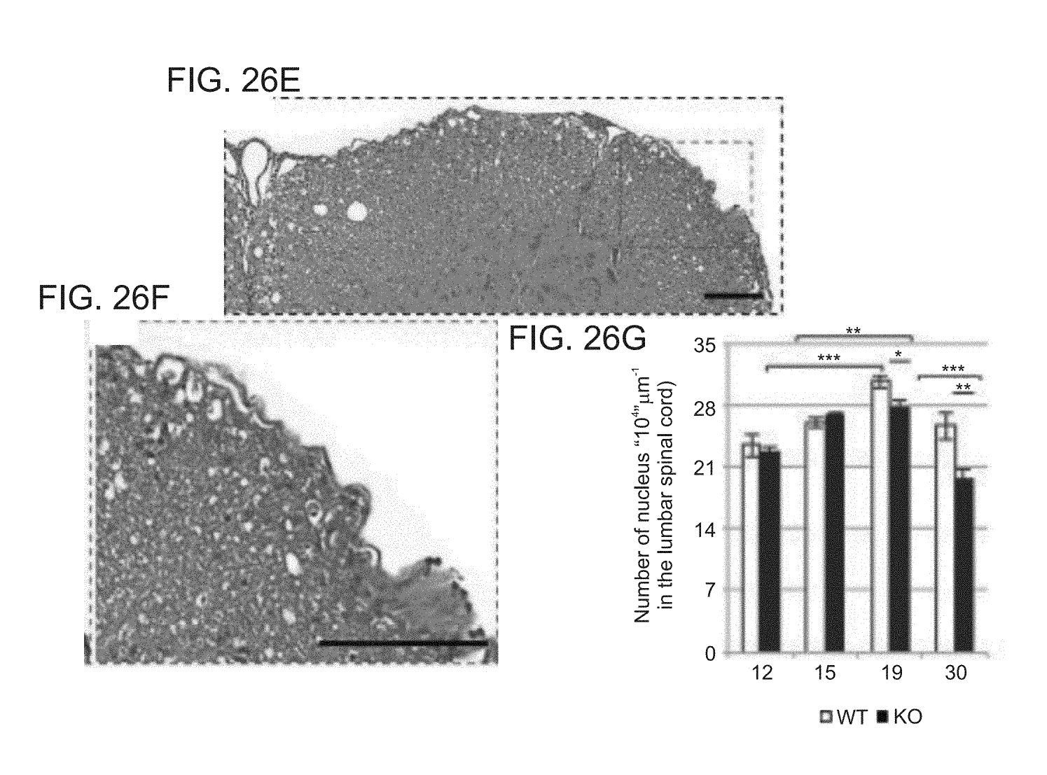

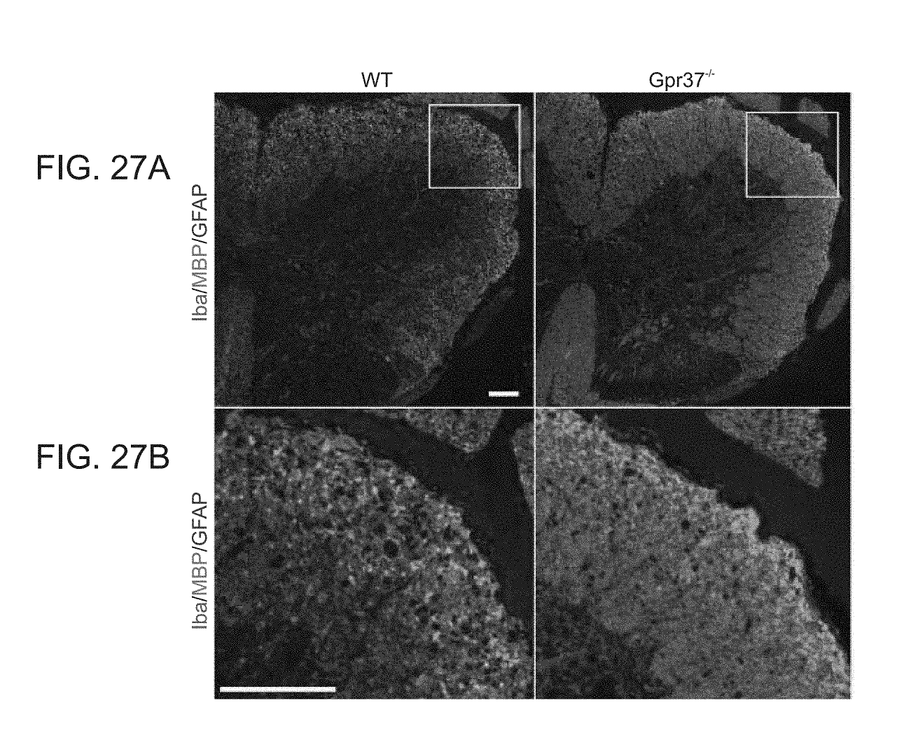

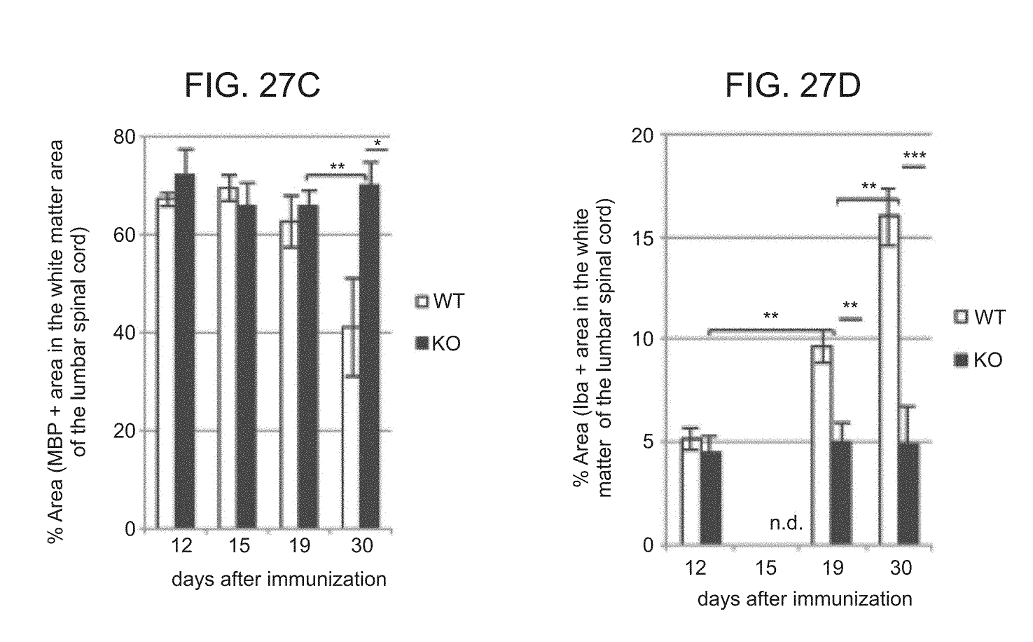

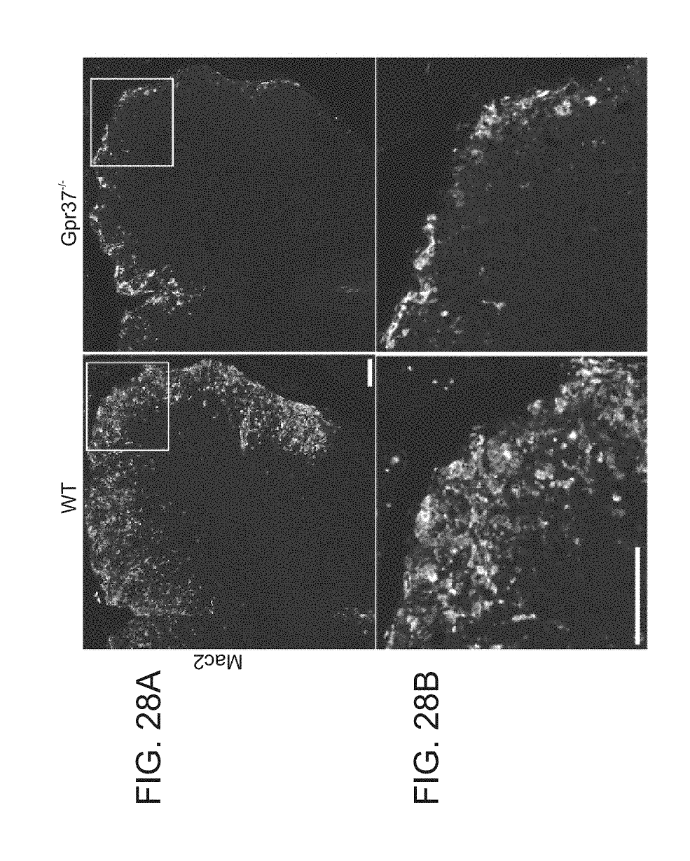

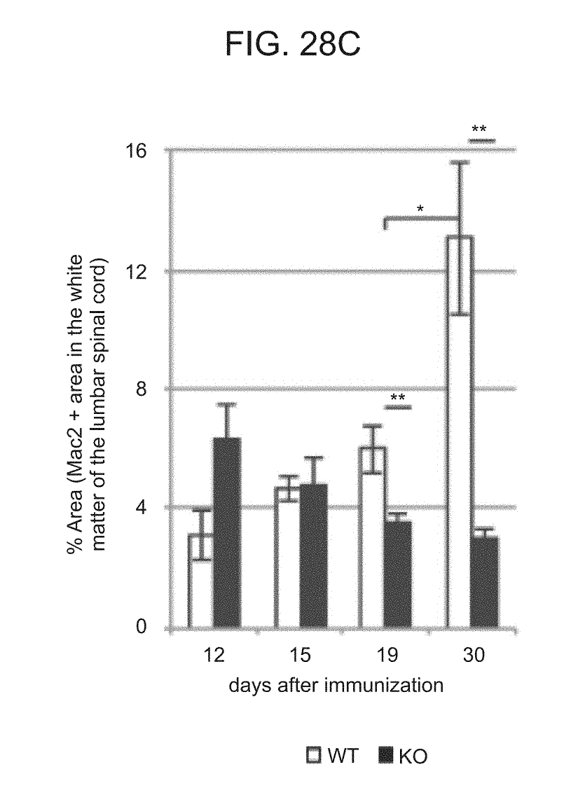

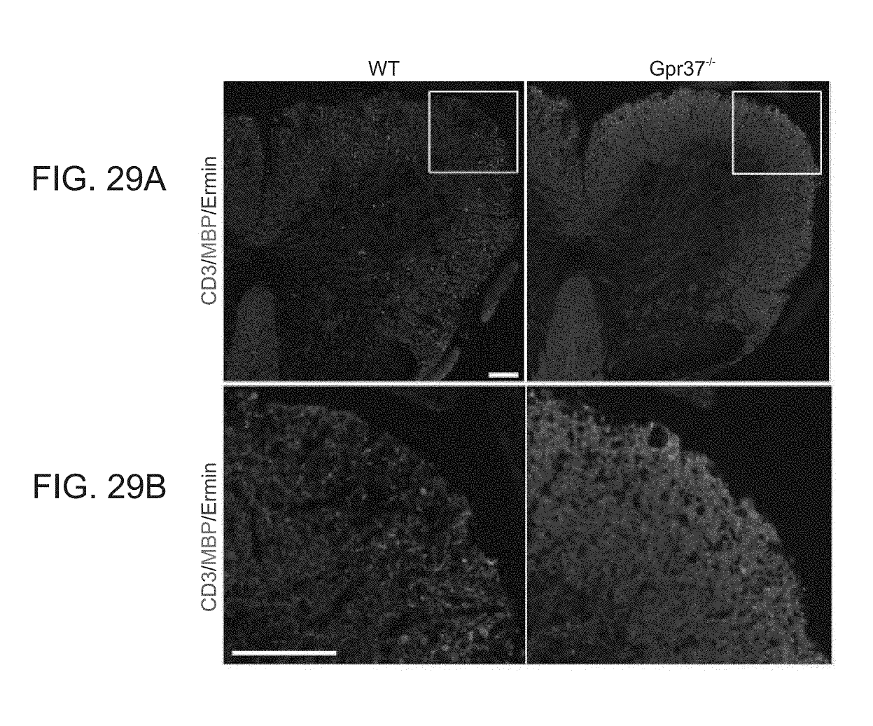

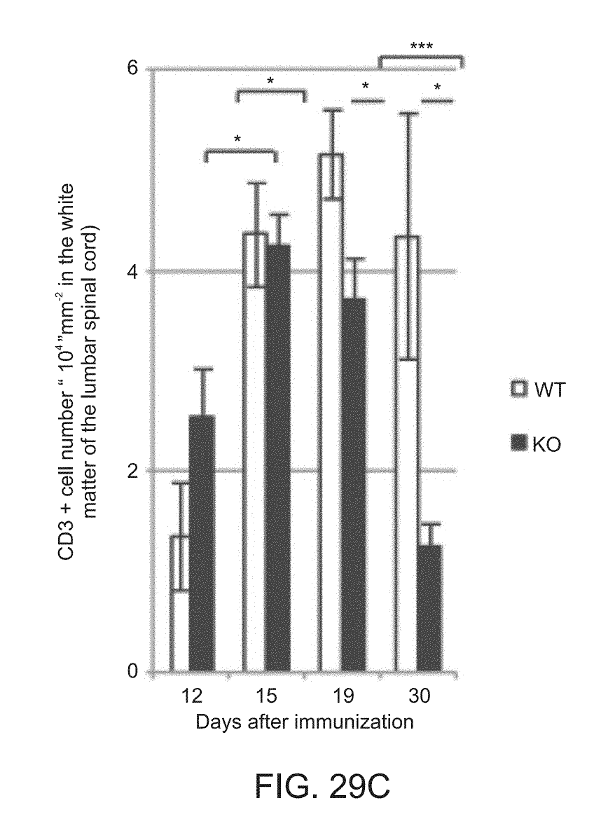

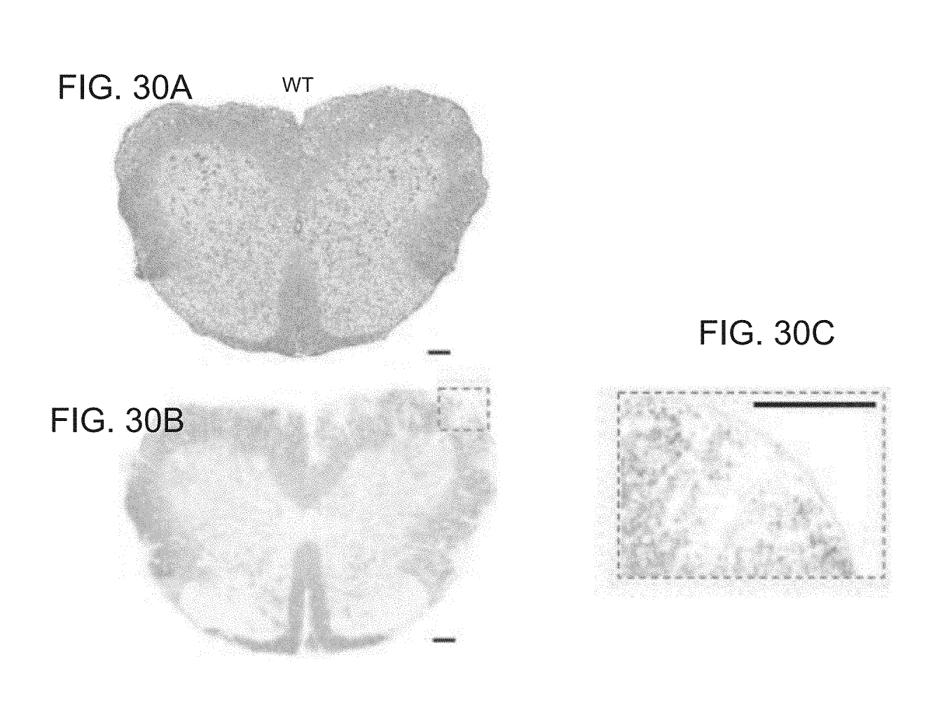

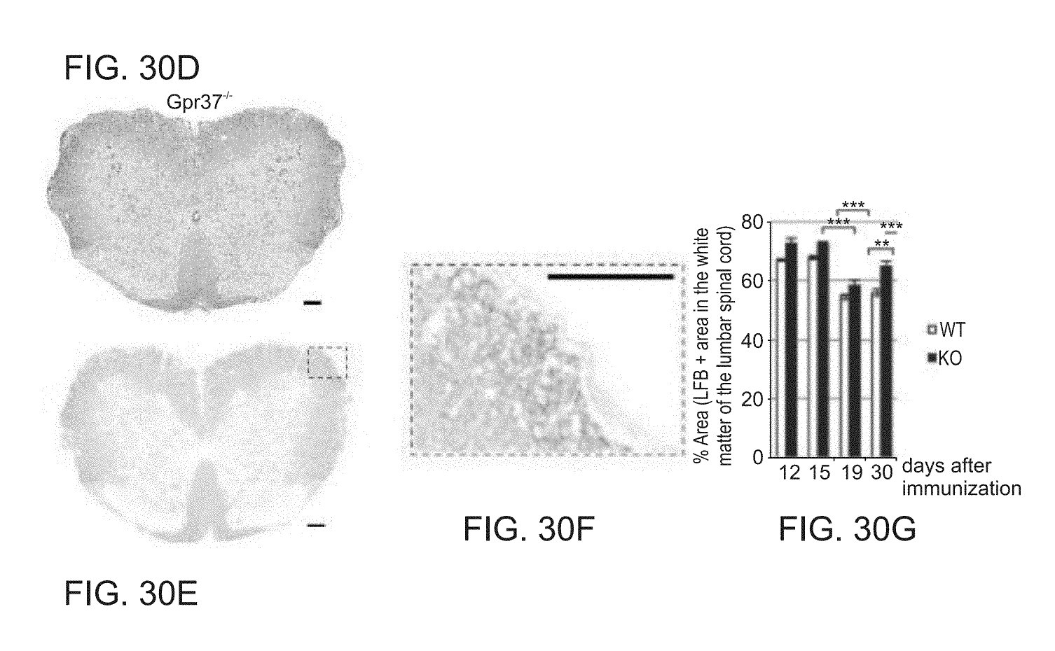

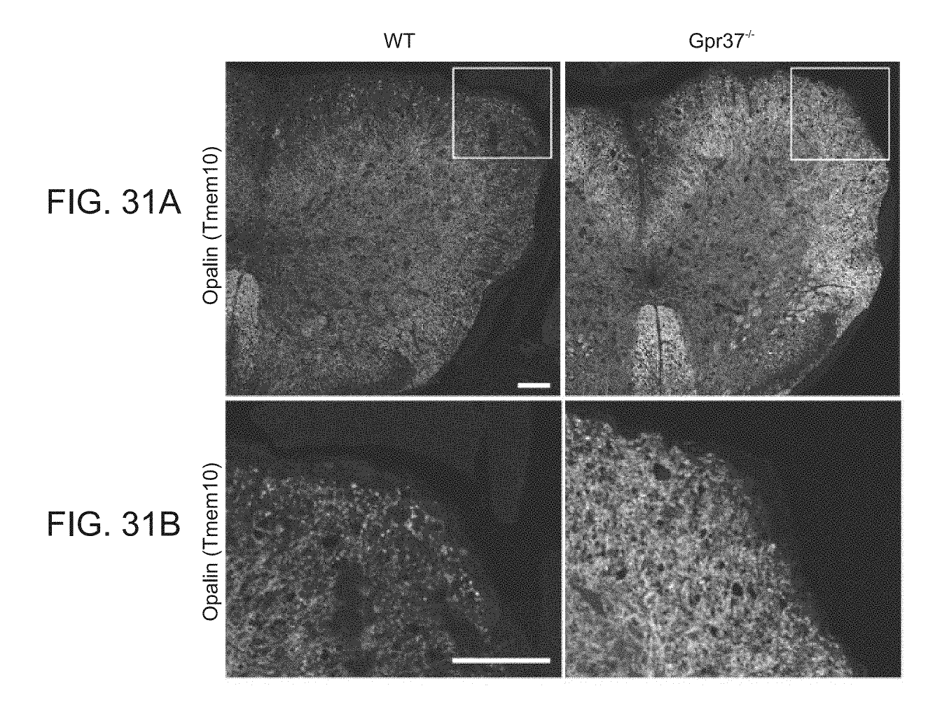

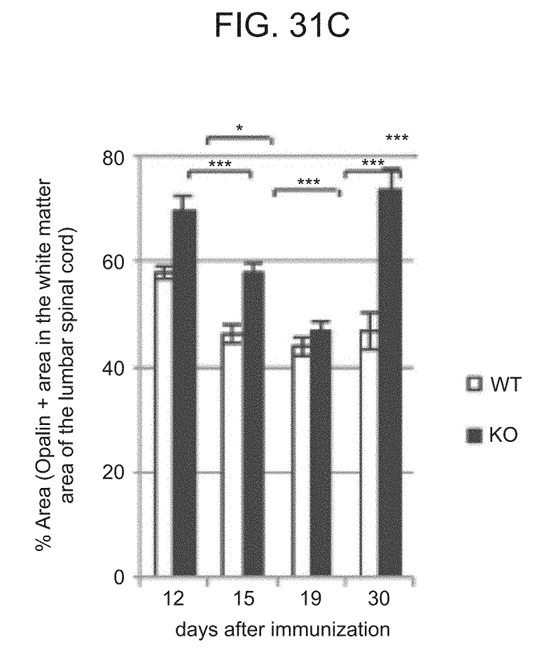

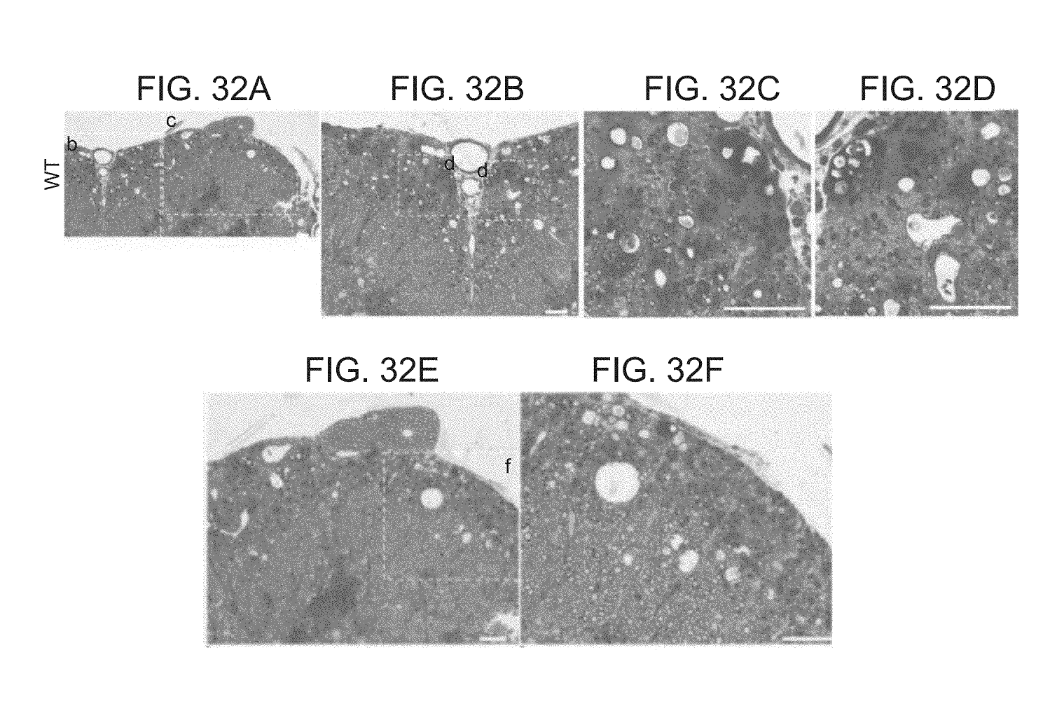

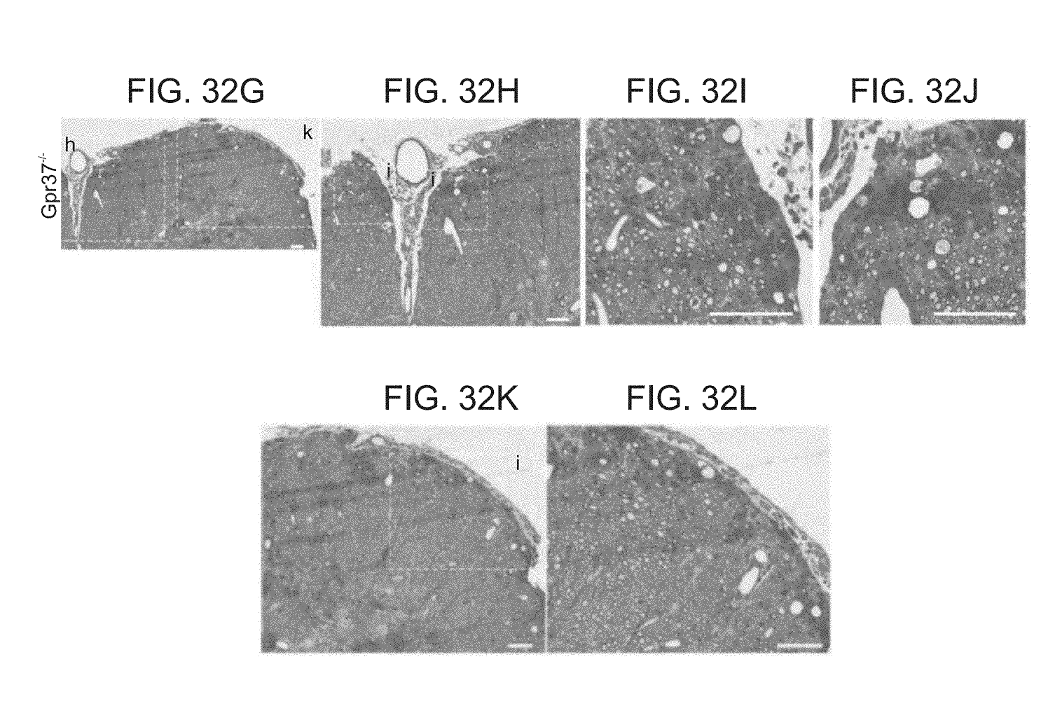

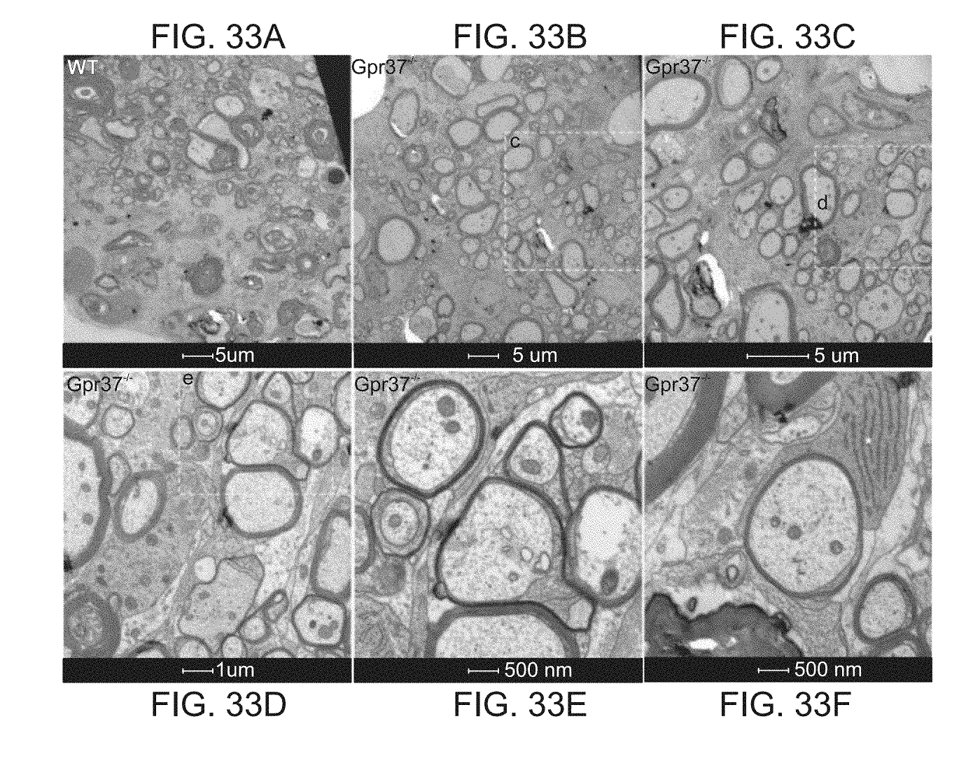

As is illustrated hereinunder and in the examples section, which follows, the present inventors show that GPR37 is highly enriched in oligodendrocytes compared to any other cell types in the CNS (FIGS. 1A-M). Furthermore, the present inventors show that expression of GPR37 begins relatively late during development after pre-myelinating oligodendrocytes have already formed (FIGS. 1N-Q). In corroboration, the present inventors found that OPC proliferation or early differentiation into O4 positive cells is not regulated by GPR37 (FIGS. 2A-J).

In addition, it has been shown that absence of GPR37 enhances the differentiation of pre-myelinating oligodendrocytes into myelin producing cells. Genetic deletion of Gpr37 in mice resulted in precocious differentiation of oligodendrocytes during development and a premature appearance of myelin in rostral areas of the brain (FIGS. 3A-B). Gpr37^{-/-} exhibits hypermyelination during development that last in the adult CNS (FIGS. 4A-I). As illustrated in FIGS. 5A-H, the inhibitory effect of GPR37 was shown to be mediated by suppression of ERK1/2, a signaling pathway known to control myelin thickness in the CNS. The observation that Gpr37^{-/-} oligodendrocytes cultured with wild type neurons exhibit precocious differentiation reveals that the function of GPR37 in oligodendrocytes is cell autonomous. This conclusion is also supported by the observation that shRNA knockdown of Gpr37 in oligodendrocytes results in their enhanced differentiation (FIGS. 8A-D).

Consequently, the present teachings suggest that agents which downregulate an amount and/or activity of Gpr37 would be efficacious in the treatment of diseases associated with aberrant myelination.

Thus, according to a first aspect of the present invention, there is provided a method of enhancing the myelinating activity of oligodendrocytes or progenitors thereof comprising contacting the oligodendrocytes or the progenitors with an agent that binds to G Protein-Coupled Receptor 37 (GPR37) or a polynucleotide encoding same or an upstream activator of the GPR37 so as to up-regulate an amount and/or activity of Extracellular Signal-Regulated Kinase 1/2 (ERK1/2) in the oligodendrocytes or the progenitors, thereby enhancing the myelinating activity of the oligodendrocytes or the progenitors.

As used herein, the term “myelin” refers to the fatty substance which encloses certain axons and nerve fibers, provides essential insulation, and enables the conductivity of nerve cells which transmit electrical messages to and from the brain.

The phrase “myelinating activity” refers to the ability of oligodendrocytes (i.e. processes extending therefrom) to wrap around neuronal axons and form myelin sheaths.

Oligodendrocyte precursor cells (OPCs) have the potential to differentiate into a myelinating phenotype (i.e. to increase myelination in-vitro), but are not capable, without further treatment (e.g. in the presence of an agent which induces further differentiation thereof), of myelinating in-vitro.

OPCs may also have the following functional phenotypes-a mitotic phenotype (i.e. they can divide and be expanded for three or more passages in culture) and migratory capacities.

The OPCs may comprise an elongated, bipolar or multipolar morphology. Typically, the OPCs can incorporate bromodeoxyuridine (BudR), a hallmark of mitosis.

Examples of OPC marker expression include, but are not limited to, PDGF-receptor, O4 sulfatide marker, Nkx2.2, Sox10, Olig1, Olig2, oligodendrocyte specific protein (OSP), 2′,3′-cyclic nucleotide-3′-phosphodiesterase (CNP), adenomatous polyposis coli (APC); NG2 (Chondroitin sulfate proteoglycan), A2B5, GD3 (ganglioside) and nestin.

According to a particular embodiment the OPCs of this aspect of the present invention express Olig1 and Olig2 to a greater extent (e.g. between 2-20 fold) than they express myelin basic protein (MBP) as measured in an identical assay. Such assays include analysis by Immunohistochemistry, RT-PCR and/or Western blot analysis.

In one embodiment the OPC expresses an early stage differentiation marker (e.g. Olig2). In another embodiment, the precursor expresses an intermediate state differentiation marker (O4). In still further embodiments, the precursor expresses a late stage differentiation marker (proteolipid protein (PLP)).

A myelinating oligodendrocyte may be identified by morphology and/or by the presence of a marker, e.g., myelin basic protein (MBP), myelin oligodendrocyte glycoprotein (MOG), 2′3′-cyclic-nucleotide 3′ phosphodiesterase (CNP), galactocebroside (GalC), O1 antigen (O1), or O4 antigen (O4), Protolopid protein (PLP), opalin and Ermin.

As mentioned, the method of this aspect of the present invention is carried out by contacting the above mentioned cells with agents that bind to GPR37 and down-regulate an amount of active GPR37. Since activation of GPR37 prevents an up-regulation in the amount/activity or ERK1/2, agents that down-regulate GPR37 should lead to an up-regulation in an amount or activity of ERK1/2 in oligodendrocytes or OPCs.

The agents of this aspect of the present invention may:

(i) bind to G Protein-Coupled Receptor 37 (GPR37) polypeptide;

(ii) bind to a polynucleotide encoding GPR37; and/or

(iii) bind to an upstream activator of GPR37 (e.g. prosaponsin and its derived peptides).

The polypeptide encoded by the GPR37 gene has the Uniprot No. 015354 and RefSeq No. NP_005293.1 (SEQ ID NO: 51).

The mRNA encoded by the GPR37 gene has the RefSeq No. NM_005302.3 (SEQ ID NO: 52).

ERK1 also known as Mitogen-Activated Protein Kinase 3 has the EC number EC 2.7.11.24 and Uniprot number MK03_HUMAN P27361. REFSEQ mRNAs are set forth in NM_001040056.2 (SEQ ID NO: 53), NM_001109891.1 (SEQ ID NO: 54) and NM_002746.2 (SEQ ID NO: 55)

ERK2 also known as Mitogen-Activated Protein Kinase 1 has the EC number EC 2.7.11.24 and Unitprot number MK01_HUMAN,P28482. REFSEQ mRNAs are set forth in NM_002745.4 (SEQ ID NO: 56) and NM_138957.3 (SEQ ID NO: 57).

In one embodiment, the amount of cellular ERK1/2 is increased in the oligodendrocytes or OPCs by the agents of this aspect of the present invention.

In another embodiment, the amount of nuclear ERK1/2 is increased in the oligodendrocytes or OPCs by the agents of this aspect of the present invention.

In still another embodiment, the activity of ERK1/2 is the ability to upregulate expression of myelin regulatory factor (Myrf). In still another embodiment, the activity of ERK1/2 is the ability to downregulate expression of HesS.

Downregulation of GPR37 can be effected on the genomic and/or the transcript level using a variety of molecules which interfere with transcription and/or translation [e.g., RNA silencing agents (e.g., antisense, siRNA, shRNA, micro-RNA), Ribozyme, DNAzyme and a CRISPR system (e.g. CRISPR/Cas)], or on the protein level using e.g., antagonists, enzymes that cleave the polypeptide and the like.

Following is a list of agents capable of binding to GPR37 or a polynucleotide encoding same and being capable of downregulating expression level and/or activity of GPR37.

One example, of an agent capable of downregulating GPR37 is an antibody or antibody fragment capable of specifically binding GPR37. Preferably, the antibody specifically binds at least one epitope of a GPR37. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2.

As used herein. the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Nati Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Downregulation of GPR37 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., GPR37) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A. et al. Brain Res. Protoc. 2004; 13:115-125; Diallo M., et al., Oligonucleotides. 2003; 13:381-392; Paddison P. J. et al., Proc. Natl Acad. Sci. USA. 2002; 99:1443-1448; Tran N., et al., FEBS Lett. 2004;573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, October 1, 2003, 13(5): 381-392. doi:10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 2lmers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

One exemplary shRNA molecule contemplated by the present inventors comprises the sequences SEQ ID NOs: 45 and 46. Another exemplary shRNA molecule contemplated by the present inventors comprises the sequences SEQ ID NOs: 47 and 48.

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (SEQ ID NO: 49; Brummelkamp, T. R. et al. (2002) Science 296: 550) and 5′-UUUGUGUAG-3′ (SEQ ID NO: 50; Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the GPR37 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of some embodiments of the invention need not be limited to those molecules containing only RNA, but further encompasses chemically-modified nucleotides and non-nucleotides.

According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 Genes Dev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above, that contacting oligodendrocytes or OPCs with a miRNA may be affected in a number of ways:
1. Transiently transfecting the oligodendrocytes or OPCs cells with the mature double stranded miRNA;
2. Stably, or transiently transfecting the oligodendrocytes or OPCs cells with an expression vector which encodes the mature miRNA.
3. Stably, or transiently transfecting the oligodendrocytes or OPCs cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.

14. Stably, or transiently transfecting the oligodendrocytes or OPCs with an expression vector which encodes the pri-miRNA The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

Another agent capable of downregulating a GPR37 is a DNAzyme molecule capable of specifically cleaving an mRNA transcript or DNA sequence of the GPR37. DNAzymes are single-stranded polynucleotides which are capable of cleaving both single and double stranded target sequences (Breaker, R.R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www(dot)asgt(dot)org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of GPR37 can also be effected by using an antisense polynucleotide capable of specifically hybridizing with an mRNA transcript encoding the GPR37.

Design of antisense molecules which can be used to efficiently downregulate GPR37 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

The current consensus is that recent developments in the field of antisense technology which have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating GPR37 is a ribozyme molecule capable of specifically cleaving an mRNA transcript encoding the GPR37. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Methods of introducing nucleic acid alterations to a gene encoding GPR37 are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Genome Editing using engineered endonucleases:—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo BiosciencesTM (Richmond, CA).

Method for designing and obtaining TALENs are described in e.g. Reyon et al. Nature Biotechnology 2012 May;30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through http://www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

An additional method of regulating the expression of GPR37 gene in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science,1989; 245:725-730; Moser, H. E., et al., Science,1987; 238:645-630; Beal, P. A., et al, Science,1992;251:1360-1363; Cooney, M., et al., Science,1988;241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the GPR37 regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999;27:1176-81, and Puri, et al, J Biol Chem, 2001; 276:28991-98), and the sequence-and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al., J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003;112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al., and U.S. Pat. No. 5,721,138 to Lawn.

Another agent capable of downregulating GPR37 would be an agent which binds to GPR37 and blocks the agonist from binding thereto. Alternatively, the agent may be capable of cleaving GPR37. Such molecules can be GPR37 antagonists, or GPR37 inhibitory peptides.

Another agent which can be used along with some embodiments of the invention to downregulate GPR37 is a molecule which binds to and sequesters the natural agonist of GPR37.

In one embodiment, the oligodendrocytes and/or OPCs are contacted in vitro or ex vivo with any one of the above described agents.

The oligodendrocytes and/or OPCs may be derived from a subject (e.g. a primary culture, a cell line). The subject from whom the oligodendrocytes are derived may be healthy or non-healthy, e.g. suffering from a myelinating disorder. Other exemplary oligodendrocyte cells contemplated by the present invention include those disclosed in PCT publication WO 97/32608 and U.S. Pat. No. 5,830,621.

In another embodiment, the oligodendrocytes and/or OPCs may be differentiated ex-vivo from stem cells such as embryonic stem cells, mesenchymal stem cells or induced pluripotent stem cells.

As used herein, the phrase “stem cells” refers to cells which are capable of remaining in an undifferentiated state (e.g., pluripotent or multipotent stem cells) for extended periods of time in culture until induced to differentiate into other cell types having a particular, specialized function (e.g., fully differentiated cells). Preferably, the phrase “stem cells” encompasses embryonic stem cells (ESCs), induced pluripotent stem cells (iPS), adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Placental and cord blood stem cells may also be referred to as “young stem cells”.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ES cells can be purchased from the NIH human embryonic stem cells registry [.www.grants (dot)nih(dot)gov/stem_cells/registry/current(dot)html]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUESS, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBRS, WIBR6, HUES 45, Shef 3, Shef 6, BJNhem19, BJNhem20, SA001, SA001.

In addition, ES cells can be obtained from other species as well, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci U S A. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 February 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T. et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M. et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis.

Adult tissue stem cells can be isolated using various methods known in the art such as those disclosed by Alison, M. R. [J Pathol. 2003 200(5): 547-50], Cal, J. et al., [Blood Cells Mol Dis. 2003 31(1): 18-27], Collins, A. T. et al., [J Cell Sci. 2001; 114(Pt 21): 3865-72], Potten, C. S. and Morris, R. J. [Epithelial stem cells in vivo. 1988. J. Cell Sci. Suppl. 10, 45-62], Dominici, M et al., [J. Biol. Regul. Homeost. Agents. 2001, 15: 28-37], Caplan and Haynesworth [U.S. Pat. No. 5,486,359] Jones E. A. et al., [Arthritis Rheum. 2002, 46(12): 3349-60]. Fetal stem cells can be isolated using various methods known in the art such as those disclosed by Eventov-Friedman S, et al., PLoS Med. 2006, 3: e215; Eventov-Friedman S. et al., Proc Natl Acad Sci U S A. 2005, 102: 2928-33; Dekel B. et al., 2003, Nat Med. 9: 53-60; and Dekel B. et al., 2002, J. Am. Soc. Nephrol. 13: 977-90. Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton.

Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62]. Thus, an adult tissue such as, for example, prostate tissue is digested with Collagenase and subjected to repeated unit gravity centrifugation to separate the epithelial structures of the prostate (e.g., organoids, acini and ducts) from the stromal cells. Organoids are then disaggregated into single cell suspensions by incubation with Trypsin/EDTA (Life Technologies, Paisley, UK) and the basal, CD44-positive, stem cells are isolated from the luminal, CD57-positive, terminally differentiated secretory cells, using anti-human CD44 antibody (clone G44-26; Pharmingen, Becton Dickinson, Oxford, UK) labeling and incubation with MACS (Miltenyi Biotec Ltd, Surrey, UK) goat anti-mouse IgG microbeads. The cell suspension is then applied to a MACS column and the basal cells are eluted and re-suspended in WAJC 404 complete medium [Robinson, E. J. et al. (1998). Basal cells are progenitors of luminal cells in primary cultures of differentiating human prostatic epithelium Prostate 37, 149-160].

In one embodiment, the stem cells utilized by some embodiments of the invention are BM-derived stem cells including hematopoietic, stromal or mesenchymal stem cells (Dominici, M et al., 2001. Bone marrow mesenchymal cells: biological properties and clinical applications. J. Biol. Regul. Homeost. Agents. 15: 28-37). BM-derived stem cells may be obtained from iliac crest, femora, tibiae, spine, rib or other medullar spaces.

Methods of differentiating stem cells ex vivo towards an oligodendrocyte lineage are known in the art and are disclosed for example in US Patent Application No. 20100003751, US Patent Application No. 20100021434, U.S. Pat. No. 5,968,829 and US Patent Application No. 20120009673, the contents of all are incorporated herein by reference.

It will be appreciated that since the present inventors have shown that down-regulation of GPR37 affects the final stages of oligodendrocyte differentiation, preferably the agent which targets GPR37 is added during the terminal stages of differentiation.

The conditions used for incubating the cells are selected for a time period/concentration of cells/concentration of agent/ratio between cells and agent and the like which enable the agent to induce cellular changes, such as changes in transcription and/or translation rate of specific genes, proliferation rate, differentiation, cell death, necrosis, apoptosis and the like.

In order to test the myelinating activity of the cells generated after incubating with an agent described herein above myelin protein expression can be assayed myelin protein expression e.g. by immunocytochemistry and Western blot. Alternatively, the myelinating activity of the agent can be tested by co-culturing the cells with neuronal cells (e.g. dorsal root ganglion (DRG) neuronal axons) and the number of co-localized MBP positive fibers and/or Neurofilament positive fibers can be compared to non-treated cells.

Additional examples of assays that can be used to assess activity are described in Najm et al. Nat Methods. 2011 Sep. 25; 8(11):957-62; Bai et al. Neurosci Bull. 2013 April; 29(2):239-50; Yang et al. Dev Biol. 2011 Feb. 1; 350(1):127-38; and Cho et al. Curr Neuropharmacol. 2007 March; 5(1): 19-33.

In some embodiments, the agents can be further tested using a brain slice ex vivo assay that assesses myelination the brains of mammals, (e.g., rats and mice). Such assays are described, for example, in Bai et al. Neurosci Bull. 2013 April; 29(2):239-50, Yang et al. Dev Biol. 2011 Feb. 1; 350(1):127-38, and Cho et al. Curr Neuropharmacol. 2007 March; 5(1): 19-33.

In some embodiments, the compounds can be further screened using an in vivo assay that assesses remyelination and reduction of clinical severity in the MOG₃₅₋₅₅-induced chronic experimental autoimmune encephalomyelitis (EAE) rodent model of multiple sclerosis.

It will be appreciated that as well as contacting oligodendrocytes or OPCs with the agents in vitro or ex vivo, the present inventors also contemplate contacting cells in vivo with the agents. Typically, the agents are administered to a subject in order to treat a disease.

Thus, according to another aspect of the present invention there is provided method of treating a demyelinating disease of a subject in need thereof, comprising administering to the subject a therapeutically effective amount of an agent that binds to GPR37 or a polynucleotide encoding same or an upstream activator of said GPR37 so as to up-regulate an amount and/or activity of ERK1/2 in oligodendrocytes or progenitors of the subject, thereby treating the demyelinating disease.

The subject of this aspect of the present invention is typically a mammalian subject, e.g. a human.

As used herein, the term “demyelinating disease” refers to a disease or condition of the nervous system characterized by damage to or loss of the myelin sheath of neurons. A demyelinating disease can be a disease affecting the central nervous system or a disease affecting the peripheral nervous system. Examples of demyelinating diseases include, but are not limited to multiple sclerosis, optic neuritis, an idiopathic inflammatory demyelinating disease, Guillain-Barre Syndrome, chronic inflammatory demyelinating polyneuropathy, transverse myelitis, Balo concentric sclerosis, pernicious anemia, central pontine myelinolysis, Tabes dorsalis, neuromyelitis optica (NMO), progressive multifocal leukoencephalopathy (PML), anti-MAG (myelin-associated glycoprotein) neuropathy, hereditary motor and sensory neuropathy (Chacot-Marie-Tooth disease), cerebrotendinious xanthanomatosis, and leukodystrophies including adrenoleukodystrophy, adrenomyeloneuropathy, metachromatic leukodystrophy, globoid cell leukodystrophy (Krabbe disease), Canavan disease, vanishing white matter disease, Alexander disease, Refsum disease, and Pelizaeus-Merzbacher disease.

According to a particular embodiment, the disease is multiple sclerosis (MS).

Additional diseases that may be treated according to this aspect of the present invention include age-related white matter involution of subcortical dementia (white matter dementia) and periventricular leukomalacia of cerebral palsy.

Other diseases where remyelination and/or myelin loss prevention would be beneficiary include white matter stroke and spinal cord injury. Furthermore diseases where oligodendrocytes are affected such as frontotemporal dementias, schizophrenia and degenerative disorders (e.g. normal aging and Alzheimer's disease) are also contemplated for treating with the agents of the present invention. Additional diseases include those disclosed in Filley et al., Ther Adv Neurol Disord (2012) 5(5) 267-277 and Goldman et al.,Science, Vol. 338 26 Oct. 2012.

It will be appreciated that as well as providing the agent directly to the subject, the present inventors also contemplate treating cells ex vivo with the agent and subsequently administering the cells to the subject.

Thus, according to still another aspect of the present invention there is provided a method of treating a demyelinating disease of a subject in need thereof, comprising:

(a) contacting oligodendrocytes or progenitors thereof with an agent that binds to GPR37 or a polynucleotide encoding same or an upstream activator of said GPR37 so as to generate cells with an enhanced myelinating activity; and

(b) providing said cells with an enhanced myelinating activity to said subject, thereby treating the demyelinating disease.

The cells of this aspect of the present invention may be derived originally from the subject who is being treated or may be derived from another source.

It will be appreciated that the agents which down-regulate GPR37 may be provided in conjunction with additional agents that inhibit one of the down-stream effectors of GPR37. Thus, for example, the present invention contemplates providing agents that modulate the amount of cAMP, the activation of Raf, MEK, ERK1/2, or increase translocation of ERK1/2 into the nucleus.

It is proposed that when a subject is being treated with an agent that causes an inhibition in the amount/activity of GPR37, care should be taken so as not to expose the subject to demyelinating toxins.

The agents or cells of the present invention may be provided to the subject per se or may be administered as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent, or cells which were treated with the agent, accountable for the biological effect (i.e. those that modulate the amount and/or activity of GPR37).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., demyelinating disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays.

For example, a dose can be formulated in animal models (e.g. EAE model for multiple sclerosis) to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to ensure that levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Since the present inventors have shown that the polypeptide GPR37 is involved in myelination, it is proposed that cells that express GPR37 may be used to screen for agents useful for treating demyelinating diseases.

Thus, according to still another aspect of the present invention there is provided a method of selecting an agent which is capable of increasing the myelinating activity of an oligodendrocyte comprising:

(a) contacting cells which express GPR37 with a candidate agent; and

(b) measuring an amount and/or activity of said GPR37, wherein when said amount and/or activity is below a predetermined level, it is indicative that the candidate agent is capable of increasing the myelinating activity of an oligodendrocyte.

Exemplary cells that may be used in the assay include oligodendrocytes or progenitors thereof. Additionally, or alternatively, the cells may be genetically modified so as to express GPR37.

An exemplary cell line contemplated by the present inventors which expresses GPR37 is produced by DiscoverX, catalogue number 93-0339C2A.

Additional information regarding screening assays may be found in Bohn et al, Drug Discov Today Technol. 2010; 7(1): e37-e42. doi:10.1016/j.ddtec.2010.06.005, the contents of which is incorporated herein by reference.

Once the agent has been qualified as being able to reduce GPR37 it may be tested using additional assays such as its ability to enhance myelination of neurons in vitro or to induce and promote differentiation and/or maturation of oligodendrocyte precursor cells. Additionally, the agent may be tested on animal models of MS.

Furthermore, the agent may be tested for potency and toxicity.

In one embodiment, the assay of this aspect of the present invention may be used to screen small molecule agents.

The term “small molecule” as used herein refers to biologically active organic compounds of low molecular weight (e.g. <1000 or <500 kDa).

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### G Protein Coupled Receptor 37 is a Negative Regulator of Oligodendrocyte Differentiation and Myelination

Materials and Methods

Mice: Gpr37^{+/-} (B6.129P2-Gpr37^{tmlDgen}/J) mice were obtained from the Jackson Laboratory and were maintained on a C57BL/6J background. Age of mice used for experiments is detailed in the figure legends; male and female littermate mice were used interchangeably. Mice were genotyped for the targeted allele by PCR using tail DNA. For the targeted allele, the forward primer in neo, 5′-gggtgggattagataaatgcctgctct-3′ (SEQ ID NO: 1) and the gene-specific reverse primer, 5′-ggccaagagagaattggagatgctc-3′ (SEQ ID NO: 2) were used. For the endogenous allele, the gene-specific forward primer, 5′-aacgggtctgcagatgactgggttc-3′ (SEQ ID NO: 3), and the gene-specific reverse primer, 5′-ggccaagagagaattggagatgctc-3′ (SEQ ID NO: 4), were used. All experiments were performed in compliance with the relevant laws and institutional guidelines, and were approved by the Weizmann Institute's Animal Care and Use Committee.

RT-PCR and qRT-PCR. Total RNA was isolated with TRIzol reagent (Sigma) from freshly dissected mouse brains or cultured cells. Isolated RNA was treated with DNasel to eliminate genomic DNA before reverse transcription. Mouse cDNAs were prepared using SuperScript II Reverse Transcriptase (Invitrogen). Specific primer sets were used for GPR37 (forward, CCTGCAAGATCGTGCCCTA (SEQ ID NO: 5); reverse, AGTACATCTGGACGTTGGTGG (SEQ ID NO: 6)), GPR37L1 (forward, CTTTAGGTGGGCATAGAGC (SEQ ID NO: 7); reverse, TGGAGAACTGGTTGATGAGGC (SEQ ID NO: 8)), MAG (forward, TGCCGCTGTTTTGGATAA (SEQ ID NO: 9); reverse, CGCCTCGGAAATAGTATTTG (SEQ ID NO: 10)), MBP (forward, CCAGAGCGGCTGTCTCTTCC (SEQ ID NO: 11); reverse, CATCCTTGACTCCATCGGGCGC (SEQ ID NO: 12)) and actin (forward, GAGCACCCTGTGCTGCTCACCGAGG (SEQ ID NO: 13); reverse, GTGGTGGTGAAGCTGTAGCCACGCT (SEQ ID NO: 14)). Quantitative RT-PCR was performed in the PCR 7900HT Real-Time PCR System (Applied Biosystems) using SYBR Green mix. The following primers were used: GPR37 (forward, TGTCACTACCTTCACCTTATG (SEQ ID NO: 15); reverse, GCTCCAACCCAGATAACG (SEQ ID NO: 16)), GPR17 (forward CAGCTACGAGGAGTCCACCTGGAGCAC (SEQ ID NO: 17); reverse, CGGTAGGGCTGCCTCCAGACCGTTCAT (SEQ ID NO: 18)), Hes5 (forward, AACTCCAAGCTGGAGAAGGC (SEQ ID NO: 19); reverse, GTCAGGAACTGTACCGCCTC (SEQ ID NO: 20)), Hes1 (forward, AATGACTGTGAAGCACCTCC (SEQ ID NO: 21); reverse, ATTTCCCCAACACGCTCGG (SEQ ID NO: 22)), Id2 (forward, CCTGCATCACCAGAGACCTG (SEQ ID NO: 23); reverse, TTCGACATAAGCTCAGAAGGGAA (SEQ ID NO: 24)), Tcf4 (forward, CATCACCAACAGCGAATGGC (SEQ ID NO: 25); reverse, CACTGCTTACAGGAGGCGAA (SEQ ID NO: 26)), Ascll (forward, CAACCGGGTCAAGTTGGTCA (SEQ ID NO: 27); reverse, CTCATCTTCTTGTTGGCCGC (SEQ ID NO: 28)), Oligl (forward, GCTCGCCCAGGTGTTTTGT (SEQ ID NO: 29); reverse, GCATGGAACGTGGTTGGAAT (SEQ ID NO: 30)), Olig2 (forward, AGAGCCAGGTTCTCCTCCG (SEQ ID NO: 31); reverse, ACTAGACACCAGGCTGGCGT (SEQ ID NO: 32)), Nkx2.2 (forward, GGTTCCAGAACCATCGCTACA (SEQ ID NO: 33); reverse, GCTTCGATCCTGGCATCCAT (SEQ ID NO: 34)), Nkx6.2 (forward, CATGACCGAGAGCCAAGTGA (SEQ ID NO: 35); reverse, GCTTCTTTTTAGCCGACGCC (SEQ ID NO: 36)), Sox10 (forward, AGCCCAGGTGAAGACAGAGA (SEQ ID NO: 37); reverse, AGTCAAACTGGGGTCGTGAG (SEQ ID NO: 38)), Yy1 (forward, TTGAGCTCTCAACGAACGCTTTGC (SEQ ID NO: 39); reverse, TCAGACCCTAAGCAACTGGCAGAA (SEQ ID NO: 40)), and Myrf (forward, TGGCAACTTCACCTACCACA (SEQ ID NO: 41); reverse, GTGGAACCTCTGCAAAAAGC (SEQ ID NO: 42)). GAPDH (forward, GGTCGGTGTGAACGGATTTG (SEQ ID NO: 43); reverse, TCGTTGATGGCAACAATCTCCACT (SEQ ID NO: 44)) was used as reference genes. All reactions were carried out in triplicate and GAPDH was used for normalization.

shRNA. Oligonucleotides used for the generation of the pSUPER retroviral vectors were as follows:

GPR37-sh1 sense:
(SEQ ID NO: 45)
gatctccccaacgtccagatgtactattcaagagatagtacatctggacg
ttggtattc;
GPR37-sh1 antisense:
(SEQ ID NO: 46)
tcgagaaaaaccaacgtccagatgtactatctcttgaatagtacatctgg
acgttgggga;
GPR37-sh2 sense:
(SEQ ID NO: 47)
gatctccgaaggccagtacccgtggattcaagagatccacgggtactggc
cttctttttc;
GPR37-sh2 antisense:
(SEQ ID NO: 48)
cgagaaaaagaaggccagtacccgtggatctcttgaatccacgggtactg
gccttcgga.

Retroviruses were Produced by Transfection of Phoenix Cells.

In situ hybridization. Synthetic digoxigenin-labeled riboprobes (cRNA) were produced based on a pcDNA3 plasmid containing corresponding base pairs of the indicated mRNA. In vitro transcription was done from both sides with either SP6 or T7 RNA polymerase, generating antisense or sense (control) cRNA probes. The probes were alkaline hydrolyzed to an average length of 200-400 bases. In situ hybridization was performed using cRNA probes for GPR37 on adult mouse brain cryosections as previously described²⁷.

LacZ staining. For LacZ staining, mice were anesthetized with ketamine/xylazine, injected intraperitoneally and perfused with 1% paraformaldehyde (PFA) (pH 7.4). Tissues were collected and postfixed with 1% PFA in 30% sucrose/PBS overnight at 4° C., followed by two hours of fixation with 0.5% glutaraldehyde in 30% sucrose/ PBS at 4° C. Tissues were washed with 30% sucrose/ PBS. Brains were embedded in OCT and sectioned (40-μm sections) on a freezing microtome, and sections were stored free-floating in PBS. Samples were washed once with wash solution (20 mM Tris-HCl (pH 7.3), 0.01% sodium deoxycholate, 0.02% NP40 and 2 mM MgCl₂) and immediately used for LacZ staining. LacZ staining was performed overnight at 37° C. in staining solution (20 mM Tris-HCl (pH 7.3), 5 mM potassium ferrocyanide, 5 mM potassium ferricyanide, 0.01% sodium deoxycholate, 0.02% NP40, 2 mM MgCl₂ and 1 mg/ml X-gal). Tissues were washed once in wash solution, transferred to glass slides, and mounted with mounting medium. Optic nerves, sciatic nerves and spinal cords were stained as a whole mount after fixation.

Antibodies and reagents: The following antibodies were used: mouse monoclonal antibodies to β gal (1:1000, G8021, Sigma), pERK (1:50, sc-7383, Santa Cruz), CC1 (1:50, OP80, Millipore), neurofilament (1:1000, NE1017, Millipore), and rabbit polyclonal antibodies to Olig2 (1:500, AB9610, Millipore), ki67 (1:500, SP6, Cell Marque), Caspr (1:1000) ⁶⁸, pERK (1:1000, 4370P, Cell signaling), ERK (1:2000, M5670, Sigma), and rat monoclonal antibodies to MBP (1:300, MAB386, Chemicon), PDGFRα (1:1000, APAS, BD Pharmingen), PLP (AA3), GPR37 (1:50, Santa Cruz, sc-27548), and GPR17 (1:20, Cayman Chemical, 17087), and hybridoma supernatants of mouse anti-O4 (1:5) and rat anti-PLP (1:10, AA3). Secondary antibodies were obtained from Jackson Immunoresearch and Invitrogen. PLX4032⁴¹ and myr-EPE⁴⁰ are generous gifts from Prof. Rony Seger (Weizmann Institute of Science, Rehovot, Israel). SQ 22536, IBMX, and ESI-09 were purchased from Sigma. cAMP levels were determined by ELISA according to the manufacturer's protocol (cAMP ELISA kit, Enzo Life Sciences).

Immunofluorescence and Immunoblotting For immunohistochemistry, mice were anesthetized and perfused with 2% PFA/PBS. Brains and optic nerves were isolated and post fixed on ice for 15 min, followed by 30% sucrose/ PBS at 4° C. overnight. Tissues were embedded in OCT and sectioned. Sections were permeabilized in methanol at −20° C. for 5 min. Immunostaining of GPR37 was carried out using 2%PFA-perfused mouse brain sections that were permeabilized with cold acetone and blocked with 5% fish gelatin and 0.5% Triton-X100 at RT for 1 hr. For immunocytochemistry, cells were fixed with 4% PFA/ PBS for 15min at room temperature. For O4 staining, live cells were incubated with medium containing the O4 antibody at 37° C. for 45min before fixation. For both immunohistochemistry and immunocytochemistry, samples were washed with PBS and blocked with PBS containing 5% normal goat serum, 0.5% Triton X-100, 0.05% sodium azide for lh. Samples were incubated overnight at 4° C. with primary antibodies diluted in blocking solution, washed three times in PBS, incubated for lh with secondary antibodies, washed in PBS and mounted with Elvanol. Images were obtained using an Axio Imager Z1 equipped with Apotome (Carl Zeiss), LSM700 confocal microscope (Carl Zeiss), or Pannoramic digital slide scanner (3DHISTECH). For image analysis, images were taken in equivalent spatial distribution from all slides. Image analysis was performed using Volocity 4.2.1 image analysis software (Perkin-Elmer) and ZEN 2011 software (Carl Zeiss). Western blot analysis was done using OPC cultures lysed in loading buffer and chemiluminescence was detected using the ChemiDoc MP System (Bio-Rad). Image (FIG. 5C) has been cropped for presentation (full size image is shown in FIG. 17.

Cell cultures. For co-cultures, mouse dorsal root ganglion (DRG) neuronal cultures and mouse glia mixed cultures of each genotype were prepared separately in advance. DRG neurons were prepared as described previously⁶⁹. Glial mixed cultures were prepared from P0-P2 mouse cortices on PDL-coated flasks and maintained in DMEM/F-12 containing 10% fetal bovine serum, 5% horse serum and penicillin-streptomycin. After 9-10 days, oligodendrocytes isolated by shaking were seeded on DRG neuronal cultures and maintained in coculture medium (DMEM containing B-27 supplement, N-2 supplement, 5 μg ml⁻¹ N-Acetyl-Cysteine, 5 μM forskolin and penicillin-streptomycin). The medium was changed every other day. OPC cultures were prepared as described above on PLD and poly-L-ornithine-coated coverslips. OPC cultures were maintained in Sato medium (DMEM containing B-27 supplement, Glutamax, penicillin-streptomycin, 1% horse serum, sodium pyruvate, 0.34 μg ml⁻¹ T3 and 0.4 μg ml⁻¹ T4). For the first two days, PDGF (final 25 ng ml⁻¹) was added to the Sato medium.

Electron microscopy. Mice were anesthetized and perfused with a fixative containing 4% PFA, 2.5% glutaraldehyde and 0.1 M cacodylate. Brains and spinal cords were isolated and incubated in the fixative overnight at room temperature and processed as previously described⁶⁸. Samples were examined using a Philips CM-12 transmission electron microscope. The EM micrographs were analyzed using computer-assisted analysis software (analysis®, Soft Imaging System) for axon diameter and total outer axon diameter containing myelin. G-ratio was calculated by dividing the measured inner axonal diameter to the measured total outer axonal diameter.

Statistical analyses. All graph data are presented as the mean ±standard of error (SEM). Statistical analyses were performed using an unpaired Student's t-test with two tails, unequal variance. Images were scored blinded to genotype before quantification. For in vivo experiments and in vitro cultures, mice were prepared as pairs before the experiments. Sample size was not predetermined, but was based on similar studies in the field.

Results

Gpr37 is an Oligodendrocyte-Enriched Gene

To examine the expression of GPR37, in situ hybridization of adult rat brains was performed (FIGS. 1A-C). GPR37 expression was mainly detected in white matter areas such as the caudate putamen, corpus callosum, hippocampal fimbria and cerebellum. To distinguish between the expression of GPR37 in oligodendrocytes versus other cell types, RT-PCR analysis of brain mRNA isolated from wild type and R26;lacZbpA(flox)DTA mice in which oligodendrocytes were eliminated using a binary genetic system was performed ^{27,28}. In support of the in situ results, the expression of GPR37 was markedly reduced after genetic depletion of oligodendrocytes (FIG. 1D). In contrast, the amount of the related receptor GPR37L1 was unchanged in the absence of oligodendrocytes, consistent with its expression in astrocytes³⁰. To further characterize the spatial and temporal expression of GPR37, the B6.129P2-Gpr37^{tm/Dgen}/J mouse line was used (hereafter referred to as Gpr37^{-/-}). This line contains a bacterial LacZ reporter gene in the Gpr37 locus ³². β-galactosidase (βgal) staining of brain slices revealed a strong expression of GPR37 in white matter fiber tracts such as the cerebellum, corpus callosum, anterior commissure, fimbria and cerebral peduncle (FIGS. 1E-G). The expression of GPR37 in hippocampal neurons was also noted. A strong signal was detected in the optic nerve (FIG. 1H) and spinal cord (FIG. 1J), but not in the sciatic nerve (FIG. 1I) or the spinal roots (FIG. 1J). Immunolabeling of P12 mice caudate putamen using antibodies to βgal and Olig2 revealed that Gpr37 was indeed present in Olig2 positive cells (FIGS. 1K-M). Notably, the intensity of βgal was inversely correlated with Olig2, which is downregulated during oligodendrocyte differentiation⁸. Developmental analysis of the cerebellum and optic nerves demonstrated that the expression of GPR37 gradually increased as myelination progressed (FIGS. 1N-O). RT-PCR and quantitative real time PCR analysis of mouse brains revealed that GPR37 transcript could be detected already at P3, and steadily increased to adulthood (FIG. 1P). This pattern of expression was distinct from Gpr17, which decreases at P20 (FIG. 1Q). Furthermore, immunolabeling of P12 brain stem revealed that the expression of these two GPCRs is mutually exclusive, (i.e., only about 4% of the cells expressing either Gpr37 or Gp17 cells were positive for both GPCRs) (FIGS. 7A-E). While Gpr17 is mainly detected in OPCs and early-differentiated cells, GPR37 is mainly present in more mature cells that do not express GPR17. These results show that GPR37 is highly expressed in myelinating glia in the CNS, that its expression increased with myelination, and persists in mature myelinating oligodendrocytes in the adult.

Absence of GPR37 Leads to Accelerated Oligodendrocyte Differentiation

During development, oligodendrocyte precursor cells differentiate along a defined pathway, marked by a typical change in cell morphology and expression of myelin-specific lipids and proteins³⁴. To begin evaluating the role GPR37 plays in CNS myelination, differentiation of wild type and Gpr37^{-/-} oligodendrocytes cultured with wild type dorsal root ganglion (DRG) neurons was followed. Antibodies to Olig2, O4 and proteolipid protein (PLP) were used as early, mid and late stage differentiation markers, respectively (FIG. 2A). Three (DIV3), or seven (DIV7) days after plating, the number of Olig2-positive cells was not significantly different between wild type and Gpr37^{-/-} cultures (FIG. 2B), indicating that GPR37 is not required for the generation of OPCs. Accordingly, the absence of GPR37 did not increase cell proliferation, as evaluated by immunolabeling for Ki-67 at DIV3 (FIG. 2C). At both time points, the percentage of Olig2 cells expressing O4 was not significantly different between the two genotypes (FIG. 2D), suggesting that the early differentiation of OPCs was not affected by the deletion of GPR37. In contrast, the number of O4-positive cells already expressing PLP was significantly higher in the Gpr37^{-/-} cultures at DIV3 (FIG. 2E). At DIV7, although the number of PLP expressing oligodendrocytes was similar in the two cultures (FIG. 2E), the intensity of PLP-immunoreactivity was >2.5 fold higher in the Gpr37^{-/-} culture (FIGS. 2F and 2A right panel). Similarly, acute shRNA knockdown of GPR37 in OPC cocultured with DRG neurons resulted in enhanced oligodendrocyte differentiation (FIGS. 8A-D).

To further assess the difference between wild type and Gpr37^{-/-} cultures, the present inventors examined the distribution of the axonal protein Caspr, which is clustered upon oligodendrocyte contact and ensheathment ^{35,36} (a process referred to as “Caspr mirroring”, which occurs just prior to myelination and formation of the paranodal junction). Immunolabeling of DIV9 cultures using antibodies to Caspr and PLP demonstrated that although the number of PLP cells was similar in both genotypes, the number of PLP-positive oligodendrocytes that were associated with intense axonal clustering of Caspr was significantly higher in Gpr37^{-/-} than in wild type co-cultures (FIGS. 2G-H). Finally, immunolabeling of OPC/DRG neurons co-cultures at DIV9 with an antibody to PLP revealed that at this stage oligodendrocytes lacking GPR37 formed longer myelin internodes than wild type cells (FIGS. 21-J). In summary, these results suggest that GPR37 negatively regulates the late-stage differentiation of oligodendrocytes.

Gpr37^{-/-} Mice Exhibits Precocious and Hypermyelination

To examine whether the faster differentiation displayed by Gpr37^{-/-} oligodendrocyte affects myelination in vivo, sagittal sections of P9 mouse brains were labeled with an antibody to myelin basic protein (MBP) (FIG. 3A). Sagittal sections were used since myelination proceeds in a caudal (posterior) to rostral (anterior) direction during brain development. In Gpr37^{-/-}, intense myelin formation in rostral brain regions such as the corpus callosum was observed, which only began to myelinate in wild type mice at this developmental stage (FIGS. 3A-B). Crossing the Gpr37^{-/-} mutant with a PLP-dsRed transgenic mouse (expressing a red fluorescence protein under the PLP promoter³⁷) allowed the present inventors to compare the fluorescence signal between PLP-dsRed and PLP-dsRed/Gpr37^{-/-}. In the optic nerve at P12, PLP-derived fluorescence signal was detected in CC1 positive oligodendrocytes in PLP-dsRed/Gpr37^{-/-} but not in PLP-dsRed control mice (FIGS. 3C-E). Consistent with the role of GPR37 in late oligodendrocyte differentiation, neither the number of PDGFRa positive OPCs, nor their proliferation increased in Gpr37^{-/-} optic nerve (FIGS. 9A-B). Electron microscopy analysis of corpus callosum at P14 revealed that GPR37^{-/-} mice exhibit a significantly higher number of myelinated axons (FIGS. 3F, 3H), and thicker myelin profiles (i.e., lower g ratio) than their wild type controls (FIGS. 3G, 3I-J). These results show that the absence of GPR37 results in accelerated myelination.

To determine whether the advanced myelination detected during development in Gpr37^{-/-} mice persisted into adulthood, the corpus callosum of wild type and mutant mice at two, four and eighteen months of age was examined by electron microscopy (FIGS. 4A-I). At all ages examined, an increase in myelin thickness with no increase in the diameter of myelinated axons was noted. This was reflected by a significant decrease in g-ratio in Gpr37^{-/-} compared to wild type mice (FIGS. 4B-C, E-F, H-I). Average g-ratios obtained were WT 0.76±0.005 vs. Gpr37^{-/-} 0.72±0.004 at 2 months; WT 0.76±0.003 vs. Gpr37^{-/-} 0.70±0.003 at 4 months, and WT 0.77±0.004 vs. Gpr37^{-/-} 0.70±0.006 at 1.5 years of age. The reduction in g-ratio in Gpr37^{-/-} brains was also observed in spinal cords of mice of the same ages (FIGS. 10A-I). These results demonstrate that the absence of GPR37 results in hypermyelination, further supporting its role as a negative regulator of myelination in the CNS.

GPR37 Signaling is Mediated by ERK

Previous studies have shown that the extracellular signal-regulated kinase (ERK1/2)/MAPK pathway regulates oligodendrocyte development and myelin thickness³⁸. Given the role this signaling system plays in mediating the action of G protein coupled receptors ³⁹, the present inventors sought to compare ERK1/2 phosphorylation in wild type and Gpr37^{-/-} brains. Immunofluorescence labeling of P12 brain stem sections using antibodies to Olig2 and phosphorylated ERK1/2, revealed an intense phospho-ERK immunoreactivity in the mutant, but not in the wild type controls (FIGS. 5A-B). A significant increase in ERK phosphorylation was also evident by Western blot analysis of cultured oligodendrocytes lacking GPR37 (FIGS. 5C-D). As one of the key steps in ERK1/2 signaling is their nuclear translocation ⁴⁰, the present inventors immunolabeled cultured oligodendrocytes isolated from wild type or Gpr37^{-/-} mice using antibodies to PLP and phospho-ERK1/2 (FIG. 5E). It was noted that the number of Gpr37^{-/-} oligodendrocytes exhibiting nuclear localization of pERK1/2 was double than the wild type cells (FIG. 5F). These results indicate that the absence of GPR37 results in enhanced ERK1/2 phosphorylation and translocation to the nucleus.

To assess whether the effect of GPR37 on oligodendrocyte differentiation is mediated by MAPK signaling, myelinating co-cultures containing wild type or Gpr37^{-/-} oligodendrocytes were treated with PLX4032⁴¹, an inhibitor of Raf kinases, which relay extracellular signals to the MAPK module and play a role in oligodendrocyte maturation and myelination⁴². As an alternative approach, the cultures were grown in the presence of myr-EPE, a myristoylated phosphomimetic peptide that inhibits the nuclear translocation of ERK1/2⁴⁰. This peptide also prevented the accumulation of ERK1/2 in the nucleus of oligodendrocytes lacking GPR37 (FIG. 11A). Cells were immunolabeled for PLP and Caspr (FIG. 5H). The clustering of axonal Caspr induced by oligodendrocyte contact was used to evaluate differentiation. Remarkably, both inhibitors completely abolished the number of PLP-positive oligodendrocytes that were associated with intense axonal clustering of Caspr in Gpr37^{-/-} (FIG. 5G). PLX4032 and myr-EPE had no effect on wild type cultures (FIG. 5G), nor did they affect the number of Olig2 cells of both genotypes (FIG. 11B). In line with previous studies suggesting that GPR37 and GPR37L1 are coupled to Gα_{i/o} (i.e., inhibiting adenylate cyclase)⁴³, the present inventors detected an increase in cAMP levels in both Gpr37^{-/-} brains (FIG. 12A) and cultured Gpr37^{-/-} oligodendrocytes (FIG. 12B). Furthermore, the present inventors noted a significant decrease in the number of Gpr37^{-/-} oligodendrocytes displaying nuclear localization of ERK1/2 after treatment with either an adenylate cyclase (SQ 22536) (FIGS. 12C-D) or an EPAC (exchange protein activated by cAMP) (ESI-09) inhibitor (FIGS. 13A-B), indicating that cAMP signaling downstream of GPR37 is mediated by EPAC. As an additional indication for the involvement of cAMP in GPR37 signaling, it was found that the addition of the phosphodiesterase inhibitor 3-isobutyl-1-methylxanthine (IBMX) enhances the differentiation of wild type oligodendrocyte to a level comparable to that observed in Gpr37^{-/-} cells (FIGS. 14A-B).

As the differentiation of the oligodendrocytes is controlled by a complex transcriptional network^{60,61}, the present examine whether the absence of GPR37 affects the expression of twelve transcription factors. They performed quantitative real time PCR of P4 brain stem RNA isolated from Gpr37^{-/-} and their littermate wild type control mice (FIG. 15). This analysis revealed that the absence of Gpr37 lead to increased expression of the stimulatory myelin regulatory factor (Myrf), which activates myelin gene expression genes⁶²⁻⁶⁴. It also resulted in a decrease in the level of the inhibitory bHLH factor Hes5⁶⁶, which negatively regulates Myrf⁶⁷. Taken together, these results indicate that the inhibitory action of GPR37 in oligodendrocytes is mediated by the suppression of cAMP-dependent inhibition of ERK1/2 phosphorylation, its nuclear translocation and attenuation of Myrf expression.

Conclusions

Previous studies have shown that GPR17 negatively regulates oligodendrocyte differentiation and myelination^{12,46}. GPR17 is expressed at the transition from late OPCs (also termed pre-oligodendrocytes) to immature pre-oligodendrocytes, but is down regulated in myelinating oligodendrocytes^{12,47}. Such a pattern of expression is distinct from GPR37, which appears in pre-myelinating oligodendrocytes, and continues to be expressed in myelinating oligodendrocytes in the adult. Moreover, double-labeling experiments of brain stem at P12, revealed that the number of cells expressing both GPR17 and GPR37 is rather limited and account for only 5% of premyelinating oligodendrocytes. This difference is also reflected by the different phenotypes obtained by deleting these GPCRs in mice: while the absence of either GPR17 or GPR37 resulted in premature formation of myelinating oligodendrocytes during development, myelination of adult GPR17^{-/-} mice was comparable to wild type, whereas the absence of GPR37 results in persistent hypermyelination. These results suggest that although both GPR17 and GPR37 negatively regulate oligodendrocyte differentiation, they act at different stages of development of the lineage. Interestingly, in addition to GPR17 and GPR37, it was recently shown that the adhesion-type GPR56 affects oligodendrocyte differentiation by positively regulating OPCs proliferation^{21,22}. Overexpression of GPR56 causes an increase in the number of OPCs and thus inhibition of myelination²¹, whereas its absence causes a reduction in the number of OPCs, and as a result premature differentiation and hypomyelination^{21,22}. Given the differential expression of GPR56, GPR17 and GPR37 during development, as well as the unique role they play in regulating OPCs proliferation (GPR56), early (GPR17) and late (GPR37) stages of oligodendrocyte differentiation, the present inventors propose that the development of the oligodendrocyte lineage from OPCs to myelinating cells is controlled by the sequential activity of inhibitory GPCRs (FIG. 6A).

Gain- and loss-of-function studies have linked ERK1/2 signaling to the regulation of myelin thickness in both CNS^{38,45} and PNS⁵⁶. Entirely consistent with these results, we found that the absence of GPR37 caused an increase in ERK1/2 phosphorylation in both cultured oligodendrocytes and mouse brains. Pharmacological inhibition of Raf kinases, which are required for the sequential activation of MEK1/2 and ERK1/2³⁹, prevented the premature differentiation of Gpr37^{-/-} oligodendrocytes without affecting their proliferation. Furthermore, a similar effect was obtained by treatment of Gpr37^{-/-} oligodendrocytes with the myr-EPE peptide, which inhibits the nuclear translocation of ERK1/2⁴⁰. Nuclear translocation of ERK1/2 in Gpr37^{-/-} oligodendrocytes was also attenuated by pharmacological inhibition of adenylate cyclase, suggesting that GPR37 signaling is normally suppresses cAMP levels. This notion is supported by the observation that oligodendrocytes and mouse brains lacking GPR37 contain higher levels of cAMP compared to their wild type controls. In line with these observations, cAMP was shown to stimulate the expression of myelin genes such as MBP and PLP, and the maturation of oligodendrocytes⁵⁷. Similarly, inhibition of the cAMP-hydrolyzing enzyme phosphodiesterase 4 (Pde4) promoted MAPK signaling and OPC differentiation^{58,59}. Overall, these results suggest that GPR37 controls late stage oligodendrocyte differentiation, at least in part, by suppressing cAMP, which further leads to the downstream inhibition of ERK1/2 activation and nuclear translocation (FIG. 6B).

### EXAMPLE 2

### Effect of G Protein Coupled Receptor 37 in a Mouse Model for Multiple Sclerosis Materials and methods

Female and male C57BL/6Jmice of 9-12 weeks of age were used (Total mouse number=14 WT and 20 KO, (Gpr37^{-/-}), Female mouse number=4WT and 7KO, Male mouse number=10 WT and 13 KO). Chronic EAE was induced in mice by injecting a peptide consisting of amino acids 35-55 of myelin oligodendrocyte glycoprotein (MOG). Mice were injected subcutaneously at the flank, with 200 μl emulsion containing 200-300 μg of the encephalitogenic peptide in incomplete Freund's adjuvant enriched with 3 mg/ml heat-inactivated Mycobacterium tuberculosis (Sigma, St. Louis, Mo., USA). Pertussis toxin (Sigma), 250 μg/mouse, was injected intravenously immediately after the encephalitogenic injection and 48 h later. Mice were examined daily. EAE was scored as follows: 0—no disease, 1—limp tail, 2—hind limb paralysis, 3—paralysis of all limbs, 4—moribund condition, and 5—death.

Results

As illustrated in FIG. 18, Gpr37 knock-out mice showed a lower clinical score compared to wild-type mice following induction of EAE. The Gpr37 knock-out mice exhibited a faster recovery after EAE as compared to wild-type mice.

Electron microscopy micrographs of wild type and Gpr37 KO spinal cord 19 days after the induction of EAE portray massive demyelination and axonal loss in WT but not in the KO (which likely underwent complete remyelination) (FIGS. 19A-B).

### Example 3

Materials and Methods

Mice. Gpr37^{-/-} (B6.129P2-Gpr37^{tmlDgen}/J) mice were obtained from the Jackson Laboratory and were maintained on a C57BL/6J background. Age and gender of mice used for experiments is detailed in the figure legends. Mice were genotyped for the targeted allele by PCR using tail DNA. For the targeted allele, the forward primer in neo 5′-gggtgggattagataaatgcctgctct-3′ (SEQ ID NO: 58) and the gene-specific reverse primer 5′-ggccaagagagaattggagatgctc-3′ (SEQ ID NO: 59) were used. For the endogenous allele, the gene-specific forward primer 5′-aacgggtctgcagatgactgggttc-3′ (SEQ ID NO: 60) and the gene-specific reverse primer 5′-ggccaagagagaattggagatgctc-3′ (SEQ ID NO: 61) were used.

Lysolecithin-induced demyelination in the corpus callosum. Lysolecithin injection was performed as described previously (Feng et al., J. Neurosci. 36 (30) 7925-7935 (2016)). Briefly, demyelinated lesions were induced in the corpus callosum of the brain of 8-week-old Gpr37^{-/-}, and their control littermate wild-type mice. The animals were anesthetized with isoflurane, and positioned in a stereotaxic frame. A Hamilton syringe (65458-01, 7001, 1 μl) was mounted on a stereotaxic micromanipulator. Focal demyelination was induced by stereotaxic injection of 1 μl of a solution of 1% LPC (_{L}-a-lysophosphatidylcholine, Sigma-Aldrich) in PBS. The demyelinating agent was injected unilaterally into the corpus callosum using stereotaxic coordinates of 1.04 mm posterior to the bregma, 1 mm lateral, and 1.75 mm deep from the skull surface. The needle was kept in place for 5 minutes to reduce reflux along the needle track. After lesioning, mice were allowed to recover as specified in the Figures.

Experimental autoimmune encephalomyelitis (EAE). Chronic EAE was induced in Gpr37^{-/-} and littermate WT mice at the age of 9-12 weeks by injecting a peptide consisting of amino acids 35-55 of myelin oligodendrocyte glycoprotein (MOG), which was synthesized by Genscript (Piscataway, N.J., USA). Mice were injected subcutaneously at the flank, with 200 μl emulsion containing 200-300 μg of the encephalitogenic peptide in incomplete Freund's adjuvant enriched with 3 mg/ml heat-inactivated Mycobacterium tuberculosis (Sigma, St. Louis, Mo., USA). Pertussis toxin (Sigma), 250 μg/mouse, was injected intravenously immediately after the encephalitogenic injection and 48 h later. Mice were examined daily. EAE was scored as follows: 0—no disease, 1—limp tail, 2—hind limb paralysis, 3—paralysis of all limbs, 4—moribund condition, and 5—death.

Immunohistochemistry and other histology stainings. For immunohistochemistry in lysolecithin model, mice were anaesthetized and perfused with 4% PFA/PBS. Brains were isolated and post-fixed on ice for 15 min, followed by 30% sucrose/PBS at 4° C. overnight. Tissues were embedded in OCT and sectioned in 15 μm and dried for 1-2 h. Sections were washed with PBS three times and permeabilized in precooled methanol at −20° C. for 2 min. Sections were washed with PBS three times and blocked with 10% Normal goat serum (NGS)/0.1% Triton-X100/0.3M Glycine/0.02% NaN₃/PBS at room temperature (RT) for 1 h, followed by primary incubation in 5% NGS/0.3M Glycine/0.02% NaN₃/PBS at 4° C. overnight. On the next day, sections were washed three times with PBS and incubated with fluorescent secondary antibodies diluted in 5% NGS/0.3M Glycine/0.02% NaN3/PBS at RT for 1 h, followed by PBS washing three times. Sections were cleaned and mounted with Elvanol™. For immunohistochemistry in EAE and cuprizone assay, mice were anaesthetized and perfused with 2% PFA/PBS and isolated tissues were preserved in 1% PFA/PBS until the processing of making paraffin blocks. For spinal cord tissues in EAE, decalcification was performed after the perfusion by immersing them in the 12.5% EDTA (pH 7.2-7.3) at RT for 3-5 days with daily buffer change. Paraffin sections were sectioned in 4 μm and heat-induced antigen retrieval was performed at pH 6, 8 or 9 depending on the antibodies for immunostaining. Paraffin sections were stained with H&E to see the nuclei and with Luxol fast blue to label myelin structure.

Electron microscope. Mice were anaesthetised and perfused with a fixative containing 4% PFA, 2.5% glutaraldehyde and 0.1M cacodylate. Brains and spinal cords were isolated and incubated in the fixative overnight at room temperature and processed as previously described68. Samples were examined using a Philips CM-12 transmission electron microscope. The EM micrographs were analyzed using computer-assisted analysis software (analysis, Soft Imaging System).

Antibodies. The following antibodies for immunostaining were used: mouse monoclonal antibodies to Ermin(1:50), GFAP(1:200), CNP (1:200) and rabbit polyclonal antibodies to Olig2 (1:500, AB9610, Millipore), GPR17(1:20, Cayman Chemical, 17087), Ermin (1:100), Caspr(1:1000, 6062, Poliak et al., 2003), CD3(1:50), Opalin (1:100), Iba (1:200, Wako) and rat monoclonal antibodies to MBP (1:300, MAB386, Chemicon), PDGFRα (1:1000, APAS, BD Pharmingen), PLP (1:10, AA3), Mac2 (1:150).

Results

To examine whether GPR37 is inhibitory to remyelination, two different experimental demyelination models were used: i. local demyelination in the corpus callosum induced by lysolecithin, and ii. experimental autoimmune encephalomyelitis (EAE).

i. Absence of Gpr37 Results in Enhanced Remyelination of Focal Demyelination Lesion.

Mice were sacrificed at 3 and 12 days post-lysolecithin injection (dpi). At 3 dpi, there was a significant accumulation of cells as reflected by an increase in the number of nuclei in the lesioned area in both WT and Gpr37^{-/-} tissues (FIG. 20A,B). At this time point there was no significant difference in the intensity of MBP (FIG. 20A,C). At 12 dpi, cell accumulation was slightly increased compared to 3 dpi in both WT and Gpr37^{-/-} tissues (FIG. 20A,E). In contrast to 3 dpi, at 12 dpi, MBP density within the dapi-accumulated area was significantly reduced in the lesioned area compared to the intact area in both genotypes (FIGS. 20A,F). The lesioned area was similar in size in both genotypes at 3 and 12 dpi (FIGS. 20D,G). Staining for early stage markers of oligodendrocyte differentiation such as Olig2, PDGFRα and GPR17 at 3 dpi showed no significant changes between the genotypes (FIGS. 21A-D). However, there were significant changes between WT and Gpr37^{-/-} tissues in the late stage OL markers such as PLP and Ermin (FIGS. 22A-B) and MBP (FIG. 20F). At 12 dpi, there was a clear increase in the number of Caspr-labeled paranodal junction in the lesioned area in Gpr37^{-/-} compared to WT control (FIGS. 23A-E), further indicating that enhanced remyelination occurs in the absence of GPR37. At 12 dpi, there were many more PDGFRa-positive cells in the lesion site of wild type compared to Gpr37^{-/-} (FIGS. 24A-C), suggesting that newly appeared oligodendrocyte cells mature faster in the absence of GPR37. In summary, it was found that oligodendrocyte differentiation and remyelination is faster and more robust in Gpr37^{-/-} compared to wild type counterparts.

ii. The Absence of Gpr37 Prevents the Chronic Progression of EAE Pathology and Induces Remyelination and Repair.

To induce experimental autoimmune encephalomyelitis (EAE), a murine model of multiple sclerosis was used in which MOG peptide was injected into 9-12 weeks old mice. Intriguingly, the clinical score of Gpr37^{-/-} was dramatically reduced compared to the score of wild type animals. The scores of Gpr37^{-/-} were significantly lower than WT from day 18 forward (FIGS. 25A-B). H&E staining showed a significant increase in the accumulation of immune cells nuclei between day 12 and day 19 in both WT and Gpr37^{-/-} tissues (FIG. 26G). However, it appears that inflammation was different between the genotypes after day 19: in Gpr37^{-/-} tissues, the number of nuclei were reduced dramatically by day 30, compared to day 19, while no significant changes were observed in WT tissues (FIG. 26G). To further examine the immune system, tissue sections were labeled using antibodies to Ibal (for labeling microglia), Mac2 (for labeling activated microglia) and to CD3 (for labeling T cells). An increase in the number of Iba⁺ (or Mac2⁺) cells in lumbar spinal cords was noted in wild type but not in Gpr37^{-/-} mice (FIGS. 27A-D and 28A-C). Compared to microglia, T cell number (CD3^{⇄}) gradually increased in both genotypes, but there was a dramatic reduction in the number of CD3⁺ cell between day 19-30 only in Gpr37^{-/-} tissues (FIGS. 29A-C). Interestingly, a correlation between inflammation (indicated by number of nucleus, FIG. 26A-G) and myelin density (indicated by % LFB Area, FIG. 30A-G) was also noted. From day 15 until day 19, there were significant reductions in LFB⁺ staining in the lumbar spinal cords of both genotypes, indicating the robust demyelination during this period. However, LFB staining in significantly increased from day 19 till day 30 in KO, but not wild type tissue, indicating that extensive remyelination occurs in the absence of GPR37. Myelin density was also examined by other myelin markers such as Opalin (FIG. 31A-C) or toluidine blue (FIG. 32A-L), both showing consistent results with LFB staining (FIG. 30A-G). To further visualize myelination and demyelinated areas in the lumbar spinal cords of WT-EAE or Gpr37^{-/-} of EAE animals at day 30, sections stained with toluidine blue were analyzed (FIG. 32A-L). There was a dramatic difference in myelin between WT-EAE and Gpr37^{-/-}-EAE, especially close to the surface area of the lumbar spinal cord. The same tissues were processed for electron microscopy (FIG. 33A-F). Low-magnification images showed demyelination as well as abnormal myelin near the surface of ventral-lateral spinal cord of WT (FIG. 33A) and many thin remyelinated axons in Gpr37^{-/-} (FIGS. 33B-F) mice.

In conclusion, the absence of Gpr37 results in faster remyelination in two distinct experimental models of demyelination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Nave, K. A. & Werner, H. B. Myelination of the nervous system: mechanisms and functions. Annu Rev Cell Dev Biol 30, 503-33 (2014).
2. Liu, J. et al. Impaired adult myelination in the prefrontal cortex of socially isolated mice. Nat Neurosci 15, 1621-3 (2012).
3. Young, K. M. et al. Oligodendrocyte dynamics in the healthy adult CNS: evidence for myelin remodeling. Neuron 77, 873-85 (2013).
4. Perlman, S. J. & Mar, S. Leukodystrophies. Adv Exp Med Biol 724, 154-71 (2012).
5. Nave, K. A. & Ehrenreich, H. Myelination and oligodendrocyte functions in psychiatric diseases. JAMA Psychiatry 71, 582-4 (2014).
6. Franklin, R. J. & Gallo, V. The translational biology of remyelination: past, present, and future. Glia 62, 1905-15 (2014).
7. Hughes, E. G., Kang, S. H., Fukaya, M. & Bergles, D. E. Oligodendrocyte progenitors balance growth with self-repulsion to achieve homeostasis in the adult brain. Nat Neurosci 16, 668-76 (2013).
8. Mitew, S. et al. Mechanisms regulating the development of oligodendrocytes and central nervous system myelin. Neuroscience 276, 29-47 (2014).
9. Charles, P. et al. Negative regulation of central nervous system myelination by polysialylated-neural cell adhesion molecule. Proc Natl Acad Sci USA 97, 7585-90 (2000).
10. Fancy, S. P. et al. Dysregulation of the Wnt pathway inhibits timely myelination and remyelination in the mammalian CNS. Genes Dev 23, 1571-85 (2009).
11. Mi, S. et al. LINGO-1 negatively regulates myelination by oligodendrocytes. Nat Neurosci 8, 745-51 (2005).
12. Chen, Y. et al. The oligodendrocyte-specific G protein-coupled receptor GPR17 is a cell-intrinsic timer of myelination. Nat Neurosci 12, 1398-406 (2009).
13. Wang, S. et al. Notch receptor activation inhibits oligodendrocyte differentiation. Neuron 21, 63-75 (1998).
14. Colognato, H. et al. CNS integrins switch growth factor signalling to promote target-dependent survival. Nat Cell Biol 4, 833-41 (2002).
15. Xiao, J. et al. Brain-derived neurotrophic factor promotes central nervous system myelination via a direct effect upon oligodendrocytes. Neurosignals 18, 186-202 (2010).
16. Furusho, M., Dupree, J. L., Nave, K. A. & Bansal, R. Fibroblast growth factor receptor signaling in oligodendrocytes regulates myelin sheath thickness. The Journal of neuroscience : the official journal of the Society for Neuroscience 32, 6631-41 (2012).
17. Ahrendsen, J. T. & Macklin, W. Signaling mechanisms regulating myelination in the central nervous system. Neurosci Bull 29, 199-215 (2013).
18. Mogha, A. et al. Gpr126 functions in Schwann cells to control differentiation and myelination via G-protein activation. J Neurosci 33, 17976-85 (2013).
19. Monk, K. R. et al. A G protein-coupled receptor is essential for Schwann cells to initiate myelination. Science 325, 1402-5 (2009).
20. Trimarco, A. et al. Prostaglandin D2 synthase/GPR44: a signaling axis in PNS myelination. Nat Neurosci 17, 1682-92 (2014).
21. Ackerman, S. D., Garcia, C., Piao, X., Gutmann, D. H. & Monk, K. R. The adhesion GPCR Gpr56 regulates oligodendrocyte development via interactions with Galphal2/13 and RhoA. Nat Commun 6, 6122 (2015).
22. Giera, S. et al. The adhesion G protein-coupled receptor GPR56 is a cell-autonomous regulator of oligodendrocyte development. Nat Commun 6, 6121 (2015).
23. Piao, X. et al. Genotype-phenotype analysis of human frontoparietal polymicrogyria syndromes. Ann Neurol 58, 680-7 (2005).
24. Deshmukh, V.A. et al. A regenerative approach to the treatment of multiple sclerosis. Nature 502, 327-32 (2013).
25. Hammond, T. R. et al. Astrocyte-derived endothelin-1 inhibits remyelination through notch activation. Neuron 81, 588-602 (2014).
26. Yuen, T. J. et al. Identification of endothelin 2 as an inflammatory factor that promotes central nervous system remyelination. Brain 136, 1035-47 (2013).
27. Golan, N. et al. Identification of Tmem10/Opalin as an oligodendrocyte enriched gene using expression profiling combined with genetic cell ablation. Glia 56, 1176-86 (2008).
28. Brockschnieder, D., Sabanay, H., Riethmacher, D. & Peles, E. Ermin, a myelinating oligodendrocyte-specific protein that regulates cell morphology. J Neurosci 26, 757-62 (2006).
29. Marazziti, D., Gallo, A., Golini, E., Matteoni, R. & Tocchini-Valentini, G. P. Molecular cloning and chromosomal localization of the mouse Gpr37 gene encoding an orphan G-protein-coupled peptide receptor expressed in brain and testis. Genomics 53, 315-324 (1998).
30. Zhang, Y. et al. An RNA-sequencing transcriptome and splicing database of glia, neurons, and vascular cells of the cerebral cortex. J Neurosci 34, 11929-47 (2014).
31. Imai, Y. et al. An unfolded putative transmembrane polypeptide, which can lead to endoplasmic reticulum stress, is a substrate of parkin. Cell 105, 891-902 (2001).
32. Roesch, K. et al. The transcriptome of retinal Muller glial cells. J Comp Neurol 509, 225-38 (2008).
33. Zhang, Y. et al. Purification and characterization of progenitor and mature human astrocytes reveals transcriptional and functional differences with mouse. Neuron In press(2015).
34. Barateiro, A. & Fernandes, A. Temporal oligodendrocyte lineage progression: in vitro models of proliferation, differentiation and myelination. Biochim Biophys Acta 1843, 1917-29 (2014).
35. Eisenbach, M. et al. Differential clustering of Caspr by oligodendrocytes and Schwann cells. J Neurosci Res 87, 3492-501 (2009).
36. Pedraza, L., Huang, J.K. & Colman, D. Disposition of axonal caspr with respect to glial cell membranes: Implications for the process of myelination. J Neurosci Res 87, 3480-91 (2009).
37. Hirrlinger, P.G. et al. Expression of reef coral fluorescent proteins in the central nervous system of transgenic mice. Mol Cell Neurosci 30, 291-303 (2005).
38. Ishii, A., Fyffe-Maricich, S.L., Furusho, M., Miller, R.H. & Bansal, R. ERK1/ERK2 MAPK signaling is required to increase myelin thickness independent of oligodendrocyte differentiation and initiation of myelination. The Journal of neuroscience : the official journal of the Society for Neuroscience 32, 8855-64 (2012).
39. Keshet, Y. & Seger, R. The MAP kinase signaling cascades: a system of hundreds of components regulates a diverse array of physiological functions. Methods Mol Biol 661, 3-38 (2010).
40. Plotnikov, A. et al. The nuclear translocation of ERK1/2 as an anticancer target. Nat Commun 6, 6685 (2015).
41. Sala, E. et al. BRAF silencing by short hairpin RNA or chemical blockade by PLX4032 leads to different responses in melanoma and thyroid carcinoma cells. Mol Cancer Res 6, 751-9 (2008).
42. Galabova-Kovacs, G. et al. Essential role of B-Raf in oligodendrocyte maturation and myelination during postnatal central nervous system development. J Cell Biol 180, 947-55 (2008).
43. Meyer, R.C., Giddens, M.M., Schaefer, S.A. & Hall, R.A. GPR37 and GPR37L1 are receptors for the neuroprotective and glioprotective factors prosaptide and prosaposin. Proceedings of the National Academy of Sciences of the United States of America (2013).
44. Brinkmann, B.G. et al. Neuregulin-1/ErbB signaling serves distinct functions in myelination of the peripheral and central nervous system. Neuron 59, 581-95 (2008).
45. Ishii, A., Furusho, M. & Bansal, R. Sustained activation of ERK1/2 MAPK in oligodendrocytes and schwann cells enhances myelin growth and stimulates oligodendrocyte progenitor expansion. The Journal of neuroscience : the official journal of the Society for Neuroscience 33, 175-86 (2013).
46. Hennen, S. et al. Decoding signaling and function of the orphan G protein-coupled receptor GPR17 with a small-molecule agonist. Sci Signal 6, ra93 (2013).
47. Boda, E. et al. The GPR17 receptor in NG2 expressing cells: focus on in vivo cell maturation and participation in acute trauma and chronic damage. Glia 59, 1958-73 (2011).
48. Harlow, D. E. & Macklin, W. B. Inhibitors of myelination: ECM changes, CSPGs and PTPs. Exp Neurol 251, 39-46 (2014).
49. Mei, F., Christin Chong, S. Y. & Chan, J. R. Myelin-based inhibitors of oligodendrocyte myelination: clues from axonal growth and regeneration. Neurosci Bull 29, 177-88 (2013).
50. Rosenberg, S. S., Kelland, E. E., Tokar, E., De la Torre, A. R. & Chan, J. R. The geometric and spatial constraints of the microenvironment induce oligodendrocyte differentiation. Proc Natl Acad Sci USA 105, 14662-7 (2008).
51. Lee, S. et al. A culture system to study oligodendrocyte myelination processes using engineered nanofibers. Nat Methods 9, 917-22 (2012).
52. Czopka, T., Ffrench-Constant, C. & Lyons, D. A. Individual oligodendrocytes have only a few hours in which to generate new myelin sheaths in vivo. Dev Cell 25, 599-609 (2013).
53. Watkins, T. A., Emery, B., Mulinyawe, S. & Barres, B. A. Distinct stages of myelination regulated by gamma-secretase and astrocytes in a rapidly myelinating CNS coculture system. Neuron 60, 555-69 (2008).
54. Back, S. A., Luo, N. L., Borenstein, N. S., Volpe, J. J. & Kinney, H. C. Arrested oligodendrocyte lineage progression during human cerebral white matter development: dissociation between the timing of progenitor differentiation and myelinogenesis. J Neuropathol Exp Neurol 61, 197-211 (2002).
55. Meyer, R. C., Giddens, M. M., Coleman, B. M. & Hall, R. A. The protective role of prosaposin and its receptors in the nervous system. Brain Res 1585, 1-12 (2014).
56. Sheean, M. E. et al. Activation of MAPK overrides the termination of myelin growth and replaces Nrg1/ErbB3 signals during Schwann cell development and myelination. Genes Dev 28, 290-303 (2014).
57. Afshari, F. S., Chu, A. K. & Sato-Bigbee, C. Effect of cyclic AMP on the expression of myelin basic protein species and myelin proteolipid protein in committed oligodendrocytes: differential involvement of the transcription factor CREB. J Neurosci Res 66, 37-45 (2001).
58. Sun, X., Liu, Y., Liu, B., Xiao, Z. & Zhang, L. Rolipram promotes remyelination possibly via MEK-ERK signal pathway in cuprizone-induced demyelination mouse. Exp Neurol 237, 304-11 (2012).
59. Syed, Y. A. et al. Inhibition of phosphodiesterase-4 promotes oligodendrocyte precursor cell differentiation and enhances CNS remyelination. EMBO Mol Med 5, 1918-34 (2013).
60. Emery, B. & Lu, Q. R. Transcriptional and Epigenetic Regulation of Oligodendrocyte Development and Myelination in the Central Nervous System. Cold Spring Harb Perspect Biol 7, a020461 (2015).
61. Hernandez, M. & Casaccia, P. Interplay between transcriptional control and chromatin regulation in the oligodendrocyte lineage. Glia 63, 1357-75 (2015).
62. Bujalka, H. et al. MYRF is a membrane-associated transcription factor that autoproteolytically cleaves to directly activate myelin genes. PLoS Biol 11, e1001625 (2013).
63. Emery, B. et al. Myelin gene regulatory factor is a critical transcriptional regulator required for CNS myelination. Cell 138, 172-85 (2009).
64. Koenning, M. et al. Myelin gene regulatory factor is required for maintenance of myelin and mature oligodendrocyte identity in the adult CNS. J Neurosci 32, 12528-42 (2012).
65. Ishii, A., Furusho, M., Dupree, J. L. & Bansal, R. Role of ERK1/2 MAPK signaling in the maintenance of myelin and axonal integrity in the adult CNS. J Neurosci 34, 16031-45 (2014).
66. Liu, A. et al. A molecular insight of HesS-dependent inhibition of myelin gene expression: old partners and new players. EMBO J 25, 4833-42 (2006).
67. Hornig, J. et al. The transcription factors Sox10 and Myrf define an essential regulatory network module in differentiating oligodendrocytes. PLoS Genet 9, e1003907(2013).
68. Poliak, S. et al. Juxtaparanodal clustering of Shaker-like K+ channels in myelinated axons depends on Caspr2 and TAG-1. The Journal of cell biology 162, 1149-60 (2003).
69. Eshed, Y. et al. Gliomedin mediates Schwann cell-axon interaction and the molecular assembly of the nodes of Ranvier. Neuron 47, 215-29 (2005).

1. (canceled)

2. A method of treating a demyelinating disease of a subject in need thereof, comprising:
(a) contacting oligodendrocytes or progenitors thereof with an agent that downregulates the activity and/or amount of GPR37, wherein said agent is an antagonist to said GPR37 or wherein said agent binds to a polynucleotide encoding said GPR37 so as to generate cells with an enhanced myelinating activity; and
(b) providing said cells with an enhanced myelinating activity to said subject, thereby treating the demyelinating disease.

3. A method of treating a demyelinating disease of a subject in need thereof, comprising administering to the subject a therapeutically effective amount of an agent that downregulates the activity or amount of GPR37, wherein said agent is an antagonist to said GPR37 or wherein said agent binds to a polynucleotide encoding same, thereby treating the demyelinating disease.

4. The method of claim 2, wherein said demyelinating disease is selected from the group consisting of multiple sclerosis, optic neuritis, an idiopathic inflammatory demyelinating disease, Guillain-Barre Syndrome, chronic inflammatory demyelinating polyneuropathy, transverse myelitis, Balo concentric sclerosis, pernicious anemia, central pontine myelinolysis, Tabes dorsalis, neuromyelitis optica (NMO), progressive multifocal leukoencephalopathy (PML), anti-MAG (myelin-associated glycoprotein) neuropathy, hereditary motor and sensory neuropathy (Chacot-Marie-Tooth disease), cerebrotendinious xanthanomatosis, and leukodystrophies including adrenoleukodystrophy, adrenomyeloneuropathy, metachromatic leukodystrophy, globoid cell leukodystrophy (Krabbe disease), Canavan disease, vanishing white matter disease, Alexander disease, Refsum disease, and Pelizaeus-Merzbacher disease.

5. The method of claim 4, wherein said demyelinating disease is multiple sclerosis (MS).

6-8. (canceled)

9. The method of claim 2, wherein said agent is selected from the group consisting of a peptide agent, a small molecule agent, an antibody and a small molecule agent.

10. A method of selecting an agent which is capable of increasing the myelinating activity of an oligodendrocyte comprising:
(a) contacting cells which express GPR37 with a candidate agent; and
(b) measuring an amount and/or activity of said GPR37, wherein when said amount and/or activity is below a predetermined level, it is indicative that the candidate agent is capable of increasing the myelinating activity of an oligodendrocyte.

11. The method of claim 10, wherein said cells are oligodendrocytes or progenitors thereof.

12. The method of claim 10, wherein said cells are genetically modified to express GRP37.

13. The method of claim 3, wherein said demyelinating disease is selected from the group consisting of multiple sclerosis, optic neuritis, an idiopathic inflammatory demyelinating disease, Guillain-Barre Syndrome, chronic inflammatory demyelinating polyneuropathy, transverse myelitis, Balo concentric sclerosis, pernicious anemia, central pontine myelinolysis, Tabes dorsalis, neuromyelitis optica (NMO), progressive multifocal leukoencephalopathy (PML), anti-MAG (myelin-associated glycoprotein) neuropathy, hereditary motor and sensory neuropathy (Chacot-Marie-Tooth disease), cerebrotendinious xanthanomatosis, and leukodystrophies including adrenoleukodystrophy, adrenomyeloneuropathy, metachromatic leukodystrophy, globoid cell leukodystrophy (Krabbe disease), Canavan disease, vanishing white matter disease, Alexander disease, Refsum disease, and Pelizaeus-Merzbacher disease.

14. The method of claim 13, wherein said demyelinating disease is multiple sclerosis (MS).

15. The method of claim 3, wherein said agent is selected from the group consisting of a peptide agent, a small molecule agent, an antibody and a small molecule agent.


SEQUENCE LISTING (61 sequences)

SEQ ID NO: 1 - DNA, 27 bp, Artificial sequence
  Single strand DNA oligonucleotide
gggtgggattagataaatgcctgctct

SEQ ID NO: 2 - DNA, 25 bp, Artificial sequence
  Single strand DNA oligonucleotide
ggccaagagagaattggagatgctc

SEQ ID NO: 3 - DNA, 25 bp, Artificial sequence
  Single strand DNA oligonucleotide
aacgggtctgcagatgactgggttc

SEQ ID NO: 4 - DNA, 25 bp, Artificial sequence
  Single strand DNA oligonucleotide
ggccaagagagaattggagatgctc

SEQ ID NO: 5 - DNA, 19 bp, Artificial sequence
  Single strand DNA oligonucleotide
cctgcaagatcgtgcccta

SEQ ID NO: 6 - DNA, 21 bp, Artificial sequence
  Single strand DNA oligonucleotide
agtacatctggacgttggtgg

SEQ ID NO: 7 - DNA, 19 bp, Artificial sequence
  Single strand DNA oligonucleotide
ctttaggtgggcatagagc

SEQ ID NO: 8 - DNA, 21 bp, Artificial sequence
  Single strand DNA oligonucleotide
tggagaactggttgatgaggc

SEQ ID NO: 9 - DNA, 18 bp, Artificial sequence
  Single strand DNA oligonucleotide
tgccgctgttttggataa

SEQ ID NO: 10 - DNA, 20 bp, Artificial sequence
  Single strand DNA oligonucleotide
cgcctcggaaatagtatttg

SEQ ID NO: 11 - DNA, 20 bp, Artificial sequence
  Single strand DNA oligonucleotide
ccagagcggctgtctcttcc

SEQ ID NO: 12 - DNA, 22 bp, Artificial sequence
  Single strand DNA oligonucleotide
catccttgactccatcgggcgc

SEQ ID NO: 13 - DNA, 25 bp, Artificial sequence
  Single strand DNA oligonucleotide
gagcaccctgtgctgctcaccgagg

SEQ ID NO: 14 - DNA, 25 bp, Artificial sequence
  Single strand DNA oligonucleotide
gtggtggtgaagctgtagccacgct

SEQ ID NO: 15 - DNA, 21 bp, Artificial sequence
  Single strand DNA oligonucleotide
tgtcactaccttcaccttatg

SEQ ID NO: 16 - DNA, 18 bp, Artificial sequence
  Single strand DNA oligonucleotide
gctccaacccagataacg

SEQ ID NO: 17 - DNA, 27 bp, Artificial sequence
  Single strand DNA oligonucleotide
cagctacgaggagtccacctggagcac

SEQ ID NO: 18 - DNA, 27 bp, Artificial sequence
  Single strand DNA oligonucleotide
cggtagggctgcctccagaccgttcat

SEQ ID NO: 19 - DNA, 20 bp, Artificial sequence
  Single strand DNA oligonucleotide
aactccaagctggagaaggc

SEQ ID NO: 20 - DNA, 20 bp, Artificial sequence
  Single strand DNA oligonucleotide
gtcaggaactgtaccgcctc

SEQ ID NO: 21 - DNA, 20 bp, Artificial sequence
  Single strand DNA oligonucleotide
aatgactgtgaagcacctcc

SEQ ID NO: 22 - DNA, 19 bp, Artificial sequence
  Single strand DNA oligonucleotide
atttccccaacacgctcgg

SEQ ID NO: 23 - DNA, 20 bp, Artificial sequence
  Single strand DNA oligonucleotide
cctgcatcaccagagacctg

SEQ ID NO: 24 - DNA, 23 bp, Artificial sequence
  Single strand DNA oligonucleotide
ttcgacataagctcagaagggaa

SEQ ID NO: 25 - DNA, 20 bp, Artificial sequence
  Single strand DNA oligonucleotide
catcaccaacagcgaatggc

SEQ ID NO: 26 - DNA, 20 bp, Artificial sequence
  Single strand DNA oligonucleotide
cactgcttacaggaggcgaa

SEQ ID NO: 27 - DNA, 20 bp, Artificial sequence
  Single strand DNA oligonucleotide
caaccgggtcaagttggtca

SEQ ID NO: 28 - DNA, 20 bp, Artificial sequence
  Single strand DNA oligonucleotide
ctcatcttcttgttggccgc

SEQ ID NO: 29 - DNA, 19 bp, Artificial sequence
  Single strand DNA oligonucleotide
gctcgcccaggtgttttgt

SEQ ID NO: 30 - DNA, 20 bp, Artificial sequence
  Single strand DNA oligonucleotide
gcatggaacgtggttggaat

SEQ ID NO: 31 - DNA, 19 bp, Artificial sequence
  Single strand DNA oligonucleotide
agagccaggttctcctccg

SEQ ID NO: 32 - DNA, 20 bp, Artificial sequence
  Single strand DNA oligonucleotide
actagacaccaggctggcgt

SEQ ID NO: 33 - DNA, 21 bp, Artificial sequence
  Single strand DNA oligonucleotide
ggttccagaaccatcgctaca

SEQ ID NO: 34 - DNA, 20 bp, Artificial sequence
  Single strand DNA oligonucleotide
gcttcgatcctggcatccat

SEQ ID NO: 35 - DNA, 20 bp, Artificial sequence
  Single strand DNA oligonucleotide
catgaccgagagccaagtga

SEQ ID NO: 36 - DNA, 20 bp, Artificial sequence
  Single strand DNA oligonucleotide
gcttctttttagccgacgcc

SEQ ID NO: 37 - DNA, 20 bp, Artificial sequence
  Single strand DNA oligonucleotide
agcccaggtgaagacagaga

SEQ ID NO: 38 - DNA, 20 bp, Artificial sequence
  Single strand DNA oligonucleotide
agtcaaactggggtcgtgag

SEQ ID NO: 39 - DNA, 24 bp, Artificial sequence
  Single strand DNA oligonucleotide
ttgagctctcaacgaacgctttgc

SEQ ID NO: 40 - DNA, 24 bp, Artificial sequence
  Single strand DNA oligonucleotide
tcagaccctaagcaactggcagaa

SEQ ID NO: 41 - DNA, 20 bp, Artificial sequence
  Single strand DNA oligonucleotide
tggcaacttcacctaccaca

SEQ ID NO: 42 - DNA, 20 bp, Artificial sequence
  Single strand DNA oligonucleotide
gtggaacctctgcaaaaagc

SEQ ID NO: 43 - DNA, 20 bp, Artificial sequence
  Single strand DNA oligonucleotide
ggtcggtgtgaacggatttg

SEQ ID NO: 44 - DNA, 24 bp, Artificial sequence
  Single strand DNA oligonucleotide
tcgttgatggcaacaatctccact

SEQ ID NO: 45 - DNA, 60 bp, Artificial sequence
  ShRNA oligonucleotide sequence
gatctccccaacgtccagatgtactattcaagagatagtacatctggacgttggtttttc

SEQ ID NO: 46 - DNA, 60 bp, Artificial sequence
  ShRNA oligonucleotide sequence
tcgagaaaaaccaacgtccagatgtactatctcttgaatagtacatctggacgttgggga

SEQ ID NO: 47 - DNA, 60 bp, Artificial sequence
  ShRNA oligonucleotide sequence
gatctccgaaggccagtacccgtggattcaagagatccacgggtactggccttctttttc

SEQ ID NO: 48 - DNA, 59 bp, Artificial sequence
  ShRNA oligonucleotide sequence
cgagaaaaagaaggccagtacccgtggatctcttgaatccacgggtactggccttcgga

SEQ ID NO: 49 - RNA, 9 nt, Artificial sequence
  RNA oligonucleotide sequences that can be used to form a loop
uucaagaga

SEQ ID NO: 50 - RNA, 9 nt, Artificial sequence
  RNA oligonucleotide sequences that can be used to form a loop
uuuguguag

SEQ ID NO: 51 - protein, 613 aa, homo sapiens
MRAPGALLARMSRLLLLLLLKVSASSALGVAPASRNETCLGESCAPTVIQRRGRDAWGPGNSARDVLRARAPREEQGAAFLAGPSWDLPAAPGRDPAAGRGAEASAAGPPGPPTRPPGPWRWKGARGQEPSETLGRGNPTALQLFLQISEEEEKGPRGAGISGRSQEQSVKTVPGASDLFYWPRRAGKLQGSHHKPLSKTANGLAGHEGWTIALPGRALAQNGSLGEGIHEPGGPRRGNSTNRRVRLKNPFYPLTQESYGAYAVMCLSVVIFGTGIIGNLAVMCIVCHNYYMRSISNSLLANLAFWDFLIIFFCLPLVIFHELTKKWLLEDFSCKIVPYIEVASLGVTTFTLCALCIDRFRAATNVQMYYEMIENCSSTTAKLAVIWVGALLLALPEVVLRQLSKEDLGFSGRAPAERCIIKISPDLPDTIYVLALTYDSARLWWYFGCYFCLPTLFTITCSLVTARKIRKAEKACTRGNKRQIQLESQMNCTVVALTILYGFCIIPENICNIVTAYMATGVSQQTMDLLNIISQFLLFFKSCVTPVLLFCLCKPFSRAFMECCCCCCEECIQKSSTVTSDDNDNEYTTELELSPFSTIRREMSTFASVGTHC

SEQ ID NO: 52 - DNA, 3815 bp, homo sapiens
ttagtgagtggtgaaccaccaggggatcccgtctccccacaaaccagtatctctccgaggaggaggcgaaggagtgggaggaggcaacgagccgagagtcgagcttcgcgggcgcgcgcagcggctggagcgcgggggcgaggccgggccacctccccttcccggccgcgcactgcctggcccgcggcggttccaggcaccacccttcccgtccgggctgagcccgctgtggcagtgactagctcccgcggctagcggcactgtccaccgacgagcggcgccctcttctcccccttctccccacgatttccttctctgcggcggcacgccgtccagcagcctgcttcgccccgtcgtcaactttgagctggaggagaagcaactttggcagtggccgcggggttggaatcccgcttctcctcggcagcagtaggctcgcaagtcgctggggttaggtggggcaagagtttcgccggcgcatcagcgctgcttcggactgtttgcaacgtgtttccagcgagctgggagcggggttgtgactgcgagtcgtctgggggagggggacttgtttttcttttcctctagagacctcggcttgcaactggatcaaacgctgtcgaaaggatgtaaataggcagagcaactgttaccaagaaggccaccacccccacccaaaggcagtgaggagtgtggggcttcgtctgggctcccccgagtctcaacagtaatcaacagtcaggtgttgattgcaacttttcaaggtcagccaccgggagtagcctattccctctaggaaccttggagggcataccttgctgggactcaacttggctgagaaatgcacaagatgccaaaggaggaaggattatagggggcgtgtgtgtgacccccaagaccgatcttccgctatcaccctaatctccggttccccgctacccgggcgggggtgagtatgtgacatgtgcctaactctcagcagcaacttcggcagcaggtgtcgatcctaactaagcaggagctgcggctgccgggtgtgccctcaccaagccatgcgagccccgggcgcgcttctcgcccgcatgtcgcggctactgcttctgctactgctcaaggtgtctgcctcttctgccctcggggtcgcccctgcgtccagaaacgaaacttgtctgggggagagctgtgcacctacagtgatccagcgccgcggcagggacgcctggggaccgggaaattctgcaagagacgttctgcgagcccgagcacccagggaggagcagggggcagcgtttcttgcgggaccctcctgggacctgccggcggccccgggccgtgacccggctgcaggcagaggggcggaggcgtcggcagccggacccccgggacctccaaccaggccacctggcccctggaggtggaaaggtgctcggggtcaggagccttctgaaactttggggagagggaaccccacggccctccagctcttccttcagatctcagaggaggaagagaagggtcccagaggcgctggcatttccgggcgtagccaggagcagagtgtgaagacagtccccggagccagcgatcttttttactggccaaggagagccgggaaactccagggttcccaccacaagcccctgtccaagacggccaatggactggcggggcacgaagggtggacaattgcactcccgggccgggcgctggcccagaatggatccttgggtgaaggaatccatgagcctgggggtccccgccggggaaacagcacgaaccggcgtgtgagactgaagaaccccttctacccgctgacccaggagtcctatggagcctacgcggtcatgtgtctgtccgtggtgatcttcgggaccggcatcattggcaacctggcggtgatgtgcatcgtgtgccacaactactacatgcggagcatctccaactccctcttggccaacctggccttctgggactttctcatcatcttcttctgccttccgctggtcatcttccacgagctgaccaagaagtggctgctggaggacttctcctgcaagatcgtgccctatatagaggtcgcttctctgggagtcaccactttcaccttatgtgctctgtgcatagaccgcttccgtgctgccaccaacgtacagatgtactacgaaatgatcgaaaactgttcctcaacaactgccaaacttgctgttatatgggtgggagctctattgttagcacttccagaagttgttctccgccagctgagcaaggaggatttggggtttagtggccgagctccggcagaaaggtgcattattaagatctctcctgatttaccagacaccatctatgttctagccctcacctacgacagtgcgagactgtggtggtattttggctgttacttttgtttgcccacgcttttcaccatcacctgctctctagtgactgcgaggaaaatccgcaaagcagagaaagcctgtacccgagggaataaacggcagattcaactagagagtcagatgaactgtacagtagtggcactgaccattttatatggattttgcattattcctgaaaatatctgcaacattgttactgcctacatggctacaggggtttcacagcagacaatggacctccttaatatcatcagccagttccttttgttctttaagtcctgtgtcaccccagtcctccttttctgtctctgcaaacccttcagtcgggccttcatggagtgctgctgctgttgctgtgaggaatgcattcagaagtcttcaacggtgaccagtgatgacaatgacaacgagtacaccacggaactcgaactctcgcctttcagtaccatacgccgtgaaatgtccacttttgcttctgtcggaactcattgctgaaggacagtacttggttgggtcagatttatttgtttgattttcatatcccgtgaaagtttttaattcatatttttccttatagggaaaaatgcaaaaaagaaacaataaagaaagaaatattaactactgtagaactgattttacaaattaatatttgtgctttgaaaaaaagtttctatttagttatttaagaagaatgagaaggccaatagttttagattattttatctggtatggtgctaatattttatttgaaaaaagttactgcaacttaacttaaaattgctaacgttttttcttcttttaaaaatacaattattgtatattgattatagcaatgtgattttgtaggttattttatatttgagttgtgattgaaagtatgttgtatatggtattgtgagatgatttgtacttggaagcattcacaaagtagcaccaaataaattacactttattctttaatgtcattgtcaatctacttttaaccaatattcaataaatcttctaattgccttaaagatacaattactggttctatgcacaatttaaaaccggccttactgttttataacgtattttcttttaaggcaggtaatcattatgttataaagaagtttttctaatagcagtattttatatgcatgattcataaaactatgttgtatgttaaaacaaagctgtatttttaatattcaggtatagatgtcaaattacttctgaatacttataaaatatgaataaatagcagagtaggaagaaagtttcttttttaaaaaattcacctctgaactagcacatagagctacagattttcccttggggaattatgggcagaatcaagaattttaaaatgcagttgtcatctgatttcctctgaacactgacctttgaagctttgtgaatcctacgtaaagcactctg

SEQ ID NO: 53 - DNA, 1174 bp, homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgaggtgggccagtccccagcagcagtggggctgggggcaggggagcaggggggcacgtag

SEQ ID NO: 54 - DNA, 1770 bp, homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctggcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 55 - DNA, 1902 bp, homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 56 - DNA, 5916 bp, homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 57 - DNA, 1514 bp, homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggtacctggagtttaatacagtgagctctagcaagggaggcgctgccttttgtttctagaatattatgttcctcaaggtccattattttgtattcttttccaagctccttattggaaggtatttttttaaatttagaattaaaaattatttagaaagttacatataaaaaaaaaaaaaaaaaa

SEQ ID NO: 58 - DNA, 27 bp, Artificial Sequence
  Single strand DNA oligonucleotide
gggtgggattagataaatgcctgctct

SEQ ID NO: 59 - DNA, 25 bp, Artificial Sequence
  Single strand DNA oligonucleotide
ggccaagagagaattggagatgctc

SEQ ID NO: 60 - DNA, 25 bp, Artificial Sequence
  Single strand DNA oligonucleotide
aacgggtctgcagatgactgggttc

SEQ ID NO: 61 - DNA, 25 bp, Artificial Sequence
  Single strand DNA oligonucleotide
ggccaagagagaattggagatgctc